(19) United States
(12) Patent Application Publication
(10) Pub. No.: US 2015/0353626 A1
(43) Pub. Date: Dec. 10, 2015

(54) APOLIPOPROTEIN A-I DERIVED PEPTIDES FOR TREATMENT OF HYPERGLYCAEMIA

(71) Applicants: Karin STENKULA, (US); Jens LAGERSTEDT, (US)

(72) Inventors: Jens Lagerstedt, Farhult (SE); Karin Stenkula, Dalby (SE)

(21) Appl. No.: 14/760,610

(22) PCT Filed: Jan. 27, 2014
(86) PCT No.: PCT/EP2014/051502

§ 371 Date: Jul. 13, 2015

(30) Foreign Application Priority Data
Jan. 25, 2013 (SE) 1300064-1

(51) Int. Cl. C07K 14/775; A61K 47/48; A61K 35/12; A61K 45/06; A61K 38/17

(52) U.S. Cl. CPC C07K 14/775; A61K 45/06; A61K 38/1709; A61K 35/12; A61K 47/48415; A61K 47/48215; A61K 47/48038

(57) ABSTRACT

The present invention relates to peptides derived from apolipoprotein A-I (apoA-I) and their use for treatment or prevention of diseases and disorders associated with hyper-glycaemia.

### FIELD OF INVENTION

The present invention relates to peptides derived from apolipoprotein A-I (apoA-I) and their use for treatment or prevention of disorders associated with hyper-glycaemia.

### BACKGROUND OF INVENTION

Apolipoprotein A-I (apoA-I) is the primary protein component of high-density lipoprotein (HDL) and as such is important for reverse cholesterol transport (Zannis et al (2006) J. Mol. Med. 84:276-294).

ApoA-I and various fragments thereof are known in the literature. Examples include US 2011/0178029 which describes modified human apoA-I polypeptides and various uses thereof. The supporting material of Qin et al (2011) Angewandte Chemie 50:12218-12221 discloses long lists of peptides generated by trypsin cleavage of various proteins and subsequently analysed by mass spectrometry, among those a number of apoA-I peptides. CN 102323365A also discloses a fragment of apoA-I. It is also known how to produce apoA-I recombinantly as evidenced e.g. by WO94/138189 which discloses an expression system for producing apoA-I-M (Milano) in E. coli.

The apoA-I protein exists in a variety of structural organisations in the different forms of HDL and in the lipid-free state (Rye et al (2000) J. Lipid Res. 50(Suppl.):S195-S200). The lipid-bound forms include both discoidal planar HDL particles of different diameters and mature spherical HDL particles of varying sizes and lipid compositions. Key features of apoA-I that determine its function include high structural plasticity resulting in major changes in secondary, tertiary, and quaternary structures between apo- and lipid-bound states, along with an amphipathic character of the helices formed by lipid association.

Lipid-free/lipid-poor apoA-I is normally present in plasma but only accounts for up to 5% of the total plasma apoA-I and accepts the cholesterol and phospholipids that efflux from cell membranes. Progressive lipidation of apoA-I generates discoidal HDL and recycles apoA-I back into the HDL fraction. Thus, the full-length apoA-I is a hydrophobic molecule that needs to be synthetically integrated with HDL before therapeutic administration as disclosed in WO 2012/28526, US 2012/0021982 and US 2011/212139.

Biophysical exploitation of the biophysical properties of apoA-I has been suggested e.g. in WO 2012/153620 which discloses coupling of apoA-I to various therapeutic peptides with the aim of increasing solubility and binding to and transport across mucosal membranes. Another example is US 2008/0227686 which discloses full length and fragments of apoA-I for use in promoting lipid influx into cells.

In relation to indications associated with hyperglycaemia it is known that the rate of glucose uptake in skeletal muscle, the principal site for plasma glucose clearance, is determined by cell surface levels of GLUT4, which is controlled by both the insulin signalling pathway and the AMP kinase (AMPK) contraction-induced pathway (Tremblay et al (2003) Front. Biosci. 8:d1072-d1084).

It has furthermore been shown that lipid-free apoA-I and spherical HDL can induce glucose uptake in C2C12 myotubes (Han et al (2007) Diabetologia 50:1960-1968) and in human myotubes differentiated from muscle satellite cells from diabetic donors (Drew et al (2009) Circulation 119:2103-2111) via the activation of AMPK. Despite these findings, prior to the priority date of the present application it was not clear whether discoidal HDL was capable of specifically regulating muscle glucose uptake and whether this occurs via AMPK. Neither was it known which domains of apo-I that elicited said effect.

### SUMMARY OF INVENTION

Given that HDL subspecies interact differently with cellular receptors at the vascular wall for cholesterol efflux and that discoidal HDL is a potent structure for this interaction (Favari et al (2009) Biochemistry 48:11067-11074), the present inventors hypothesised that discoidal HDL would be highly effective in the stimulation of glucose uptake in muscle. The inventors thus investigated the effects of synthetic discoidal HDL (rHDL) and apoA-I-derived peptides on glucose uptake, intracellular signaling, and GLUT4 translocation to the plasma membrane (sarcolemma) using L6 myotubes and isolated primary skeletal flexor digitorum brevis (FDB) fibers. The inventors have thus surprisingly found that peptides derived from the C-terminal of apoA-I, without association with lipid bodies, are capable of inducing glucose uptake in cells and thus have beneficial effects on blood glucose levels. Hence, such peptides are useful for treatment of diseases characterised by increased glucose levels in the blood.

Thus, the present invention provides an isolated polypeptide for use in a method of treatment or prevention of diseases characterised by hyperglycaemia and/or insulin resistance, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1,
   wherein said polypeptide has a length that is less than 100 amino acids, and wherein said biological activity is induction of glucose uptake in cells.

The present inventors have identified bioactive peptides derived from the C-terminal domain of apoA-I such as an isolated polypeptide consisting of, or consisting essentially of, an amino acid sequence selected from the group consisting of the amino acid sequence of SEQ ID NO: 3, 4, 6 and 9 to 1035, as well as polynucleotides encoding said polypeptides, and vectors such as expression vectors comprising said polynucleotide, and host cells comprising said polynucleotide and/or said vector.

In certain aspect the invention also concerns a pharmaceutical composition comprising said polypeptides, said polynucleotides, said vectors and said host cells.

In one aspect the invention concerns an isolated polynucleotide for use in a method of treatment or prevention of metabolic and/or vascular diseases characterised by hyperglycaemia and/or insulin resistance, said polynucleotide comprising a nucleic acid sequence which upon expression encodes a polypeptide as defined herein above.

In another aspect the invention concerns vectors comprising said polynucleotide, and host cells comprising said polynucleotide and/or said vector, for use in a method of treatment or prevention of metabolic and/or vascular diseases characterised by hyperglycaemia and/or insulin resistance.

In certain aspects the invention concerns use of an agent selected from the group consisting of:
a) an isolated polypeptide as defined herein;
b) an isolated polynucleotide as defined herein;
c) a vector as defined as defined herein; and
d) an isolated cell as defined herein,
   for the preparation of a medicament for the treatment and/or prevention of metabolic and/or vascular diseases characterised by hyperglycaemia and/or insulin resistance.

The present inventors have found that the C-terminal domain of apoA-I and peptide fragments thereof results in improved glucose clearance in the blood. Polynucleotides encoding said polypeptides and peptides are thus useful as therapeutic agents for treatment of disorders associated with high glucose levels in blood of mammals. The present invention thus concerns use of an agent selected from the group consisting of:
a) an isolated polypeptide comprising:
   i) the amino acid sequence of SEQ ID NO: 1; or
   ii) a biologically active sequence variant of the amino acid sequence of i) wherein the variant has at least 70% sequence identity to said SEQ ID NO: 1,
   iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c),
for the preparation of a medicament for the treatment and/or prevention of metabolic and/or vascular diseases characterised by hyperglycaemia and/or insulin resistance.

The invention also concerns a method for treatment of metabolic and/or vascular diseases characterised by hyperglycaemia and/or hyperinsulinaemia, said method comprising administering to an individual in need thereof a therapeutically effective amount of an agent selected from the group consisting of:
a) an isolated polypeptide comprising:
   i) the amino acid sequence of SEQ ID NO: 1; or
   ii) a biologically active sequence variant of the amino acid sequence of i) wherein the variant has at least 70% sequence identity to said SEQ ID NO: 1,
   iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c),

Additionally, the invention in one aspect concerns a method for reducing blood glucose, the method comprising contacting a mammal with an effective amount of an agent selected from the group consisting of:
a) an isolated polypeptide comprising:
   i) the amino acid sequence of SEQ ID NO: 1; or
   ii) a biologically active sequence variant of the amino acid sequence of i) wherein the variant has at least 70% sequence identity to said SEQ ID NO: 1,
   iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c).

### DESCRIPTION OF DRAWINGS

FIG. 1. Discoidal HDL is effective at inducing glucose uptake and translocation of GLUT4 glucose transporter in muscle. a) Structure models of apoA-I in lipid-free (left structures), discoidal HDL (middle structure) and spherical HDL (right structure) states indicating significant structural rearrangements in HDL maturation. Light grey areas indicates the core of phospholipids, cholesterol and cholesteryl esters. b) Glucose uptake in L6 myotubes. After 2 hours serum starvation, L6 myotubes were stimulated with 50 μg/ml (1.6 μmol/l) apoA-I or insulin (100 nmol/l) and compared with non-stimulated control myotubes (C). c) Similarly, serum-starved L6 myotubes were stimulated with 50 μg/ml (1.6 μmol/l) rHDL (apoA-I/POPC) or insulin (100 nmol/l), or non-stimulated (C) followed by glucose uptake assay. d) changes in HA-GLUT4-GFP localization after 60 minute treatment of flexor digitorum brevis (FDB) muscle fibers with 50 μg/ml (apoA-I) rHDL, 100 nmol/l insulin or no stimulation. Myofibers were fixed and immunofluorescence performed. Size bars are 10 μm. Images are representative of 3 separate experiments each with myofibers from 2-4 animals. (***p=≦0.001 in b, n=4 and c, n=3)

FIG. 2. Role of phospholipids in rHDL stimulated glucose uptake. a) After 2 hours serum starvation, L6 myotubes were stimulated with 50 μg/ml (1.6 μmol/l; apo A-I) DMPC rHDL, insulin (100 nmol/l), or 0.16 mmol/l or 2.29 mmol/l DMPC 100 nm vesicles for 60 minutes before glucose uptake was determined (n=3) *p=≦0.05, **p=≦0.01. b) Coomassie stained blue-native PAGE showing lipid-free apoA-I (4.5 μg), POPC rHDL (4.9 μg apoA-I) and DMPC rHDL (5.6 μg apoA-I).

FIG. 3. Effect of rHDL on phosphorylation of AMPK, ACC and Akt in L6 myotubes. Myotubes were serum starved for 4 hours then treated for 60 minutes with rHDL (700 μg/ml apoA-I), 2 mmol/l or 0.4 mmol/l phenformin, 100 nmol/l or 1 nmol/l insulin, or control (C). a) densitometric analysis of lysates subjected to western blot analysis using antibodies for phospho-AMPK and AMPK, c) phospho-ACC and AMPK, and f) phospho-Akt and Akt. b), d) and e) show representative Western blots. Results are of 3 independent experiments except 1 nmol/l insulin which is n=2. Error bars show SEM except in f which are SD; *p=≦0.05, **p=≦0.01.

FIG. 4. Effect of specific regions of apoA-I on glucose uptake. a) Schematic linear presentation of apoA-I fragments analyzed for their potency in inducing glucose uptake (FL apoA-I, full length apoA-I). b) Glucose uptake was measured in L6 myotubes which had been serum starved for 2 hours then stimulated with the peptides representing residues 1-189, 44-189, 44-243 or 190-243 (SEQ ID NO: 1) of human apoA-I (all 1.6 μmol/l equimolar to 50 μg/ml full length (FL) apoA-I) or 100 nmol/l insulin (Ins), or non-stimulated (C) for 60 minutes. (n=3-4; *p=≦0.05, ***p=≦0.001). c) Immunofluorescence images of FDB fibres from the HA-GLUT4-GFP transgenic mouse treated ex vivo with 190-243 fragment of apoA-I (SEQ ID NO: 1) (1.6 μmol/l), insulin (100 nmol/l) or non-stimulated for 60 minutes. Size bars are 10 μm. Images are indicative of 3 independent experiments with FDB fibers from 2-4 mice per experiment.

FIG. 5. Properties of the 190-243 apoA-I fragment of SEQ ID NO:1. a) Native gel analysis of 190-243 fragment in solution (190-243; 0.26 μg) and after incubation with L6 myotubes for 60 minutes (190-243 media; 0.26 μg). Lipid-free apoA-I (apo A-I; 4.4 μg) and rHDL (apoA-I; 9.4 μg) are used as reference samples. The band in the rHDL/apoA-I lanes between 242 kDa and 146 kDa corresponds to rHDL, while the band between 66 kDa and 20 kDa corresponds to full length apo A-I (1-243). The bands between 66 kDa and 20 kDa in the 190-243 and 190-243 media lanes correspond to 190-243 oligomers. b) Circular dichroism spectra of full length apoA-I (solid trace) and 190-243 fragment (dotted line) analyzed in PBS buffer solution at a protein concentration of 0.2 mg/ml.

FIG. 6. Lipid-free apoA-I 190-243 fragment may adopt a structure similar to that in rHDL. Left, structure of the 190-243 fragment shown in dark grey (top) as folded in the lipid-free structure model (bottom). Right, structure of the 190-243 fragment shown in dark grey as an extended amphipathic alpha-helix (top) corresponding to the structural arrangement of this region of apoA-I in discoidal HDL (bottom). Arrows indicate a shift towards HDL-resembling amphipathic alpha-helical structure of lipid-free 190-243 apoA-I fragments at equilibrium in solution.

FIG. 7. Effects of apoA-I in HDL in glucose uptake and translocation of glucose transporter GLUT4. a) Structure models of apoA-I in lipid-free (left structures, Lagerstedt et al. (2012) Biochemica Biophysica Acta; 1821:448-455), discoidal HDL (middle structure, Lagerstedt et al. (2011) J of Biological Chemistry, 2011; 286:2966-2975) and spherical HDL (right structure, Silva et al (2008) PNAS 105: 12176-12181) states indicating significant structural rearrangements in HDL maturation. Core of phospholipids, cholesterol and cholesteryl esters are included in the discoidal and spherical HDL structures in light grey. b) Coomassie stained blue-native PAGE showing lipid-free human apoA-I (4.5 μg) and POPC rHDL (4.9 μg apoA-I). c) Discoidal HDL is effective at inducing glucose uptake in L6 myotubes. After 2 hours serum starvation, L6 myotubes were stimulated with 2 μmol/l (60 μg/ml) POPC rHDL (expressed as total protein concentration of apoA-I in rHDL), insulin (100 nmol/l) or 100 nm POPC vesicles (0.10 mmol/l), or non-stimulated (C), for 60 minutes before glucose uptake was determined (n=3) *p=≦0.05 d) Translocation of GLUT4 glucose transporter in muscle measured as changes in HA-GLUT4-GFP localization after 60 minutes treatment of flexor digitorum brevis (FDB) muscle fibers with 1 μmol/l rHDL (30 μg/ml apoA-I), 100 nmol/l insulin or no stimulation. Myofibers were fixed and immunofluorescence performed. White arrows (upper panel) indicate immunofluorescence signal of HA-GLUT4-GFP that is located in the plasma membrane (identified with anti-HA antibodies). Middle panel shows the GFP fluorescent signal. Bottom panel shows the merged images of the GFP and HA signals. Size bars are 10 μm. Images are representative of 3 separate experiments each with myofibers from 2-4 animals n=3, ±SEM; *p=≦0.05.

FIG. 8. Effect of specific regions of apoA-I on glucose uptake and intracellular signaling pathway. a) Schematic linear presentation of apoA-I fragments analyzed for their potency in inducing glucose uptake (FL apoA-I, full length apoA-I). b) Glucose uptake was measured in L6 myotubes which had been serum starved for 2 hours then stimulated with the peptides representing residues 1-243, 1-189, 44-189, 44-243 or 190-243 (SEQ ID NO: 1) of apoA-I at equimolar particle concentrations (1 μmol/l), or 100 nmol/l insulin (Ins), or non-stimulated (C) for 60 minutes. Peptide treatments were based on mono- or dimeric organization for 1-243, 1-189, 44-189 and 44-243 (1 μmol/l peptide units), and di- or tetrameric for 190-243 (2 μmol/l peptide units). (n=3-4; *p=≦0.05, ***p=≦0.001). c) Myotubes were serum starved for 4 hours then treated for 60 minutes with 2, 10 or 20 μmol/l of 190-243 peptide, 1 mmol/l phenformin (Phen), 100 nmol/l insulin (Ins), or control (C) followed by western blot analysis for phosphorylated AMPK (AMP-activated protein kinase; upper panel) or phosphorylated Akt (lower panel). Tubulin was used as loading control. Blots shown are representative of three independent experiments.

FIG. 9. Effect ex vivo of 190-243 region of apoA-I on translocation of GLUT4 glucose transporter in primary isolated muscle fibers and on glucose uptake in adipose cells. a) Immunofluorescence images of primary skeletal FDB (flexor digitorum brevis) fibers from the HA-GLUT4-GFP (hemagglutinin-GLUT4-green fluorescent protein) transgenic mouse treated ex vivo with 190-243 fragment (SEQ ID NO: 1) of apoA-I (2 μmol/l), insulin (100 nmol/l) or non-stimulated for 60 minutes. Size bars are 10 μm. Myofibers were fixed and immunofluorescence performed. White arrows (upper panel) indicate immunofluorescence signal of HA-GLUT4-GFP that is located in the plasma membrane (identified with anti-HA antibodies). Middle panel shows the GFP fluorescent signal. Bottom panel shows the merged images of the GFP and HA signals. Images are indicative of 3 independent experiments with FDB fibers from 2-4 mice per experiment. b) Isolated rat adipocytes were treated for 30 min with insulin (100 nmol/l), 190-243 peptide fragment (SEQ ID NO:1) (30 μg/ml) or 1-243 apoA-I protein (150 μg/ml) followed by glucose uptake measurement. Data is presented as fold increase over basal (Control). (n=3; *p=≦0.05).

FIG. 10. Effect of 190-243 region of apoA-I on in vivo glucose tolerance. a) Acute apoA-I 190-243 fragment treatment of insulin resistant C57Bl/6 mice improves glucose-disposal capacity in glucose tolerance test (GTTs). HFD mice were treated for 3 hours with a single injection (7 mg/kg body weight) of 190-243 fragment of apoA-I (squares) or NaCl (circles; Control). Mice received an i.p. glucose load (50 mg/mouse) 3 hours after injection of NaCl or 190-243 fragment of apoA-I followed by determination of (a) glucose and (c) insulin concentration at the indicated time points. (b) and (d) show the area under the curve (AUC) values of glucose and insulin levels, respectively, during the GTT. n=3-4; p-values as shown.

FIG. 11. Peptides (23- or 27-mers) within the 190-243 region of apoA-I and their influence on in vivo glucose tolerance capacity. Acute apoA-I peptide treatments of insulin resistant C57Bl/6 mice were followed by glucose-disposal capacity in glucose tolerance tests (GTTs). HFD mice were treated for 3 hours with a single injection (6-7 mg/kg body weight) of 23-27-mer peptides: a) 27-mer (amino acids 190-216); b) 23-mer (amino acids 204-226); c) 27-mer (amino acids 217-243). Mice received an i.p. glucose load (50 mg/mouse) 3 hours after injection of NaCl or peptides followed by determination of glucose concentrations at the indicated time points. d) shows the AUC values of glucose levels during the GTT. e) shows the AUC values (above basal glucose level) of glucose levels during the GTT. n=3-4.

FIG. 12. Peptides (18-mers) within the 190-243 region of apoA-I and their influence on in vivo glucose tolerance capacity. Acute apoA-I peptide treatments of insulin resistant C57Bl/6 mice were followed by glucose-disposal capacity in glucose tolerance tests (GTTs). HFD mice were treated for 3 hours with a single injection (7 mg/kg body weight) of 18-mer peptides: a) 18-mer (amino acids 190-207); b) 18-mer (amino acids 208-225); c) 18-mer (amino acids 226-243). Mice received an i.p. glucose load (50 mg/mouse) 3 hours after injection of NaCl or peptides followed by determination of glucose concentrations at the indicated time points. d) shows the AUC values of glucose levels during the GTT. e) shows the AUC values (above basal glucose level) of glucose levels during the GTT. n=3-4.

### DETAILED DESCRIPTION OF THE INVENTION

### I. Definitions

apoA-I: apoA-I (sometimes also referred to as apoA-1, apoAI and apoA1), as used herein, refers to polypeptides having the amino acid sequences of substantially purified apoA-I obtained from any species, particularly mammalian, including chimpanzee, bovine, ovine, porcine, murine, equine, and preferably human, from any source whether natural, synthetic, semi-synthetic, or recombinant. The term also refers to biologically active fragments of apoA-I obtained from any of these species, as well as to biologically active sequence variants of these and to proteins subject to posttranslational modifications.

Coding sequence: The term “coding sequence” as used herein is a polynucleotide sequence which is transcribed and translated into a polypeptide.

Expression vector: The term “expression vectors” as used herein refers to vectors that are capable of directing the expression of genes to which they are operatively-linked. In general, expression vectors of utility in recombinant DNA techniques are often in the form of plasmids.

Fragments: The polypeptide fragments according to the present invention, including any functional equivalents thereof, may in one embodiment comprise less than 150 amino acid residues, such as less than 140 amino acid residues, for example less than 130 amino acid residues, such as less than 120 amino acid residues, for example less than 110 amino acid residues, such as less than 100 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues, such as less than 45 amino acid residues, for example less than 40 amino acid residues, such as 35 amino acid residues, for example 30 amino acid residues, such as 25 amino acid residues, such as 20 amino acid residues, for example 15 amino acid residues, such as 10 contiguous amino acid residues of an amino acid sequence selected from the group consisting of SEQ ID NOs: 1 to 1035 or a variant thereof being at least 70% (e.g. at least 85%, 90%, 95%, 97%, 98%, or 99%) identical to said sequences. Also, the polypeptide fragments according to the present invention, including any functional equivalents thereof, may in one embodiment comprise more than 10 amino acid residues, for example more than 15 amino acid residues, such as more than 20 amino acid residues, for example more than 25 amino acid residues, for example more than 50 amino acid residues, such as more than 75 amino acid residues, for example more than 100 amino acid residues, such as more than 125 amino acid residues, for example more than 130 amino acid residues, such as more than 140 amino acid residues, for example more than 145 amino acid residues selected from the group consisting of SEQ ID NOs: 1 to 1035 or a variant thereof being at least 70% (e.g. at least 75%, 80%, 85%, 90%, 95%, 97%, 98%, or at least 99%) identical to said sequences. Examples of active fragments include one or more of the following: SEQ ID NO: 2 to 1035. The fragments may be from 10 to 150 amino acids in length, for example, 15 to 140, 20 to 130, 25 to 120, 25 to 110, 25 to 100, 25 to 90, 25 to 80, 25 to 70, 25 to 60, 25 to 54, 30 to 54, 20 to 40 or 20 to 30 consecutive amino acid residues selected from any one of SEQ ID NOs: 1 to 1035.

Hyperglycaemia: Hyperglycaemia is a condition in which an excessive amount of glucose circulates in the blood plasma. This is generally a glucose level higher than 11.1 mmol/l (200 mg/dl), but symptoms may not start to become noticeable until even higher values such as 15-20 mmol/l (˜250-300 mg/dl). A subject with a consistent range between 100 mg/dl and 126 mg/dl (American Diabetes Association guidelines) is considered hyperglycemic, while above 126 mg/dl or 7 mmol/l is generally held to have diabetes. Chronic levels exceeding 7 mmol/l (125 mg/dl) can produce organ damage. In general, the normal range for most people (fasting adults) is about 80 to 110 mg/dl or 4 to 6 mmol/l, (where 80 mg/dl is considered“optimal”.) Blood glucose levels may be determined by using various known techniques in the field, such as for example by use of commercially available blood glucose meters, continuous glucose monitors (CGM) or glucose sensing bio-implants. Chronic hyperglycemia at levels more than slightly above normal can produce a very wide variety of serious complications over a period of years, including kidney damage, neurological damage, cardiovascular damage, damage to the retina or damage to feet and legs. Diabetic neuropathy may be a result of long-term hyperglycemia. The following symptoms may be associated with acute or chronic hyperglycemia, with the first three composing the classic hyperglycemic triad: Polyphagia (pronounced or frequent hunger), polydipsia (excessive or frequent thirst), polyuria (frequent urination), blurred vision, fatigue, weight loss, poor wound healing, dry mouth, dry or itchy skin, tingling sensation in feet and heels, erectile dysfunction, recurrent infections such as for example external ear infections, cardiac arrhythmia, stupor, coma, seizures.

Increased: The term increased as used in connection with the plasma half-life is used to indicate that the relevant half-life of the apoA-I derived peptide, as determined under comparable conditions. For instance the relevant half-life may be increased by at least about 25%, such as by at least about 50%, e.g., by at least about 100%, 150%, 200%, 250%, or 500%. Measurement of in vivo plasma half-life can be carried out in a number of ways as described in the literature. An increase in in-vivo plasma half-life may be quantified as a decrease in clearance or as an increase in mean residence time (MRT). The apoA-I derived peptide of the present invention for which the clearance is decreased to less than 70%, such as less than 50%, such as less than 20%, such as less than 10% of the clearance of the apoA-I derived peptide, as determined in a suitable assay is said to have an increased in-vivo plasma half-life. The apoA-I derived peptide of the present invention for which MRT is increased to more than 130%, such as more than 150%, such as more than 200%, such as more than 500% of the MRT of apoA-I, in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable test animals include normal, Sprague-Dawley male rats, mice and cynomolgus monkeys. Typically the mice and rats are injected in a single subcutaneous bolus, while monkeys may be injected in a single subcutaneous bolus or in a single iv dose. The amount injected depends on the test animal. Subsequently, blood samples are taken over a period of one to ten days as appropriate (depending on the sensitivity of the assay it may be as long as 30 days) for the assessment of clearance and MRT. The blood samples are conveniently analysed by ELISA techniques or other immunological techniques.

Insulin resistance: Individuals are said to be ‘insulin resistant’, when their tissues behave as if there was insufficient insulin in the bloodstream as reflected by decreased insulin response and glucose uptake in liver, adipose tissue, and skeletal muscle. The first response to insulin resistance is a compensatory production and secretion of insulin to compensate for the body's decreased sensitivity, leading to hyperinsulinaemia. Thus, high insulin levels and a decreased responsiveness of tissue to the clearance of glucose from the bloodstream characterize insulin resistance. Insulin resistance is the primary event leading to a series of metabolic changes including compensatory hyperinsulinemia, dyslipidemia, decompensation of pancreatic beta-cells, and hyperglycemia. Signs and symptoms of insulin resistance are: Brain fogginess and inability to focus, high blood glucose levels, intestinal bloating, sleepiness, weight gain, fat storage (particularly in and around abdominal organs), difficulty losing weight, increased blood triglyceride levels, increased blood pressure, increased pro-inflammatory cytokines associated with cardiovascular disease, depression, acanthosis nigricans, increased hunger. Insulin resistance can be diagnosed by use of the different techniques in the field, for example the hyperinsulinemic euglycemic clamp, or the modified insulin suppression test which are commonly used in the field.

Linker: The term “linker” as used herein means a valence bond or multifunctional moiety, such as a bifunctional moiety that separates the apoA-I fragment and the pharmaceutically acceptable molecule conjugated to apoA-I thus resulting in increased half-life such as increased plasma half-life.

Normal lipid levels: The term ‘normal lipid levels’ as used herein is to be interpreted as blood or plasma lipid levels in accordance with recommendations from a central health authority in the jurisdiction in question. E.g. in Europe, the guidelines according to European Atherosclerosis Society (Europe): total-cholesterol <5.2 mM, LDL-cholesterol <1.8 mM, HDL-cholesterol >1.0 mM, Triglycerides <1.7 mM. Guidelines according to American Heart Association (U.S.): total-cholesterol <200 mg/dl, LDL-cholesterol <100 mg/dl, HDL-cholesterol >60 mg/dl, Triglycerides <100 mg/dl)). The lipid profile is obtained by blood sample after 12-hour fasting.

Operably linked: As used herein, the terms operably linked or operatively-linked are intended to mean that the nucleotide sequence of interest is linked to the regulatory sequence(s) within a recombinant expression vector, in a manner that allows for expression of the nucleotide sequence (e.g., in an in vitro transcription/translation system or in a host cell when the vector is introduced into the host cell).

Pharmaceutically acceptable molecule: The term a pharmaceutically acceptable molecule as used herein means a molecule selected from any one of small organic molecules, peptides, oligopeptides, polypeptides, proteins, receptors, glycosylations, sugars, polymers (e.g. polyethylene glycols, PEG), nucleic acids (e.g. DNA and RNA), hormones, which when linked to apoA-I, increases the serum half-life of the apoA-I or variant thereof. Typically, pharmaceutically acceptable molecules are without limitation albumin, such as human albumin, recombinant albumin, or polymer, such as PEG, e.g. PEG of a molecular weight of at least 10 kDa, such as from 10 kDa to 150 kDa. Furthermore, pharmaceutically acceptable molecules may be selected from a Fc fragment of a mammalian antibody, transferrin, albumin, such as human albumin, recombinant albumin, variants of albumin, CH₃(CH₂)ₙCO—, wherein n is 8 to 22, or polymer, such as PEG, e.g. PEG of a molecular weight of at least 5 kDa, such as from 10 kDa to 150 kDa, typically 10 to 40 kDa.

Plasma concentration: The term plasma concentration as used herein means the concentration that can be measured in circulation at any given time after administration of the apoA-I derived peptide of the present invention.

Polymer: The term “polymer” as used herein means a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules attached by in vitro glycosylation. Carbohydrate molecules attached by in vivo glycosylation, such as N- or O-glycosylation (as further described below) are referred to herein as “an oligosaccharide moiety”. Except where the number of polymer molecules is expressly indicated, every reference to “a polymer”, “a polymer molecule”, “the polymer” or “the polymer molecule” as used in the present invention shall be a reference to one or more polymer molecule(s). The polymer may be a water soluble or water insoluble polymer, such as a PEG moiety. The PEG moiety may have an average size selected from the range of 500 Da to 200,000 Da, such as from 500 Da to 100,000 Da, such as from 2000 Da to 50,000 Da. Such PEG molecules may be retrieved from i.e. Shearwater Inc.

Regulatory sequence: The term “regulatory sequence” as used herein is intended to include promoters, enhancers and other expression control elements (e.g., polyadenylation signals).

Serum half-life: The term serum half-life, which may be used interchangeably with “plasma half-life” or “half-life” is used in its normal meaning, i.e., the time required for the amount of the apoA-I derived peptide in a biological system to be reduced to one half of its concentration. Thus as used herein, the “serum half-life” means the serum half-life in vivo. Determination of serum half-life is often more simple than determining functional half-life and the magnitude of serum half-life is usually a good indication of the magnitude of functional in vivo half-life. Preferably the serum half-life is measured in a mammal, more preferably in a species of Hominidae, such as Orangutan, Chimpanzee or Gorillas, more preferably in humans. The serum half-lives mentioned in the present application are half-lives as determined in humans. An indication of the half-life or any change in half-life can also be obtained in rodents, such as mouse or rat or hamster. Furthermore half-life can be measured in larger mammals having a body weight in the same range as human beings or closer to human being body weight than rodents: preferably monkey, dog, pig, or cattle (calf).

SEQ ID NO. X to Z: The term ‘SEQ ID NO X to Z’ as used herein, wherein X and Z are two integers and wherein Z>X, is to be interpreted as including an interval X to Z (X-Z) including the integers X and Z and each and every integer in between X and Z, such as X, Y, Z. Thus the term SEQ ID NOs: 1 to 8 is to be interpreted as an interval including SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8. Similarly, SEQ ID NO: 1 to 1035 and SEQ ID NO: 1-1035 is to be interpreted as including the sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 272, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 407, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 454, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 503, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, SEQ ID NO: 550, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, SEQ ID NO: 588, SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, SEQ ID NO: 630, SEQ ID NO: 631, SEQ ID NO: 632, SEQ ID NO: 633, SEQ ID NO: 634, SEQ ID NO: 635, SEQ ID NO: 636, SEQ ID NO: 637, SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 700, SEQ ID NO: 701, SEQ ID NO: 702, SEQ ID NO: 703, SEQ ID NO: 704, SEQ ID NO: 705, SEQ ID NO: 706, SEQ ID NO: 707, SEQ ID NO: 708, SEQ ID NO: 709, SEQ ID NO: 710, SEQ ID NO: 711, SEQ ID NO: 712, SEQ ID NO: 713, SEQ ID NO: 714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, SEQ ID NO: 731, SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 770, SEQ ID NO: 771, SEQ ID NO: 772, SEQ ID NO: 773, SEQ ID NO: 774, SEQ ID NO: 775, SEQ ID NO: 776, SEQ ID NO: 777, SEQ ID NO: 778, SEQ ID NO: 779, SEQ ID NO: 780, SEQ ID NO: 781, SEQ ID NO: 782, SEQ ID NO: 783, SEQ ID NO: 784, SEQ ID NO: 785, SEQ ID NO: 786, SEQ ID NO: 787, SEQ ID NO: 788, SEQ ID NO: 789, SEQ ID NO: 790, SEQ ID NO: 791, SEQ ID NO: 792, SEQ ID NO: 793, SEQ ID NO: 794, SEQ ID NO: 795, SEQ ID NO: 796, SEQ ID NO: 797, SEQ ID NO: 798, SEQ ID NO: 799, SEQ ID NO: 800, SEQ ID NO: 801, SEQ ID NO: 802, SEQ ID NO: 803, SEQ ID NO: 804, SEQ ID NO: 805, SEQ ID NO: 806, SEQ ID NO: 807, SEQ ID NO: 808, SEQ ID NO: 809, SEQ ID NO: 810, SEQ ID NO: 811, SEQ ID NO: 812, SEQ ID NO: 813, SEQ ID NO: 814, SEQ ID NO: 815, SEQ ID NO: 816, SEQ ID NO: 817, SEQ ID NO: 818, SEQ ID NO: 819, SEQ ID NO: 820, SEQ ID NO: 821, SEQ ID NO: 822, SEQ ID NO: 823, SEQ ID NO: 824, SEQ ID NO: 825, SEQ ID NO: 826, SEQ ID NO: 827, SEQ ID NO: 828, SEQ ID NO: 829, SEQ ID NO: 830, SEQ ID NO: 831, SEQ ID NO: 832, SEQ ID NO: 833, SEQ ID NO: 834, SEQ ID NO: 835, SEQ ID NO: 836, SEQ ID NO: 837, SEQ ID NO: 838, SEQ ID NO: 839, SEQ ID NO: 840, SEQ ID NO: 841, SEQ ID NO: 842, SEQ ID NO: 843, SEQ ID NO: 844, SEQ ID NO: 845, SEQ ID NO: 846, SEQ ID NO: 847, SEQ ID NO: 848, SEQ ID NO: 849, SEQ ID NO: 850, SEQ ID NO: 851, SEQ ID NO: 852, SEQ ID NO: 853, SEQ ID NO: 854, SEQ ID NO: 855, SEQ ID NO: 856, SEQ ID NO: 857, SEQ ID NO: 858, SEQ ID NO: 859, SEQ ID NO: 860, SEQ ID NO: 861, SEQ ID NO: 862, SEQ ID NO: 863, SEQ ID NO: 864, SEQ ID NO: 865, SEQ ID NO: 866, SEQ ID NO: 867, SEQ ID NO: 868, SEQ ID NO: 869, SEQ ID NO: 870, SEQ ID NO: 871, SEQ ID NO: 872, SEQ ID NO: 873, SEQ ID NO: 874, SEQ ID NO: 875, SEQ ID NO: 876, SEQ ID NO: 877, SEQ ID NO: 878, SEQ ID NO: 879, SEQ ID NO: 880, SEQ ID NO: 881, SEQ ID NO: 882, SEQ ID NO: 883, SEQ ID NO: 884, SEQ ID NO: 885, SEQ ID NO: 886, SEQ ID NO: 887, SEQ ID NO: 888, SEQ ID NO: 889, SEQ ID NO: 890, SEQ ID NO: 891, SEQ ID NO: 892, SEQ ID NO: 893, SEQ ID NO: 894, SEQ ID NO: 895, SEQ ID NO: 896, SEQ ID NO: 897, SEQ ID NO: 898, SEQ ID NO: 899, SEQ ID NO: 900, SEQ ID NO: 901, SEQ ID NO: 902, SEQ ID NO: 903, SEQ ID NO: 904, SEQ ID NO: 905, SEQ ID NO: 906, SEQ ID NO: 907, SEQ ID NO: 908, SEQ ID NO: 909, SEQ ID NO: 910, SEQ ID NO: 911, SEQ ID NO: 912, SEQ ID NO: 913, SEQ ID NO: 914, SEQ ID NO: 915, SEQ ID NO: 916, SEQ ID NO: 917, SEQ ID NO: 918, SEQ ID NO: 919, SEQ ID NO: 920, SEQ ID NO: 921, SEQ ID NO: 922, SEQ ID NO: 923, SEQ ID NO: 924, SEQ ID NO: 925, SEQ ID NO: 926, SEQ ID NO: 927, SEQ ID NO: 928, SEQ ID NO: 929, SEQ ID NO: 930, SEQ ID NO: 931, SEQ ID NO: 932, SEQ ID NO: 933, SEQ ID NO: 934, SEQ ID NO: 935, SEQ ID NO: 936, SEQ ID NO: 937, SEQ ID NO: 938, SEQ ID NO: 939, SEQ ID NO: 940, SEQ ID NO: 941, SEQ ID NO: 942, SEQ ID NO: 943, SEQ ID NO: 944, SEQ ID NO: 945, SEQ ID NO: 946, SEQ ID NO: 947, SEQ ID NO: 948, SEQ ID NO: 949, SEQ ID NO: 950, SEQ ID NO: 951, SEQ ID NO: 952, SEQ ID NO: 953, SEQ ID NO: 954, SEQ ID NO: 955, SEQ ID NO: 956, SEQ ID NO: 957, SEQ ID NO: 958, SEQ ID NO: 959, SEQ ID NO: 960, SEQ ID NO: 961, SEQ ID NO: 962, SEQ ID NO: 963, SEQ ID NO: 964, SEQ ID NO: 965, SEQ ID NO: 966, SEQ ID NO: 967, SEQ ID NO: 968, SEQ ID NO: 969, SEQ ID NO: 970, SEQ ID NO: 971, SEQ ID NO: 972, SEQ ID NO: 973, SEQ ID NO: 974, SEQ ID NO: 975, SEQ ID NO: 976, SEQ ID NO: 977, SEQ ID NO: 978, SEQ ID NO: 979, SEQ ID NO: 980, SEQ ID NO: 981, SEQ ID NO: 982, SEQ ID NO: 983, SEQ ID NO: 984, SEQ ID NO: 985, SEQ ID NO: 986, SEQ ID NO: 987, SEQ ID NO: 988, SEQ ID NO: 989, SEQ ID NO: 990, SEQ ID NO: 991, SEQ ID NO: 992, SEQ ID NO: 993, SEQ ID NO: 994, SEQ ID NO: 995, SEQ ID NO: 996, SEQ ID NO: 997, SEQ ID NO: 998, SEQ ID NO: 999, SEQ ID NO: 1000, SEQ ID NO: 1001, SEQ ID NO: 1002, SEQ ID NO: 1003, SEQ ID NO: 1004, SEQ ID NO: 1005, SEQ ID NO: 1006, SEQ ID NO: 1007, SEQ ID NO: 1008, SEQ ID NO: 1009, SEQ ID NO: 1010, SEQ ID NO: 1011, SEQ ID NO: 1012, SEQ ID NO: 1013, SEQ ID NO: 1014, SEQ ID NO: 1015, SEQ ID NO: 1016, SEQ ID NO: 1017, SEQ ID NO: 1018, SEQ ID NO: 1019, SEQ ID NO: 1020, SEQ ID NO: 1021, SEQ ID NO: 1022, SEQ ID NO: 1023, SEQ ID NO: 1024, SEQ ID NO: 1025, SEQ ID NO: 1026, SEQ ID NO: 1027, SEQ ID NO: 1028, SEQ ID NO: 1029, SEQ ID NO: 1030, SEQ ID NO: 1031, SEQ ID NO: 1032, SEQ ID NO: 1033, SEQ ID NO: 1034, and SEQ ID NO: 1035.

Sequence identity: A high level of sequence identity indicates likelihood that the first sequence is derived from the second sequence. Amino acid sequence identity requires identical amino acid sequences between two aligned sequences. Thus, a candidate sequence sharing 70% amino acid identity with a reference sequence, requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. The ClustalW software is available as a ClustalW WWW Service at the European Bioinformatics Institute from http://www.ebi.ac.uk/clustalw. Using this program with its default settings, the mature (bioactive) part of a query and a reference polypeptide are aligned. The number of fully conserved residues are counted and divided by the length of the reference polypeptide.

The ClustalW algorithm may similarly be used to align nucleotide sequences. Sequence identities may be calculated in a similar way as indicated for amino acid sequences.

Subject: The term “subject” used herein is taken to mean any mammal to which the apoA-I polypeptide fragment or polynucleotide or therapeutic cells or biocompatible capsules may be administered. Subjects specifically intended for treatment with the method of the invention include humans, as well as nonhuman primates, sheep, horses, cattle, goats, pigs, dogs, cats, rabbits, guinea pigs, hamsters, gerbils, rats and mice, as well as the organs, tumours, and cells derived or originating from these hosts.

Transformation: The term transformation as used herein refers to the insertion of an exogenous polynucleotide (i.e., a “transgene”) into a host cell. The exogenous polynucleotide is integrated within the host genome.

Pharmaceutical agent: The terms “pharmaceutical agent” or “drug” or “medicament” refer to any therapeutic or prophylactic use of an agent according to the invention, which agent may be used in the treatment (including the prevention, diagnosis, alleviation, or cure) of a malady, affliction, condition, disease or injury in a patient. Therapeutically useful genetic determinants, peptides, polypeptides and polynucleotides may be included within the meaning of the term pharmaceutical or drug. As defined herein, a “therapeutic agent”, “pharmaceutical agent” or “drug” or “medicament” is a type of bioactive agent.

Pharmaceutical composition: or drug, medicament or agent refers to any chemical or biological material, compound, or composition capable of inducing a desired therapeutic effect when properly administered to a patient. Some drugs are sold in an inactive form that is converted in vivo into a metabolite with pharmaceutical activity. For purposes of the present invention, the terms “pharmaceutical composition” and “medicament” preferably encompass an active agent as such or an inactive drug and the active metabolite.

Variants: The term “variants” as used herein refers to amino acid sequence variants said variants preferably having at least 60% identity, for example at least 63% identity, such as at least 66% identity, for example at least 70% sequence identity, for example at least 72% sequence identity, for example at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with any of the predetermined sequences of SEQ ID NOs: 1 to 1035.

Vector: The term “vector” as used herein refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### II. Indications and Treatment

### Metabolic Diseases

The present inventors have surprisingly discovered that the C-terminal amino acid sequence of apoA-I is a physically stable molecule in hydrophilic solutes, while the glucose uptake properties persist. For a person working in the field it is expected that the physical stability will be kept also when the presently active peptide is further truncated to a shorter peptide that includes the medicinal effect.

Thus the present inventors have found that peptides derived from the C-terminal of apoA-I, are capable of inducing glucose uptake in cells and thus have beneficial effects on blood glucose levels. Hence, such peptides have an effect on plasma glucose clearance and are useful for treatment of diseases characterised by increased glucose levels in the blood.

The therapeutic effect of this finding is that the peptides are useful for the treatment of metabolic disease, or for the prophylactic treatment of a mammal facing the risk of developing metabolic disease.

In one aspect the present invention concerns an isolated polypeptide for use in a method of treatment or prevention of diseases characterised by hyperglycaemia and/or insulin resistance, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1
   wherein said polypeptide has a length that is less than 100 amino acids, and wherein said biological activity is induction of glucose uptake in cells.

In another aspect the present invention concerns an isolated polypeptide for use in a method of treatment or prevention of diseases characterised by hyperglycaemia and/or insulin resistance, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1
   wherein said biological activity is induction of glucose uptake in cells, and
   wherein said polypeptide has a length that is less than 200 amino acids, preferably less than less than 190 amino acids, more preferably less than 180 amino acids, more preferably less than 170 amino acids, more preferably less than 160 amino acids, more preferably less than 150 amino acids, more preferably less than 140 amino acids, more preferably less than 130 amino acids, more preferably less than 120 amino acids, more preferably less than 110 amino acids, more preferably less than 100 amino acids, more preferably less than 90 amino acids, more preferably less than 80 amino acids, more preferably less than 70 amino acids, more preferably less than 75 amino acids, more preferably less than 70 amino acids, more preferably less than 65 amino acids, more preferably less than 60 amino acids, more preferably less than 55 amino acids.

In one aspect the invention concerns use of an agent selected from the group consisting of:
a) an isolated polypeptide consisting of less than 100 amino acid residues and comprising:
   i) the amino acid sequence of SEQ ID NO: 1; or
   ii) a biologically active sequence variant of the amino acid sequence of i) wherein the variant has at least 70% sequence identity to said SEQ ID NO: 1,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
   for the preparation of a medicament for the treatment and/or prevention of diseases characterised by hyperglycaemia and/or insulin resistance.

In one aspect the invention concerns a method of treatment of diseases characterised by hyperglycaemia and/or insulin resistance, said method comprising administering to an individual in need thereof a therapeutically effective amount of an agent selected from the group consisting of:
a) an isolated polypeptide consisting of less than 100 amino acid residues and comprising:
   i) the amino acid sequence of SEQ ID NO: 1; or
   ii) a biologically active sequence variant of the amino acid sequence of i) wherein the variant has at least 70% sequence identity to said SEQ ID NO: 1,
   iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one aspect the invention concerns an isolated polypeptide for use in a method of treatment or prevention of diseases characterised by hyperglycaemia and/or insulin resistance, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1
   wherein said polypeptide has a length that is less than 100 amino acids.

The sequence variant can be any biologically active variant wherein the biological activity is induction of glucose uptake in cells. Preferably the variant is a sequence variant of SEQ ID NO: 1 wherein any amino acid residue of SEQ ID NO: 1 has been altered to another amino acid residue, provided that no more than 15 amino acids have been so altered, such as wherein no more than 14 amino acids have been so altered, e.g. wherein no more than 13 amino acids have been so altered, such as wherein no more than 12 amino acids have been so altered, e.g. wherein no more than 11 amino acids have been so altered, such as wherein no more than 10 amino acids have been so altered, e.g. wherein no more than 9 amino acids have been so altered, such as wherein no more than 8 amino acids have been so altered, e.g. wherein no more than 7 amino acids have been so altered, such as wherein no more than 6 amino acids have been so altered, e.g. wherein no more than 5 amino acids have been so altered, such as wherein no more than 4 amino acids have been so altered, e.g. wherein no more than 3 amino acids have been so altered, such as wherein no more than 2 amino acids have been so altered, e.g. wherein no more than 1 amino acid has been so altered in relation to said SEQ ID NO: 1.

In one embodiment the polypeptide of the present invention has at least 60% sequence identity to the amino acid sequence of SEQ ID NO: 1, more preferably at least 70% sequence identity to the amino acid sequence of SEQ ID NO: 1, more preferably at least 75% sequence identity to the amino acid sequence of SEQ ID NO: 1, more preferably at least 80% sequence identity to the amino acid sequence of SEQ ID NO: 1, more preferably at least 85% sequence identity to the amino acid sequence of SEQ ID NO: 1, more preferably 90% sequence identity to the amino acid sequence of SEQ ID NO: 1, more preferably at least 95% sequence identity to the amino acid sequence of SEQ ID NO: 1, more preferably at least 98% sequence identity to the amino acid sequence of SEQ ID NO: 1, more preferably at least 99% sequence identity to the amino acid sequence of SEQ ID NO: 1.

In another embodiment, the polypeptide of the present invention has at least 60% sequence identity to any one of the amino acid sequences of SEQ ID NO: 1 to 1035, more preferably at least 70% sequence identity to any one of the amino acid sequences of SEQ ID NO: 1 to 1035, more preferably at least 75% sequence identity to any one of the amino acid sequences of SEQ ID NO: 1 to 1035, more preferably at least 80% sequence identity to any one of the amino acid sequences of SEQ ID NO: 1 to 1035, more preferably at least 85% sequence identity to any one of the amino acid sequences of SEQ ID NO: 1 to 1035, more preferably 90% sequence identity to the amino acid sequence of to any one of the amino acid sequences of SEQ ID NO: 1 to 1035, more preferably at least 95% sequence identity to the amino acid sequence of to any one of the amino acid sequences of SEQ ID NO: 1 to 1035, more preferably at least 98% sequence identity to the amino acid sequence of to any one of the amino acid sequences of SEQ ID NO: 1 to 1035, more preferably at least 99% sequence identity to the amino acid sequence of to any one of the amino acid sequences of SEQ ID NO: 1 to 1035.

In one embodiment the polypeptide for use as defined herein consists essentially of any one of the amino acid sequences defined herein, such as consisting essentially of any one of SEQ ID NOs: 1 to 1035.

In one embodiment the polypeptide for use as defined herein consists of any one of the amino acid sequences defined herein, such as consisting of any one of SEQ ID NOs: 1 to 1035.

In one embodiment the polypeptide according to the present invention is a variant polypeptide, wherein in said variant any amino acid has been altered to provide a conservative substitution relative to the amino acid sequence of SEQ ID NO: 1.

In one embodiment the polypeptide according to the present invention comprises conserved amino acid residues at positions Ala 1, Glu 2, Tyr 3, His 4, Ala 5, Lys 6, Ala 7, Glu 9, Leu 11, Leu 14, Glu 16, Lys 17, Pro 20, Leu 22, Glu 23, Asp 24, Leu 25, Arg 26, Leu 29, Pro 31, Glu 34, Lys 37, Glu 45, Glu 46, Lys 49, Lys 50, Leu 51, Gln 54 relative to the amino acid sequence of SEQ ID NO:1.

In one embodiment the polypeptide according to the present invention comprises conserved amino acid residues at positions Glu 2, Tyr 3, Leu 11, Leu 14, Glu 16, Lys 17, Pro 20, Asp 24, Leu 29, Pro 31, Glu 34, Lys 37, Glu 45 relative to the amino acid sequence of SEQ ID NO:1.

In one embodiment, the polypeptide according to the present invention is not full length pre-pro-apoA-I.

In another embodiment, the polypeptide according to the present invention is not full length pro-apoA-I.

In another embodiment, the polypeptide according to the present invention is not full length mature apoA-I.

It is to be understood that the polypeptide exhibiting the biological activity of the present invention is a peptide derived from the C-terminal domain (i.e. AA190-243) of human apoA-I, such as SEQ ID NO: 1 or a fragment or variant thereof as defined herein.

In one embodiment, the polypeptide according to the present invention is not part of a fusion protein, i.e. in said embodiment, the polypeptide of the present invention is not linked by a peptide bond or via a peptide linker to a further therapeutically active polypeptide such as insulin or GLP-1.

The peptide of the invention is useful for treating any disease directly or indirectly associated with high glucose levels in the blood. The person of skill in the art is aware of which diseases that treatable or preventable by lowering the blood and/or plasma glucose levels. In one embodiment the disease characterised by hyperglycaemia is a metabolic disease.

Non-insulin-dependent diabetes mellitus (including adult-onset, maturity-onset, nonketotic, stable, type II and non-insulin-dependent diabetes of the young) is characterised by elevated blood glucose levels and insulin resistance in the target tissues of insulin (liver-, -muscle- and adipose tissue). Non-insulin-dependent diabetes mellitus can be treated by administering the apoA-I peptide of the present invention since the peptide improves blood glucose clearance, and potentially improve insulin sensitivity in the target tissues of insulin, which inhibit the aetiology of non-insulin-dependent diabetes mellitus (Turner et al (1999) JAMA 281(21):2005-2012; Nathan et al (2002) N Engl J Med 347:1342-1349; Kadoglou et al (2007) 30(9): 2242-2244).

Thus in one embodiment the disease treatable by the present invention is selected from the group consisting of endocrine and metabolic diseases and obesity. This group of diseases includes among others non-insulin-dependent diabetes mellitus such as adult-onset, maturity-onset, nonketotic, stable, type II and non-insulin-dependent diabetes of the young.

In one embodiment the polypeptide according to the invention is for use in the treatment of a disease characterised by hyperglycaemia and/or insulin resistance wherein said disease is abnormal glucose tolerance test including chemical and latent diabetes; impaired glucose tolerance; and/or prediabetes.

Thus in one embodiment the disease treatable by the present invention is type II diabetes and/or insulin resistance.

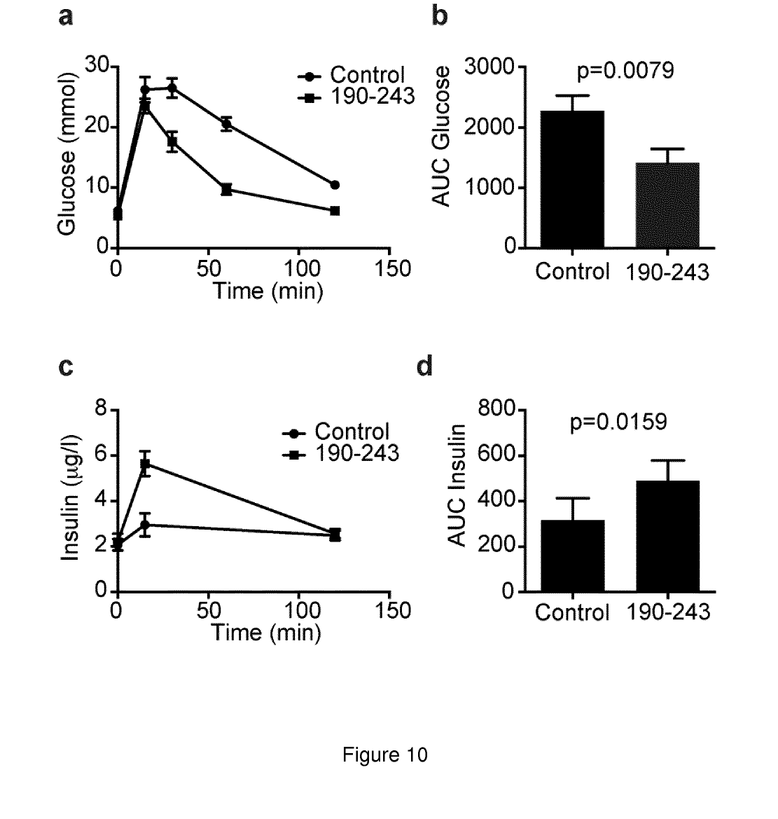

As demonstrated herein (e.g. FIG. 10) the present invention results in increased glucose clearance. Thus the invention is useful as a supplement or replacement for conventional treatment of diabetes. In one embodiment the disease treatable by the present invention is type I diabetes.

Obesity including obesity due to excess intake of calories is defined as abnormal adipose tissue accumulation to the extent that it negatively influences health, and is a major risk factor for a number of chronic diseases, including diabetes and cardiovascular diseases. The condition is most common due to increased caloric intake and lack of exercise. Obesity can be treated by administering the apoA-I peptide of the present invention because the peptide results in improved insulin sensitivity and also could result in weight loss which inhibits the aetiology of obesity by reducing risk of metabolic syndrome and diabetes associated with early mortality (Golay et al (2007) Physiol Rev. 87(2):507-20).

In one embodiment the disease treatable by the present invention is obesity e.g. obesity due to excess intake of calories such as excess intake of food.

Polycystic ovarian syndrome (including Sclerocystic ovary syndrome and Stein-Leventhal syndrome) abbreviated PCOS, is one of the most common endocrine disorders in women. Causes are assumed to be genetic but common symptoms are a number of small cysts in the ovaries, high levels of masculine hormones and irregular menstruation. The symptom of PCOS could be improved by administering the apoA-I peptide of the present invention since the peptide improve glucose clearance and thereby insulin sensitivity and could potentially improve hyper-androgenemia, reverse menstrual abnormalities and chronic anovulation associated with PCOS (Moghetti et al. (2000) J Clin Endocrinol Metab. 85(1):139-46)

Thus in one embodiment the disease treatable by the present invention is an endocrine and metabolic disease selected from the group consisting of polycystic ovarian syndrome such as Sclerocystic ovary syndrome and Stein-Leventhal syndrome.

In one embodiment the disease treatable by the present invention is a metabolic disease selected from the group consisting of: metabolic syndrome, insulin resistance, glucose intolerance, hyperglycemia, type I diabetes, type II diabetes, hyperlipidemia, hypertriglyceridemia, hypercholesterolemia, dyslipidemia, and polycystic ovary syndrome.

In one embodiment the disease treatable by the present invention is a metabolic diseases caused by insulin resistance wherein said disease is selected from the group of insulin resistance in the liver, insulin resistance in the skeletal muscles and/or insulin resistance in adipose tissue.

In one embodiment the peptides of the present invention is for use in the treatment and/or prophylaxis of metabolic syndrome, insulin resistance, glucose intolerance, hyperglycemia, type I diabetes, type II diabetes, hyperlipidemia, hypertriglyceridemia, hypercholesterolemia, dyslipidemia, and polycystic ovary syndrome. The therapeutic effect of the polypeptides of the invention is relevant for the treatment of diseases related insulin resistance in the skeletal muscles, but also for insulin resistance in the liver, or insulin resistance in adipose tissue.

In addition to the peptide derived from the C-terminal domain of apoA-I, the present invention also concerns medical use of the polynucleotides encoding any one of the biologically active polypeptides, variants and fragments of SEQ ID NO: 1.

Thus in one aspect the present invention concerns an isolated polynucleotide for use in a method of treatment or prevention of diseases characterised by hyperglycaemia and/or insulin resistance, said polynucleotide comprising a nucleic acid sequence which upon expression encodes a polypeptide as defined herein.

In a further aspect the present invention also concerns a vector for use in a method of treatment or prevention of diseases characterised by hyperglycaemia and/or insulin resistance, said vector comprising a polynucleotide comprising a nucleic acid sequence which upon expression encodes a polypeptide as defined herein. In one embodiment the vector further comprises a promoter operably linked to the polynucleotide.

The vector may be any vector suitable for expressing the polypeptides of the invention, and the vector may thus be elected by the person of skill in the art. In one embodiment the vector is selected form the group consisting of alphavirus, adenovirus, adeno associated virus, baculovirus, HSV, coronavirus, Bovine papilloma virus, and Mo-MLV, preferably adeno associated virus.

In a further aspect the present invention also concerns an isolated host cell for use in a method of treatment or prevention of diseases characterised by hyperglycaemia and/or insulin resistance, wherein said cell is transformed or transduced with the polynucleotide and/or the vector as defined herein. The cell may be any host cell suitable for accommodating the polynucleotides or vector of the invention. Preferably the cell is a human cell.

In one embodiment the cell is selected from the group consisting of stem cells, muscle cells, hepatocytes, adipocytes and cells of the pancreas such as α cells, β cells and δ cells.

In another embodiment the cell is selected from the group consisting of CHO, CHO-K1, HEI193T, HEK293, COS, HiB5, RN33b and BHK cells.

In one aspect the invention concerns use of an agent selected from the group consisting of:
a) an isolated polypeptide as defined herein;
b) an isolated polynucleotide as defined herein;
c) a vector as defined herein; and/or
d) an isolated cell as defined herein,
   for the preparation of a medicament for the treatment and/or prevention of diseases characterised by hyperglycaemia and/or insulin resistance.

In one embodiment the polypeptide according to the present invention is for use in the treatment of diseases characterised by hyperglycaemia and/or insulin resistance.

In a further embodiment, the polypeptide according to the present invention is also for use in the treatment of diseases characterised by hyperinsulinaemia.

### Cardiovascular Diseases

Other conditions being directly or indirectly associated with hyperglycaemia are various cardiovascular disorders. By lowering the concentrations of circulating blood glucose, pathological mechanisms of the heart and blood vessels can be prevented and/or inhibited.

Hence, in a further aspect the present invention concerns an isolated polypeptide for use in a method of treatment or prevention of cardiovascular diseases resulting from hyperglycaemia, said polypeptide comprising an amino acid sequence selected from the group consisting of:
a) the amino acid sequence of SEQ ID NO: 1; and
b) a biologically active sequence variant of a), wherein the variant has at least 70% sequence identity to SEQ ID NO:1,
   wherein said polypeptide has a length that is less than 100 amino acids, and wherein said biological activity is induction of glucose uptake in cells.

In another aspect the present invention concerns an isolated polypeptide for use in a method of treatment or prevention of cardiovascular diseases, said polypeptide comprising an amino acid sequence selected from the group consisting of:
a) the amino acid sequence of SEQ ID NO: 1; and
b) a biologically active sequence variant of a), wherein the variant has at least 70% sequence identity to SEQ ID NO:1,
   wherein said biological activity is induction of glucose uptake in cells, and
   wherein said polypeptide has a length that is less than 100 amino acids, preferably less than 100 amino acids, more preferably less than 90 amino acids, more preferably less than 80 amino acids, more preferably less than 70 amino acids, more preferably less than 75 amino acids, more preferably less than 70 amino acids, more preferably less than 65 amino acids, more preferably less than 60 amino acids, more preferably less than 55 amino acids.

In one aspect the invention concerns a method of treatment of cardiovascular diseases characterised by or resulting from hyperglycaemia, said method comprising administering to an individual in need thereof a therapeutically effective amount of an agent selected from the group consisting of:
a) an isolated polypeptide consisting of less than 100 amino acid residues and comprising:
   i) the amino acid sequence of SEQ ID NO: 1; or
   ii) a biologically active sequence variant of the amino acid sequence of i) wherein the variant has at least 70% sequence identity to said SEQ ID NO: 1,
   iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c).

In one aspect the invention concerns an isolated polypeptide for use in a method of treatment or prevention of cardiovascular diseases characterised by or resulting from hyperglycaemia and/or hyperinsulinaemia, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1
   wherein said polypeptide has a length that is less than 100 amino acids.

The sequence variant can be any biologically active variant wherein the biological activity is induction of glucose uptake in cells. Preferably the variant is a sequence variant of SEQ ID NO: 1 wherein any amino acid residue of SEQ ID NO: 1 has been altered to another amino acid residue, provided that no more than 15 amino acids have been so altered, such as wherein no more than 14 amino acids have been so altered, e.g. wherein no more than 13 amino acids have been so altered, such as wherein no more than 12 amino acids have been so altered, e.g. wherein no more than 11 amino acids have been so altered, such as wherein no more than 10 amino acids have been so altered, e.g. wherein no more than 9 amino acids have been so altered, such as wherein no more than 8 amino acids have been so altered, e.g. wherein no more than 7 amino acids have been so altered, such as wherein no more than 6 amino acids have been so altered, e.g. wherein no more than 5 amino acids have been so altered, such as wherein no more than 4 amino acids have been so altered, e.g. wherein no more than 3 amino acids have been so altered, such as wherein no more than 2 amino acids have been so altered, e.g. wherein no more than 1 amino acid has been so altered in relation to said SEQ ID NO: 1.

In one embodiment the polypeptide of the present invention has at least 60% sequence identity to the amino acid sequence of SEQ ID NO: 1, more preferably at least 70% sequence identity to the amino acid sequence of SEQ ID NO: 1, more preferably at least 75% sequence identity to the amino acid sequence of SEQ ID NO: 1, more preferably at least 80% sequence identity to the amino acid sequence of SEQ ID NO: 1, more preferably at least 85% sequence identity to the amino acid sequence of SEQ ID NO: 1, more preferably 90% sequence identity to the amino acid sequence of SEQ ID NO: 1, more preferably at least 95% sequence identity to the amino acid sequence of SEQ ID NO: 1, more preferably at least 98% sequence identity to the amino acid sequence of SEQ ID NO: 1, more preferably at least 99% sequence identity to the amino acid sequence of SEQ ID NO: 1.

In another embodiment, the In one embodiment the polypeptide of the present invention has at least 60% sequence identity to any one of the amino acid sequences of SEQ ID NO: 1 to 1035, more preferably at least 70% sequence identity to any one of the amino acid sequences of SEQ ID NO: 1 to 1035, more preferably at least 75% sequence identity to any one of the amino acid sequences of SEQ ID NO: 1 to 1035, more preferably at least 80% sequence identity to any one of the amino acid sequences of SEQ ID NO: 1 to 1035, more preferably at least 85% sequence identity to any one of the amino acid sequences of SEQ ID NO: 1 to 1035, more preferably 90% sequence identity to the amino acid sequence of to any one of the amino acid sequences of SEQ ID NO: 1 to 1035, more preferably at least 95% sequence identity to the amino acid sequence of to any one of the amino acid sequences of SEQ ID NO: 1 to 1035, more preferably at least 98% sequence identity to the amino acid sequence of to any one of the amino acid sequences of SEQ ID NO: 1 to 1035, more preferably at least 99% sequence identity to the amino acid sequence of to any one of the amino acid sequences of SEQ ID NO: 1 to 1035.

In one embodiment the polypeptide for use as defined herein consists essentially of any one of the amino acid sequences defined herein, such as consisting essentially of any one of SEQ ID NOs: 1 to 1035.

In one embodiment the polypeptide for use as defined herein consists of any one of the amino acid sequences defined herein, such as consisting of any one of SEQ ID NOs: 1 to 1035.

In one embodiment the polypeptide according to the present invention is a variant polypeptide, wherein in said variant any amino acid has been altered to provide a conservative substitution relative to the amino acid sequence of SEQ ID NO: 1.

In one embodiment the polypeptide according to the present invention comprises conserved amino acid residues at positions Ala 1, Glu 2, Tyr 3, His 4, Ala 5, Lys 6, Ala 7, Glu 9, Leu 11, Leu 14, Glu 16, Lys 17, Pro 20, Leu 22, Glu 23, Asp 24, Leu 25, Arg 26, Leu 29, Pro 31, Glu 34, Lys 37, Glu 45, Glu 46, Lys 49, Lys 50, Leu 51, Gln 54 relative to the amino acid sequence of SEQ ID NO:1.

In one embodiment the polypeptide according to the present invention comprises conserved amino acid residues at positions Glu 2, Tyr 3, Leu 11, Leu 14, Glu 16, Lys 17, Pro 20, Asp 24, Leu 29, Pro 31, Glu 34, Lys 37, Glu 45 relative to the amino acid sequence of SEQ ID NO:1.

In one embodiment, the polypeptide according to the present invention is not full length pre-pro-apoA-I. In another embodiment, the polypeptide according to the present invention is not full length pro-apoA-I. In yet another embodiment, the polypeptide according to the present invention is not full length mature apoA-I.

It is to be understood that the polypeptide exhibiting the biological activity of the present invention is a peptide derived from the C-terminal domain (i.e. AA190-243) of human apoA-I, such as SEQ ID NO: 1 or a fragment or variant thereof as defined herein.

Hyperlipidaemia encompasses several conditions of elevated blood lipid levels (mostly involving either elevated cholesterol “hypercholesterolaemia”, or elevated triglycerides “hyperglyceridaemia” or a combination of both). Hyperlipidaemia can be treated by administering the apoA-I peptide of the present invention since the peptide improve blood glucose control, inhibit lipolysis and thereby lower LDL cholesterol produced from the liver, which inhibit the aetiology of hyperlipidaemia (Solano et al (2006) Cardiol Rev. 14(3):125-35; Klop et al (2013) Nutrients 5: 1218-1240). Thus the present invention is useful for treating the following indications.

Diseases treatable by the present invention by regulating the glucose levels furthermore includes pure hypercholesterolaemia (including Familial hypercholesterolaemia, Fredrickson hyperlipoproteinaemia, type IIa, Hyperbetalipoproteinaemia, Hyperlipidaemia group A, Low-density-lipoprotein-type [LDL] hyperlipoproteinaemia); Pure hyperglyceridaemia (including endogenous hyperglyceridaemia, Fredrickson hyperlipoproteinaemia, type IV, Hyperlipidaemia, group B Hyperprebetalipoproteinaemia, Very-low-density-lipoprotein-type [VLDL] hyperlipoproteinaemia; Mixed hyperlipidaemia (including Broad- or floating-betalipoproteinaemia, Fredrickson hyperlipoproteinaemia, type IIb or III, Hyperbetalipoproteinaemia with prebetalipoproteinaemia, Hypercholesterolaemia with endogenous hyperglyceridaemia, Hyperlipidaemia, group C, Tubero-eruptive xanthoma, Xanthoma tuberosum); Hyperchylomicronaemia (including Fredrickson hyperlipoproteinaemia, type I or V, Hyperlipidaemia, group D, Mixed hyperglyceridaemia; and Other hyperlipidaemia (including Familial combined hyperlipidaemia Lipoprotein deficiency (including Abetalipoproteinaemia, High-density lipoprotein deficiency, Hypoalphalipoproteinaemia, Hypobetalipoproteinaemia (familial), Lecithin cholesterol acyltransferase deficiency, Tangier's disease).

In one embodiment, the polypeptide of any one of SEQ ID NOs: 1 to 1035 is not for use in the treatment of hyperlipidemia or hypercholesterolemia.

In another embodiment, the polypeptide of any one of SEQ ID NOs: 1 to 1035 is however for use in the treatment of cardiovascular other than hyperlipidemia or hypercholesterolemia.

Abnormal glucose tolerance test including chemical and latent diabetes, impaired glucose tolerance and prediabetes involves elevated blood glucose levels that without intervention will develop into type 2 diabetes. Lifestyle changes including healthy food and increased physical activity could potentially restore normal blood glucose levels. Impaired glucose tolerance and prediabetes can be reversed by administering the apoA-I peptide of the present invention because the peptide results in improved glucose metabolism that inhibits the aetiology of impaired glucose tolerance and prediabetes. Restored glucose metabolism at an early stage is desirable to prevent progress into manifest type 2 diabetes associated with increased cardiovascular diseases (Nichols et al (2007) Diabetes Care 30(2):228-233).

In one embodiment the polypeptide according to the present invention is for use in the treatment of a disease selected from the group consisting of disorders of lipoprotein metabolism and other lipidaemias; abnormal findings from blood; diseases of arteries, arterioles and capillaries; ischaemic and other heart diseases.

In one embodiment the disease treatable by the present invention is a disorder of lipoprotein metabolism and other lipidaemias such as a disorder of lipoprotein metabolism and other lipidaemias selected from the group consisting of pure hypercholesterolaemia such as familial hypercholesterolaemia; Fredrickson hyperlipoproteinaemia, type IIa; Hyperbetalipoproteinaemia; Hyperlipidaemia group A; and Low-density-lipoprotein-type [LDL] hyperlipoproteinaemia.

In one embodiment the disease treatable by the present invention is a disorder of lipoprotein metabolism and other lipidaemias selected from the group consisting of pure hyperglyceridaemia including endogenous hyperglyceridaemia; Fredrickson hyperlipoproteinaemia, type IV; Hyperlipidaemia, group B; hyperprebetalipoproteinaemia; and very-low-density-lipoprotein-type [VLDL] hyperlipoproteinaemia.

In one embodiment the disease treatable by the present invention is a disorder of lipoprotein metabolism and other lipidaemias selected from the group consisting of mixed hyperlipidaemia such as Broad- or floating-betalipoproteinaemia; Fredrickson hyperlipoproteinaemia, type IIb or III; Hyperbetalipoproteinaemia with prebetalipoproteinaemia; hypercholesterolaemia with endogenous hyperglyceridaemia; hyperlipidaemia, group C; tubero-eruptive xanthoma; and Xanthoma tuberosum.

In one embodiment the disease treatable by the present invention is a disorder of lipoprotein metabolism and other lipidaemias selected from the group consisting of hyperchylomicronaemia such as Fredrickson hyperlipoproteinaemia, type I or V; hyperlipidaemia, group D; and mixed hyperglyceridaemia.

In one embodiment the disease treatable by the present invention is a disorder of lipoprotein metabolism and other lipidaemias selected from the group consisting of other hyperlipidaemia such as familial combined hyperlipidaemia.

In one embodiment the disease treatable by the present invention is a disease of lipoprotein metabolism and other lipidaemias selected from the group consisting of lipoprotein deficiency such as abetalipoproteinaemia, high-density lipoprotein deficiency, hypoalphalipoproteinaemia; hypobetalipoproteinaemia (familial); lecithin cholesterol acyltransferase deficiency and Tangier's disease.

Atherosclerosis (including arteriolosclerosis, arteriosclerotic vascular disease, atheroma, degeneration (arterial, arteriovascular and vascular)) and atherosclerotic heart disease (including coronary (artery)) are indications involving thickening of the wall of arteries, along with stiffness and a loss of elasticity. Atherosclerosis, the most common form of arteriosclerosis, is a condition where arteries become narrowed and hardened due to an excessive build-up of plaque (consisting of LDL cholesterol and macrophages) around the artery wall, causing serious cardiovascular complications. Atherosclerosis can be treated and/or prevented by administering the apoA-I peptide of the present invention because the peptide 1) results in lowered glucose levels and thereby reduce one of the risk factors associated with atherosclerosis; 2) administration of the apoA-I peptide of the present invention can improve atherosclerosis by improving HDL-levels and thereby improve removal of cholesterol from macrophages, inhibition of oxidation of LDL, and by limiting the underlying inflammatory processes; 3) administration of the apoA-I peptide of the present invention can improve atherosclerosis by restoring insulin levels that will suppress lipolysis from adipose tissue and triglyceride rich lipoprotein particles and hence reduce levels of circulating fatty acids and following LDL levels which will reduce progression of atherosclerosis (Barter (2005) Eur Heart J. 7 (suppl F): F4-F8; Rader (2002) Am J of Cardiology, 90(8):62-70).

As the present invention in one embodiment is useful for preventing atherosclerosis, it is also useful in preventing ischaemic and other heart diseases such as angina pectoris, myocardial infarction, aortic stenosis and cardiomyopathy in metabolic diseases. Metabolic cardiomyopathies are commonly caused by altered energy metabolism. Cardio-myopathy in metabolic diseases can be treated by administering the apoA-I peptide of the present invention because the peptide results in improve glucose metabolism, lowered circulating fatty acids, decreased LDL cholesterol which improve myocardial glucose uptake and glycolytic flux, that inhibits the aetiology of cardiomyopathy associated with metabolic disease (Guertl et al. (2000) 81(6): 349-372; Witteles et al (2008) J Am Coll Cardiol. 51(2):93-102); van de Weijer et al (2011) 92: 10-18).

Thus in one embodiment the disease treatable by the present invention is a disease of arteries, arterioles and capillaries selected from the group consisting of atherosclerosis such as arteriolosclerosis; arteriosclerotic vascular disease; atheroma; arterial degeneration; arteriovascular degeneration and vascular degeneration.

In one embodiment the disease treatable by the present invention is a disease of arteries, arterioles and capillaries are selected from the group consisting of atherosclerotic heart disease such as coronary (artery) heart disease.

In one embodiment the disease treatable by the present invention is a disease belonging to the group ischaemic and other heart diseases selected from the group consisting of angina pectoris; myocardial infarction; aortic stenosis; and cardiomyopathy in metabolic diseases.

In one embodiment the disease treatable by the present invention is a cardiovascular disease characterised by non-normal lipid levels or a lipid containing deposition within body components. The therapeutic effect thus also relates to the treatment of a disease or condition characterised by non-normal lipid levels or a lipid containing deposition within body components, such as acute coronary syndrome, or atherosclerosis, or atherosclerotic plaques in blood vessels, or valvular stenosis, or septic shock, or angina pectoris, or myocardial infarction, or unstable angina pectoris, or arterial stenoses, or peripheral artery diseases (PAD), or carotis stenosis, or cerebral arterial stenosis, or coronary arterial stenosis, or vascular demencia, or restenosis, or vulnerable plaqueor, or amaurosis fugax.

### III. Administration and Formulation

ApoA-I derived polypeptides of the invention may be administered in any manner, which is medically acceptable. This may include injections, by parenteral routes such as intravenous, intravascular, intraarterial, subcutaneous, intramuscular, intratumor, intraperitoneal, intraventricular, intraepidural, intracranial or others as well as nasal, or topical. Slow release administration is also specifically included in the invention, by such means as depot injections or erodible implants.

Administration of apoA-I according to this invention may be achieved using any suitable delivery means, including but not limited to injection, either subcutaneously, intravenously, intra-arterially, intramuscularly, intrathecally or to other suitable site; pump (see, e.g., Annals of Pharmacotherapy, 27:912 (1993); Cancer, 41:1270 (1993); Cancer Research, 44:1698 (1984), incorporated herein by reference), microencapsulation (see, e.g., U.S. Pat. Nos. 4,352,883; 4,353,888; and 5,084,350, herein incorporated by reference),

slow release polymer implants (see, e.g., Sabel, U.S. Pat. No. 4,883,666, incorporated herein by reference),
encapsulated and unencapsulated cell grafts (see, e.g., U.S. Pat. Nos. 5,082,670 and 5,618,531, each incorporated herein by reference); and inhalation.

Administration may be by periodic injections of a bolus of the preparation, or may be made more continuous by intravenous or intraperitoneal administration from a reservoir which is external (e.g., an IV bag) or internal (e.g., a bioerodable implant, a bioartificial organ, or a colony of implanted apoA-I derived peptide producing cells). See, e.g., U.S. Pat. Nos. 4,407,957, 5,798,113, and 5,800,828, each incorporated herein by reference.

Localised delivery may be by such means as delivery via a catheter to one or more arteries. A further type of localised delivery comprises local delivery of gene therapy vectors, which are normally injected.

In a preferred embodiment of the present invention the administration is parenteral injection, preferably intravenous, subcutaneous, intramuscular, intracranial or intraperitoneal. Whilst it is possible for the compounds of the present invention to be administered as the raw chemical (e.g. polypeptide alone), it is preferred to present them in the form of a pharmaceutical formulation. The pharmaceutical formulations may be prepared by conventional techniques, e.g. as described in Remington: The Science and Practice of Pharmacy 2005, Lippincott, Williams & Wilkins.

The term “pharmaceutically acceptable carrier” means one or more organic or inorganic ingredients, natural or synthetic, with which the apoA-I derived peptide or polypeptide is combined to facilitate its application. A suitable carrier includes sterile saline although other aqueous and non-aqueous isotonic sterile solutions and sterile suspensions known to be pharmaceutically acceptable are known to those of ordinary skill in the art.

The compounds of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A liposome system may be any variety of unilamellar vesicles, multilamellar vesicles, or stable plurilamellar vesicles, and may be prepared and administered according to methods well known to those of skill in the art, for example in accordance with the teachings of U.S. Pat. Nos. 5,169,637, 4,762,915, 5,000,958 or 5,185,154. In addition, it may be desirable to express the novel polypeptides of this invention, as well as other selected polypeptides, as lipoproteins, in order to enhance their binding to liposomes. A recombinant apoA-I derived protein is purified, for example, from CHO cells by immunoaffinity chromatography or any other convenient method, then mixed with liposomes and incorporated into them at high efficiency. The liposome-encapsulated protein may be tested in vitro for any effect on stimulating cell growth.

Where slow-release administration of an apoA-I polypeptide is desired in a formulation with release characteristics suitable for the treatment of any disease or disorder requiring administration of an apoA-I polypeptide, microencapsulation of an apoA-I derived peptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The slow-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

In one embodiment of the present invention a composition comprising apoA-I derived peptides or polypeptides is contemplated. The composition may comprise an isolated polypeptide as described herein, an isolated nucleic acid as described herein, an expression vector encoding an apoA-I derived peptide as described herein, or a cell line expressing apoA-I derived peptides as described herein.

In one embodiment the invention concerns an apoA-I derived peptide or polypeptide for use according to the present invention, wherein the polypeptide is administered or adapted for administration enterally, topically, parenterally or as part of a sustained release implant.

In one embodiment the invention concerns an apoA-I derived peptide or polypeptide for use according to the present invention, wherein the parenteral administration is intravenous, subcutaneous, intramuscular, intracranial or intraperitoneal.

In one embodiment the invention concerns an apoA-I derived peptide or polypeptide for use according to the present invention, wherein the enteral administration is oral, rectal, or buccal.

In one embodiment the invention concerns an apoA-I derived peptide or polypeptide for use according to the present invention, wherein the topical administration is dermal, epicutaneous, vaginal, intravesical, pulmonary, intranasal, intratracheal or as eye drops.

In one embodiment the invention concerns an apoA-I derived peptide or polypeptide for use according to the present invention, wherein the polypeptide is administered or adapted for administration subcutaneously or intravenously.

In one embodiment said administration is repeated daily, and in another embodiment said administration is repeated at least 1-3 times weekly, such as 2-5 times weekly, such as 3-6 times weekly.

In one aspect, the present invention concerns a pharmaceutical composition comprising an isolated polypeptide according to the present invention, as defined herein.

In one embodiment the pharmaceutical composition comprises a pharmaceutical ingredient in combination with another composition for the treatment of metabolic diseases, or for the prophylactic treatment of a mammal facing the risk of developing a metabolic disease.

In one embodiment the compound or composition is for the treatment of metabolic diseases, or for the prophylactic treatment of a mammal facing the risk of developing a metabolic disease is combined with a diabetes drug selected from the group of insulin (or a derivative of insulin), metformin or the like.

In one embodiment, the pharmaceutical composition further comprises a second active ingredient for treatment of metabolic disease and/or cardiovascular disease. In one embodiment said second active ingredient is a compound used for the treatment of metabolic diseases, or for the prophylactic treatment of a mammal facing the risk of developing a metabolic disease.

In one embodiment said second active ingredient is a compound used for treatment of diabetes. In one embodiment said second active ingredient is a compound selected from the group consisting of insulin, a derivative of insulin, metformin or derivatives thereof.

In one embodiment said second active ingredient is a compound selected from the group consisting of diuretics, angiotensin-converting enzyme (ACE) inhibitors, beta blockers, blood thinners such as aspirin; and cholesterol-lowering drugs such as statins or fibrates.

In one embodiment said second active ingredient is a compound selected from the group consisting of Insulin, Exenatide, Exenatide Extended Release, Liraglutide, Pramlintide; Sulfonylureas, Biguanides, Meglitinides, Thiazolidinediones, DPP-4 inhibitors, SGLT2 Inhibitors, Alpha-glucosidase and Bile Acid Sequestrants inhibitors.

### IV. Dosages

Various dosing regimes for systemic administration are contemplated. In one embodiment, methods of administering to a subject a formulation comprising an apoA-I derived peptide or polypeptide according to the present invention include administering said peptide or polypeptide at a dosage of between 1 μg/kg and 10,000 μg/kg body weight of the subject, per dose. In another embodiment, the dosage is between 1 μg/kg and 7,500 μg/kg body weight of the subject, per dose. In a further embodiment, the dosage is between 1 μg/kg and 5,000 μg/kg body weight of the subject, per dose. In a different embodiment, the dosage is between 1 μg/kg and 2,000 μg/kg body weight of the subject, per dose. In yet another embodiment, the dosage is between 1 μg/kg and 1,000 μg/kg body weight of the subject, per dose. In yet another embodiment, the dosage is between 1 μg/kg and 700 μg/kg body weight of the subject, per dose. In a more preferable embodiment, the dosage is between 5 μg/kg and 500 μg/kg body weight of the subject, per dose. In a most preferable embodiment, the dosage is between 10 μg/kg and 100 μg/kg body weight of the subject, per dose. In a preferred embodiment the subject to be treated is human.

In one embodiment the polypeptide according to the present invention is administered or adapted for administration in dosages of 1 μg/kg-10,000 μg/kg body weight, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg body weight.

Guidance as to particular dosages and methods of delivery is provided in the literature; see, for example, WO 02/78730 and WO 07/100898. Guidance to the calculation of the human equivalent dosages based on dosages used in animal experiments is provided in Reagan-Shaw et al., FASEB J, 22, 659-661 (2007).

The dose administered must be carefully adjusted to the age, weight and condition of the individual being treated, as well as the route of administration, dosage form and regimen, and the result desired, and the exact dosage should be determined by the practitioner.

In one embodiment of the present invention the administration is repeated daily, such as 1-8 times daily, such as 2-5 times daily. In another embodiment the administration is repeated at least 1-3 times weekly, such as 2-5 times weekly, such as 3-6 times weekly, once every three days, once every four days, once every five days, once every six days, or once every 7 days.

In other embodiments, the apoA-I derived peptide or polypeptide according to the present invention is administered at relatively long dosage interval. A relatively long dosage interval is intended to include at least 2 days between dosages, such as at least 3 days between dosages, for example 2 dosages per week. More preferably the long dosages intervals is at least one week, such as at least 2 weeks, more preferably at least 3 weeks, such as at least 4 weeks, or at least one month.

In one embodiment the polypeptide according to the present invention is administered daily.

In another embodiment the administration of the polypeptide according to the invention is repeated at least 1-3 times weekly, such as 2-5 times weekly, such as 3-6 times weekly.

Expressed in a different way the dosage intervals are so long that following one dosage of the apoA-I derived polypeptide, the polypeptide is no longer detectable in the serum of the subject to be treated when the next dosage is administered. In another embodiment the blood serum level is below 10 ng/mL, such as below 5 ng/mL, more preferably below 1 ng/mL, such as below 0.5 ng/mL, for example below 0.1 ng/mL.

In some embodiments, the initial administration of the apoA-I derived peptide is, e.g., twice daily, daily, once every two days, once every three days, or once every four days. This dosing schedule may be maintained e.g., for 2, 3, 4, 5, 6, 7, 9, 11, 14, 21 days, or more. After completion of this dosing schedule, the apoA-I derived peptide can be administered less frequently, e.g., as described above.

### V. apoA-I Derived Peptides

The present invention relates to the use of polypeptides and peptides derived from the C-terminal domain (AA190-243) of apolipoprotein A-I (apoA-I), as well as and polynucleotides encoding said protein, in the treatment of diseases characterised by or resulting from hyperglycaemia.

ApoA-I is a protein that in humans is encoded by the APOA1 gene. It has a specific role in lipid metabolism. ApoA-I is the major protein component of high density lipoprotein (HDL) in plasma. Chylomicrons secreted from the intestinal enterocyte also contain apoA-I but it is quickly transferred to HDL in the bloodstream. The protein is a cofactor for lecithin cholesterolacyltransferase (LCAT) which is responsible for the formation of most plasma cholesteryl esters. ApoA-I was also isolated as a prostacyclin (PGI2) stabilizing factor, and thus may have an anticlotting effect.

In one aspect the present invention concerns an isolated polypeptide for use in a method of treatment or prevention of diseases characterised by hyperglycaemia and/or insulin resistance, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1,
   wherein said polypeptide has a length that is less than 100 amino acids, and wherein said biological activity is induction of glucose uptake in cells.

Variants can differ from naturally occurring apoA-I peptides (e.g. SEQ ID NO: 1), in amino acid sequence or in ways that do not involve sequence, or in both ways. Variants in amino acid sequence (“sequence variants”) are produced when one or more amino acids in naturally occurring apoA-I peptide is substituted with a different natural amino acid, an amino acid derivative or non-native amino acid. Particularly preferred variants include naturally occurring apoA-I, or biologically active fragments of the C-terminal domain (AA190-243) of naturally occurring apoA-I peptide, whose sequences differ from the wild type sequence by one or more conservative and/or semi-conservative amino acid substitutions, which typically have minimal influence on the secondary and tertiary structure and hydrophobic nature of the protein or peptide. Variants may also have sequences, which differ by one or more non-conservative amino acid substitutions, deletions or insertions, which do not abolish the biological activity of the C-terminal domain (AA190-243) of apoA-I.

Substitutions within the following group (Clustal W, ‘strong’ conservation group) are to be regarded as conservative substitutions within the meaning of the present invention -S,T,A; N,E,Q,K; N,H,Q,K; N,D,E,Q; Q,H,R,K; M,I,L,V; M,I,L,F; H,Y; F,Y,W.

Substitutions within the following group (Clustal W, ‘weak’ conservation group) are to be regarded as semi-conservative substitutions within the meaning of the present invention

### -C,S,A; A,T,V; S,A,G; S,T,N,K; S,T,P,A; S,G,N,D; S,N,D,E,Q,K; N,D,E,Q,H,K; N,E,Q,H,R,K; V,L,I,M; H,F,Y.

Other variants within the invention are those with modifications which increase peptide stability. Such variants may contain, for example, one or more nonpeptide bonds (which replace the peptide bonds) in the peptide sequence. Also included are: variants that include residues other than naturally occurring L-amino acids, such as D-amino acids or non-naturally occurring or synthetic amino acids such as beta or gamma amino acids and cyclic variants. Incorporation of D-amino acids instead of L-amino acids into the polypeptide may increase its resistance to proteases. See, e. g., U.S. Pat. No. 5,219,990. Splice variants are specifically included in the invention.

In one embodiment, the polypeptide is a naturally occurring allelic variant of the sequence selected from the group consisting of SEQ ID No. 1 to 1035. This polypeptide may comprise an amino acid sequence that is the translation of a nucleic acid sequence differing by a single nucleotide from a nucleic acid sequence encoding SEQ ID NOs. 1 to 1035.

In one embodiment the polypeptide is a variant polypeptide as described herein, in one embodiment comprises a polypeptide wherein any amino acid specified in the chosen sequence is changed to provide a conservative substitution.

Non-sequence modifications may include, for example, in vivo or in vitro chemical derivatisation of portions of naturally occurring apoA-I, as well as acetylation, methylation, phosphorylation, carboxylation, PEG-ylation, or glycosylation. Just as it is possible to replace substituents of the protein, it is also possible to substitute functional groups, which are bound to the protein with groups characterised by similar features. Such modifications do not alter primary sequence. These will initially be conservative, i.e., the replacement group will have approximately the same size, shape, hydrophobicity and charge as the original group.

One approach to improve the efficacy of a therapeutic protein such as the apoA-I derived peptide fragments of the present invention is to increase its serum persistence, thereby allowing higher circulating levels, and/or allowing circulating levels to be present for a longer time thereby providing higher exposure (AUC), less frequent administration and reduced doses.

In determining bioequivalence, for example, between two products such as a commercially-available product and a candidate drug, pharmacokinetic studies are conducted whereby each of the preparations are administered in a cross-over study to volunteer subjects, generally healthy individuals but occasionally in patients. Serum/plasma samples are obtained at regular intervals and assayed for parent drug (or occasionally metabolite) concentration. Occasionally, blood concentration levels are neither feasible nor possible to compare the two products, then pharmacodynamic endpoints rather than pharmacokinetic endpoints are used for comparison. For a pharmacokinetic comparison, the plasma concentration data are used to assess key pharmacokinetic parameters such as area under the curve (AUC), peak concentration (Cₘₐₓ), time to peak concentration (Tₘₐₓ), and absorption lag time (t_{lag}). Testing can be conducted at several different doses, especially when the drug displays non-linear pharmacokinetics.

In addition to data from bioequivalence studies, other data may need to be submitted to meet regulatory requirements for bioequivalence. Such evidence may include analytical method validation and/or in vitro-in vivo correlation studies (IVIVC).

In one particular embodiment, the agent of the invention, such as the polypeptide of the invention is modified in order to provide higher exposure (AUC), less frequent administration and reduced doses.

In another embodiment, the agent of the invention, such as the polypeptide of the invention is modified in order to increase its half-life when administered to a patient, in particular its plasma half-life. In particular, the agent, such as the polypeptide is modified in order to increase its plasma half-life. A number of methods are available in the art for modification of peptide drugs in order to increase its half-life, and such methods of the art can be employed for modification of the apoA-I derived peptides of the present invention and variants thereof. Short plasma half-life times are often caused by fast renal clearance as well as enzymatic degradation occurring during systemic circulation. Modifications of the peptide/protein can lead to prolonged plasma half-life times. Increased half-life can for example be obtained by shortening the overall amino acid amount of the polypeptide.

Exopeptidases is a prominent group of proteolytic enzymes occurring in plasma, liver and kidney, which affect therapeutic peptides and proteins. Thus, modifications of either or both of the peptide drug termini in many cases increase enzymatic stability, and thus plasma half-life. Thus, in one approach, one or more additional compounds are coupled to a polypeptide of the present invention, in order to increase its plasma half-life. In one embodiment, the terminal modification is N-acetylation and/or C-amidation. In another such embodiment, The N and/or C-terminus is conjugated to polyethylenglycol (PEG) compounds. One specific modification of the polypeptide is the dual modification of N-terminal palmitoyl and C-terminal PEGylation. A head-to-tail cyclization of the polypeptide drug by the formation of an amide bond between C- and N-terminus is also possible in order to prevent exopeptidase caused degradation of the apoA-I derived peptide.

In another embodiment, increased plasma half-life is obtained by replacement of one or more amino acids, which are known to be susceptible to enzymatic cleavage, thereby letting the polypeptide escape proteolytic degradation. For example, one or more L-amino acids could be substituted with D-amino acids at one or both polypeptide termini, and/or within the polypeptide in order to avoid degradation, and thereby increase plasma half-life.

Increased half-life of the polypeptide of the invention can also be obtained by co-administration of the polypeptide with one or more specific enzyme inhibitors. Such enzyme inhibitors could be included in the kit-of-parts of the invention. In yet another approach, increased half-life could be obtained by increasing the molecular mass of the apoA-I derived peptides of the invention.

As a general rule, substances with a molecular mass below 5 kDa which are not bound to plasma proteins are excreted via the renal route, whereas molecules with a molecular mass over 50 kDa cannot or only in very small amounts be found in the glomerular ultrafiltrate. Accordingly, a main reason for short peptide and protein half-life time beside enzymatic degradation is their fast renal excretion. Therefore, half-life time can be prolonged by increasing the polypeptide drug size. Furthermore, a synergistic effect may be given by additional enzyme inhibition. Beside chemical modification of N- and C-termini which is an effective way to inhibit exopeptidases and replacement of labile amino acids, PEGylation allows to specifically protect endangered termini and furthermore increases molecular mass. In addition, PEGylation within the drug molecule expectedly leads to improved enzymatic stability mediated by a steric hindrance of proteolytic enzymes.

Poly(ethyleneglycol) (PEG) exhibits several beneficial properties: high water solubility, high mobility in solution, lack of toxicity and immunogenicity and ready clearance from the body. Very often these properties are transferred to PEG-protein or PEG-peptide conjugates. The extent of these feature are dependent on the molecular weight of the attached PEG.

Also polymers of N-acetylneuraminic acid (polysialic acids) may be used as conjugates to a polypeptide of the invention. Polysialic acids are naturally occurring, biodegradable, highly hydrophilic compounds which have no known receptors in the human body. PEGylation and sialyation prolong half-life time by a combination of two mechanisms—improvement of enzymatic stability and decrease of renal excretion by increasing molecular mass.

Albumin is known to have a long plasma half-life and because of this property it has been used in drug delivery in order to increase half-life of drugs. For this purpose albumin has been conjugated to such pharmaceutical compounds. Especially suitable is coupling to the free cysteine residue on the albumin molecule (Cys 34), e.g. by methods described in WO2010092135, especially the methods using PDPH (3-(2-pyridyldithio) propionyl hydrazide) to link albumin to an apoA-I derived peptide of the invention including fragments thereof via a hydrazone link to the apoA-I polypeptide. Another coupling technology is described by Neose (see eg US2004/0126838) using enzymatic glycoconjugation. This technology can be used to link e.g. albumin to an apoA-I polypeptide of the invention using a suitable linker.

In certain embodiments the present invention concerns a long-acting modified apoA-I derived peptides wherein said modified polypeptide comprises a mammalian apoA-I derived peptide or analog thereof linked to a pharmaceutically acceptable molecule, e.g. human apoA-I derived peptide linked to, e.g. fused to, albumin, or fused to a fatty acid of suitable length, or fused to an Fc fragment of a mammalian antibody, or a variant of an Fc fragment of a mammalian antibody or conjugated to an acylation group or PEG, that in some embodiments provides an in vivo plasma half-life of the mammalian apoA-I derived peptide or analog thereof, or the modified apoA-I derived peptide which is from 2 to 48 hours or longer, typically from 4 to 28 hours, such as 6-8 hours in a mammal.

The creation of fusion proteins comprised of immunoglobulin constant regions linked to a protein of interest, or fragment thereof, has been described (see, e.g., U.S. Pat. Nos. 5,155,027, 5,428,130, 5,480,981, and 5,808,029). These molecules usually possess both the biological activity associated with the linked molecule of interest as well as the effector function, or some other desired characteristic, associated with the immunoglobulin constant region. Fusion proteins comprising an Fc portion of an immunoglobulin can bestow several desirable properties on a fusion protein including increased stability, increased serum half-life (see Capon et al. (1989) Nature 337:525) as well as binding to Fc receptors such as the neonatal Fc receptor (FcRn) (U.S. Pat. Nos. 6,086,875, 6,030,613, and 6,485,726).

In one embodiment the moiety resulting in increased half-life is a multifunctional moiety, such as bi- or trifunctional, which may be covalently linked to one or more apoA-I derived peptides, and covalently linked to one or more pharmaceutically acceptable molecule(s) so as to create the modified apoA-I derived peptide. The linker may be stabile which means that no significant chemical reactions, e.g. hydrolysis, occurs at physiological conditions (e.g. temperature of 37° C. and pH 7.4) over the time period of the treatment. This can be determined by stability studies known in the art. The linker may be a chemical linker meaning that it is generated by organic chemistry outside a living cell. The linker may be a sugar moiety, such as a glycosylation on a protein, or may be chemically prepared and used to link the apoA-I derived peptide molecule, and a second pharmaceutically acceptable molecule such as PEG variants, albumin, fatty acids or antibodies or antibody fragments such as Fc fragments.

In one embodiment, the apoA-I derived peptide of the invention is coupled to an immunoglobulin-Fc such as IgG-Fc.

The apoA-I derived peptide of the present invention may optionally comprise at least one peptide linker. In one embodiment, the linker is comprised of amino acids linked together by peptide bonds, wherein the amino acids are selected from the twenty naturally occurring amino acids. In various embodiments the linker can comprise 1-5 amino acids, 1-10 amino acids, 1-20 amino acids, 10-50 amino acids, 50-100 amino acids, or 100-200 amino acids. In one embodiment the amino acids are selected from glycine, alanine, proline, asparagine, glutamine, and lysine. In one embodiment a linker is made up of a majority of amino acids that are sterically unhindered, such as glycine and alanine.

In one embodiment the peptide linker has at least 1 amino acid, such as from 1-200 amino acids, typically 1-50 amino acids wherein the amino acids are selected from the twenty naturally occurring amino acids. Typically, the peptide linker has from 1-40 amino acids, such as from 1-30, such as from 1-20, such as from 1-10 amino acids. In a further embodiment the peptide linker is selected from a linker made up of amino acids selected from glycine, alanine, proline, asparagine, glutamine, and lysine. Typically, the peptide linker is made up of a majority of amino acids that are sterically unhindered, such as glycine and alanine.

The antibody, antibody fragment, albumin, fatty acid or any other one of the half-life extending can be conjugated to apoA-I derived peptides of the invention via any suitable linker or linker region. The linker may be a disulphide bridge, such as a —S—S— bond between two cysteine (Cys) amino acid residues in each of the apoA-I, and the pharmaceutically acceptable molecule. The linker may be a fused linker meaning that apoA-I derived peptide can be expressed in a living cell as one polypeptide or protein. The linker may be a hydrophilic linker that separates an apoA-I derived peptide and a pharmaceutically acceptable molecule with a chemical moiety, which comprises at least 5 non-hydrogen atoms where 30-50% of these are either N or O. The linker may be hydrolysable as described in U.S. Pat. No. 6,515,100, U.S. Pat. No. 7,122,189, U.S. Pat. No. 7,700,551, WO2004/089280, WO2006/138572 and WO2009/095479. Typical compounds useful as linkers in the present invention include those selected from the group having dicarboxylic acids, malemido hydrazides, PDPH, SPDP, LC-SPDP, GMBS, carboxylic acid hydrazides, and small peptides. More specific examples of compounds useful as linkers, according to the present invention, include: (a) dicarboxylic acids such as succinic acid, glutaric acid, and adipic acid; (b) maleimido hydrazides such as N-[maleimidocaproic acid]hydrazide (EMCH), N-[maleimidopropionic acid]hydrazide (MPH or BMPH), 4-[N-maleimidomethyl]cyclohexan-1-carboxylhydrazide, and N-[k-maleimidoundcanoic acid]hydrazide (KMUH), 4-(4-N-MaleimidoPhenyl)butyric acid Hydrazide (MPBH); (c) NHS-3-maleimidopropionate Succinimide ester (MPS-EDA); (d) PDPH linkers such as (3-[2-pyridyldithio]propionyl hydrazide) conjugated to sulfurhydryl reactive protein; (e) N-Succinimidyl 3-(2-pyridyldithio)-propionate (SPDP), (f) Succinimidyl 6-(3-[2-pyridyldithio]-propionamido)hexanoate (LC-SPDP), (g) N-(v-Maleimidobutyryloxy)succinimide ester (GMBS), and (h) carboxylic acid hydrazides selected from 2-5 carbon atoms. Other non-peptide linkers are also possible. For example, alkyl linkers such as —NH—(CH2)m-C(0)-, wherein m is an integer selected from 2-20, could be used. These alkyl linkers may further be substituted by any non-sterically hindering group such as lower alkyl (e.g., C1 to C6) lower acyl, halogen (e.g., Cl, Br, I, F), CN, NH2, phenyl, etc. An exemplary non-peptide linker is a PEG linker. Additional linkers useful according to the present invention are described in U.S. Pat. No. 6,660,843.

Different techniques for linking two or more molecules together, such as apoA-I derived peptide and the pharmaceutically acceptable molecule, and optionally via a multifunctional linker, such as bifunctional linker, are available in the prior art, and a suitable reference here is WO01/58493, including all relevant documents listed and cited therein.

In one embodiment the apoA-I derived peptide of the invention is modified in order to increase its half-life when administered to a patient, in particular its plasma half-life. The modification may be in the form of a moiety conjugated to the agent of the invention, thus generating a moiety-conjugated agent, wherein said moiety-conjugated agent has a plasma and/or serum half-life being longer than the plasma and/or serum half-life of the non-moiety conjugated agent. In one such embodiment the moiety conjugated to the agent is one or more type of moieties selected from the group consisting of albumin and variants thereof, fatty acids, polyethylene glycol (PEG), acylation groups, antibodies and antibody fragments. The conjugation of the moiety to the polypeptide of the invention may be to any suitable amino acid residue (backbone or side chain) of the polypeptide of the invention. The moiety may also be conjugated to polypeptide of the invention by a linker.

In one embodiment the moiety conjugated to the polypeptide according to the present invention is a moiety which facilitates transport across the blood brain barrier (BBB). An example of such a cross-BBB transport facilitator is an antibody from a camelid species. Camelids such as dromedaries, camels, llamas, alpacas, vicuñas, and guanacos have single-chain antibodies capable of crossing the BBB.

Many amino acids, including the terminal amino acids, may furthermore be modified in a given polypeptide, either by natural processes such as glycosylation and other posttranslational modifications, or by chemical modification techniques which are well known in the art. Among the known modifications which may be present in polypeptides of the present invention are, to name an illustrative few, acetylation, acylation, ADP-ribosylation, amidation, covalent attachment of flavin, covalent attachment of a heme moiety, covalent attachment of a polynucleotide or polynucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance, I. E. Creighton, Proteins-Structure and Molecular Properties, 2nd Ed., W. H. Freeman and Company, New York, 1993. Many detailed reviews are available on this subject, such as, for example, those provided by Wold, F., in Posttranslational Covalent Modification of Proteins, B. C. Johnson, Ed., Academic Press, New York, pp 1-12, 1983; Seifter et al., Meth. Enzymol. 182: 626-646, 1990 and Rattan et al., Protein Synthesis: Posttranslational Modifications and Aging, Ann. N.Y. Acad. Sci. 663: 48-62, 1992.

In addition, the protein may comprise a protein tag to allow subsequent purification and optionally removal of the tag using an endopeptidase. The tag may also comprise a protease cleavage site to facilitate subsequent removal of the tag. Non-limiting examples of affinity tags include a polyhis tag, a GST tag, a HA tag, a Flag tag, a C-myc tag, a HSV tag, a V5 tag, a maltose binding protein tag, a cellulose binding domain tag. Preferably for production and purification, the tag is a polyhistag. Preferably, the tag is in the C-terminal portion of the protein.

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

The modifications that occur in a polypeptide often will be a function of how it is made. For polypeptides made by expressing a cloned gene in a host, for instance, the nature and extent of the modifications in large part will be determined by the host cell's posttranslational modification capacity and the modification signals present in the polypeptide amino acid sequence. For instance, glycosylation often does not occur in bacterial hosts such as E. coli. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation, inter alia. Similar considerations apply to other modifications.

It will be appreciated that the same type of modification may be present to the same or varying degree at several sites in a given polypeptide. Also, a given polypeptide may contain many types of modifications.

In general, as used herein, the term polypeptide encompasses all such modifications, particularly those that are present in polypeptides synthesized by expressing a polynucleotide in a host cell.

In one aspect the present invention concerns an isolated polypeptide consisting essentially of an amino acid sequence selected from the group consisting of the amino acid sequence of SEQ ID NO: 3, 4 or 6.

In a further aspect the invention concerns an isolated polypeptide consisting of an amino acid sequence selected from the group consisting of the amino acid sequence of SEQ ID NO: 3, 4 or 6.

In yet another aspect the invention concerns an isolated polypeptide consisting essentially of an amino acid sequence selected from the group consisting of the amino acid sequence of SEQ ID NOs: 9 to 1035.

In a further aspect the invention concerns an isolated polypeptide consisting of an amino acid sequence selected from the group consisting of the amino acid sequence of SEQ ID NOs: 9 to 1035.

In one embodiment any one amino acid residue in said amino acid sequence has been altered to the corresponding D-amino acid, and in another embodiment all amino acid residues in said amino acid sequence has been altered to the corresponding D-amino acids.

In one aspect the invention concerns a polypeptide derived from the sequence of the apoA-I comprising or consisting of the SEQ ID NO: 1 (i.e. the human sequence corresponding for the 54-mer peptide, C-terminal peptide 190-243).

In one embodiment the apoA-I derived sequence comprise or consist of a polypeptide sequence not exceeding SEQ ID NO: 1 of apoA-I.

In one embodiment the polypeptide is bound to a polypeptide, a molecule or a linker that may be a polypeptide other than apoA-I.

In one aspect the invention concerns a pharmaceutical composition comprising the polypeptide as defined herein.

In one embodiment the active ingredient of the medicament of the present invention is a subsequence of SEQ ID NO: 1.

In one embodiment one or more amino acid substitutions, additions or deletions is/are present, that further increases the solubility of the peptide.

In one embodiment one or more amino acid substitutions, additions or deletions of the peptide of SEQ ID NO: 1 keeps or increases the glucose uptake of a mammalian cell.

In one embodiment, one or more amino acid substitutions, additions or deletions is present that increases the expression of the apoA-I derived polypeptide in a recombinant host cell, or in an in vitro translation system, or production in chemical synthesis.

In one embodiment, the biologically active polypeptide is at least one selected from the group of SEQ ID No 1 to SEQ ID No 8.

### VI. apoA-I Derived Polynucleotides

The invention provides medical use of isolated genomic DNA and cDNA coding for the C-terminal domain (AA190-243) of apoA-I (SEQ ID NO: 1) and fragments thereof (SEQ ID NO: 2 to 1035). Generally the cDNA sequence is much shorter than the genomic sequences are more easily inserted into an appropriate expression vector and transduced/fected into a production cell or a human cell in vivo or ex vivo.

In addition, the nucleotide sequences of the invention include sequences, which are derivatives of these sequences. The invention also includes vectors, liposomes and other carrier vehicles, which encompass one of these sequences or a derivative of one of these sequences. The invention also includes proteins transcribed and translated from apoA-I cDNA, preferably human apoA-I cDNA, including but not limited to human apoA-I and derivatives and variants.

Codon optimised nucleic acid molecules for enhanced expression in selected host cells, including but not limited to E. coli, yeast species, Chinese Hamster, Baby Hamster, insect, fungus, and human are also contemplated.

Variant nucleic acids can be made by state of the art mutagenesis methods. Methods for shuffling coding sequences from human with those of mouse, rat or chimpanzee are also contemplated.

Variant nucleic acids made by exchanging amino acids present in human apoA-I with the amino acid present in mouse or rat apoA-I at the corresponding position, should this amino acid be different from the one present in human apoA-I.

In one aspect the present invention concerns an isolated polynucleotide which upon expression encodes the polypeptide defined herein, such as the polypeptide having the sequence of SEQ ID NOs: 3, 4 or 6.

### VII. Vectors

### Viral Vectors

Broadly, gene therapy seeks to transfer new genetic material to the cells of a patient with resulting therapeutic benefit to the patient. Such benefits include treatment or prophylaxis of a broad range of diseases, disorders and other conditions.

Ex vivo gene therapy approaches involve modification of isolated cells (including but not limited to stem cells and alpha, beta and gamma cells of the pancreas, neural and glial precursor cells, and foetal stem cells), which are then infused, grafted or otherwise transplanted into the patient. See, e.g., U.S. Pat. Nos. 4,868,116, 5,399,346 and 5,460,959. In vivo gene therapy seeks to directly target host patient tissue in vivo.

Viruses useful as gene transfer vectors include papovavirus, adenovirus, vaccinia virus, adeno-associated virus, herpesvirus, and retroviruses. Suitable retroviruses include the group consisting of HIV, SIV, FIV, EIAV, MoMLV. A further group of suitable retroviruses includes the group consisting of HIV, SIV, FIV, EAIV, CIV. Another group of preferred virus vectors includes the group consisting of alphavirus, adenovirus, adeno associated virus, baculovirus, HSV, coronavirus, Bovine papilloma virus, Mo-MLV, preferably adeno associated virus.

Preferred viruses within the field of gene therapy are lentiviruses and adeno-associated viruses. Both types of viruses can integrate into the genome without cell divisions, and both types have been tested in pre-clinical animal studies.

A lentivirus vector is a replication-defective lentivirus particle. Such a lentivirus particle can be produced from a lentiviral vector comprising a 5′ lentiviral LTR, a tRNA binding site, a packaging signal, a promoter operably linked to a polynucleotide signal encoding said fusion protein, an origin of second strand DNA synthesis and a 3′ lentiviral LTR.

Retroviral vectors are the vectors most commonly used in human clinical trials, since they carry 7-8 kb and since they have the ability to infect cells and have their genetic material stably integrated into the host cell with high efficiency. See, e.g., WO 95/30761; WO 95/24929. Oncovirinae require at least one round of target cell proliferation for transfer and integration of exogenous nucleic acid sequences into the patient. Retroviral vectors integrate randomly into the patient's genome. Retroviruses can be used to target stem cells.

Three classes of retroviral particles have been described; ecotropic, which can infect murine cells efficiently, and amphotropic, which can infect cells of many species. The third class includes xenotrophic retrovirus which can infect cells of another species than the species which produced the virus. Their ability to integrate only into the genome of dividing cells has made retroviruses attractive for marking cell lineages in developmental studies and for delivering therapeutic or suicide genes to cancers or tumors.

For use in human patients, the retroviral vectors must be replication defective. This prevents further generation of infectious retroviral particles in the target tissue—instead the replication defective vector becomes a “captive” transgene stable incorporated into the target cell genome. Typically in replication defective vectors, the gag, env, and pol genes have been deleted (along with most of the rest of the viral genome). Heterologous DNA is inserted in place of the deleted viral genes. The heterologous genes may be under the control of the endogenous heterologous promoter, another heterologous promoter active in the target cell, or the retroviral 5′ LTR (the viral LTR is active in diverse tissues). Typically, retroviral vectors have a transgene capacity of about 7-8 kb.

Replication defective retroviral vectors require provision of the viral proteins necessary for replication and assembly in trans, from, e.g., engineered packaging cell lines. It is important that the packaging cells do not release replication competent virus and/or helper virus. This has been achieved by expressing viral proteins from RNAs lacking the ψ signal, and expressing the gag/pol genes and the env gene from separate transcriptional units. In addition, in some 2. and 3. generation retriviruses, the 5′ LTR's have been replaced with non-viral promoters controlling the expression of these genes, and the 3′ promoter has been minimised to contain only the proximal promoter. These designs minimize the possibility of recombination leading to production of replication competent vectors, or helper viruses.

### Expression Vectors

Construction of vectors for recombinant expression of the apoA-I derived peptides of the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982). Expression vectors may be used for generating producer cells for recombinant production of the apoA-I peptides and polypeptides for medical use, and for generating therapeutic cells secreting apoA-I derived peptides for naked or encapsulated therapy.

Briefly, construction of recombinant expression vectors employs standard ligation techniques. For analysis to confirm correct sequences in vectors constructed, the genes are sequenced using, for example, the method of Messing, et al., (Nucleic Acids Res., 9: 309-, 1981), the method of Maxam, et al., (Methods in Enzymology, 65: 499, 1980), or other suitable methods which will be known to those skilled in the art.

Size separation of cleaved fragments is performed using conventional gel electrophoresis as described, for example, by Maniatis, et al., (Molecular Cloning, pp. 133-134, 1982).

For generation of efficient expression vectors, these should contain regulatory sequences necessary for expression of the encoded gene in the correct reading frame. Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds., The molecular biology of tumor viruses: RNA tumor viruses, Cold Spring Harbor Laboratory, (NY 1982)). Moloney murine leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

Promoter and enhancer regions of a number of non-viral promoters have also been described (Schmidt et al., Nature 314: 285 (1985); Rossi and deCrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, SV40 promoter, Elongation Factor 1 alpha promoter (EF1-alpha), RSV, CAG. Examples of inducible/repressible promoters include: Tet-On, Tet-Off, Rapamycin-inducible promoter, Mx1, Mo-MLV-LTR, progesterone, RU486.

A group of preferred promoters include CAG, CMV, human UbiC, JeT, SV40, RSV, Tet-regulatable promoter, Mo-MLV-LTR, Mx1, Mt1 and EF-1 alpha.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences may be used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Further expression enhancing sequences include but are not limited to Woodchuck hepatitis virus post-transcriptional regulation element, WPRE, SP163, CMV enhancer, and Chicken [beta]-globin insulator or other insulators.

In one aspect the present invention concerns a vector, such as a viral vector or an expression vector, comprising the polynucleotide capable of encoding a polypeptide according to any one of SEQ ID NOs: 1 to 1035.

### VIII. Host Cells

In one aspect the invention relates to isolated host cells genetically modified with the vector according to the invention.

The invention also relates to cells suitable for biodelivery of peptides derived from the C-terminal domain of apoA-I, via naked or encapsulated cells, which are genetically modified to overexpress the apoA-I derived polypeptides, and which can be transplanted to the patient to deliver the bioactive apoA-I polypeptide locally, e.g. in a malfunctioning pancreas. Such cells may broadly be referred to as therapeutic cells.

For ex vivo gene therapy, the preferred group of cells includes stem cells, muscle cells, hepatocytes, adipocytes and cells of the pancreas such as α cells, β cells and δ cells.

In one aspect, the present invention concerns a host cell comprising the polynucleotide capable of encoding the polypeptide according to any one of SEQ ID NOs: 1 to 1035, or the vector comprising said polynucleotide.

In one embodiment the cell is selected from the group consisting of stem cells, muscle cells, hepatocytes, adipocytes and cells of the pancreas such as α cells, β cells and δ cells.

In another embodiment the cell is selected from the group consisting of CHO, CHO-K1, HEI193T, HEK293, COS, HiB5, RN33b and BHK cells.

### Overview of Sequences

SEQ ID NO. 1: AA190-243
SEQ ID NO. 2: AA190-207
SEQ ID NO. 3: AA208-225
SEQ ID NO. 4: AA226-243
SEQ ID NO. 5: AA199-216
SEQ ID NO. 6: AA217-234
SEQ ID NO. 7: AA190-216
SEQ ID NO. 8: AA217-243
SEQ ID NO. 9: AA190-242
SEQ ID NO. 10: AA191-243
SEQ ID NO. 11: AA190-241
SEQ ID NO. 12: AA191-242
SEQ ID NO. 13: AA192-243
SEQ ID NO. 14: AA190-240
SEQ ID NO. 15: AA191-241
SEQ ID NO. 16: AA192-242
SEQ ID NO. 17: AA193-243
SEQ ID NO. 18: AA190-239
SEQ ID NO. 19: AA191-240
SEQ ID NO. 20: AA192-241
SEQ ID NO. 21: AA193-242
SEQ ID NO. 22: AA194-243
SEQ ID NO. 23: AA190-238
SEQ ID NO. 24: AA191-239
SEQ ID NO. 25: AA192-240
SEQ ID NO. 26: AA193-241
SEQ ID NO. 27: AA194-242
SEQ ID NO. 28: AA195-243
SEQ ID NO. 29: AA190-237
SEQ ID NO. 30: AA191-238
SEQ ID NO. 31: AA192-239
SEQ ID NO. 32: AA193-240
SEQ ID NO. 33: AA194-241
SEQ ID NO. 34: AA195-242
SEQ ID NO. 35: AA196-243
SEQ ID NO. 36: AA190-236
SEQ ID NO. 37: AA191-237
SEQ ID NO. 38: AA192-238
SEQ ID NO. 39: AA193-239
SEQ ID NO. 40: AA194-240
SEQ ID NO. 41: AA195-241
SEQ ID NO. 42: AA196-242
SEQ ID NO. 43: AA197-243
SEQ ID NO. 44: AA190-235
SEQ ID NO. 45: AA191-236
SEQ ID NO. 46: AA192-237
SEQ ID NO. 47: AA193-238
SEQ ID NO. 48: AA194-239
SEQ ID NO. 49: AA195-240
SEQ ID NO. 50: AA196-241
SEQ ID NO. 51: AA197-242
SEQ ID NO. 52: AA198-243
SEQ ID NO. 53: AA190-234
SEQ ID NO. 54: AA191-235
SEQ ID NO. 55: AA192-236
SEQ ID NO. 56: AA193-237
SEQ ID NO. 57: AA194-238
SEQ ID NO. 58: AA234-243
SEQ ID NO. 59: AA232-241
SEQ ID NO. 60: AA195-239
SEQ ID NO. 61: AA196-240
SEQ ID NO. 62: AA197-241
SEQ ID NO. 63: AA198-242
SEQ ID NO. 64: AA199-243
SEQ ID NO. 65: AA190-233
SEQ ID NO. 66: AA191-234
SEQ ID NO. 67: AA192-235
SEQ ID NO. 68: AA193-236
SEQ ID NO. 69: AA194-237
SEQ ID NO. 70: AA195-238
SEQ ID NO. 71: AA196-239
SEQ ID NO. 72: AA197-240
SEQ ID NO. 73: AA198-241
SEQ ID NO. 74: AA199-242
SEQ ID NO. 75: AA200-243
SEQ ID NO. 76: AA190-232
SEQ ID NO. 77: AA191-233
SEQ ID NO. 78: AA192-234
SEQ ID NO. 79: AA193-235
SEQ ID NO. 80: AA194-236
SEQ ID NO. 81: AA195-237
SEQ ID NO. 82: AA196-238
SEQ ID NO. 83: AA197-239
SEQ ID NO. 84: AA198-240
SEQ ID NO. 85: AA199-241
SEQ ID NO. 86: AA200-242
SEQ ID NO. 87: AA201-243
SEQ ID NO. 88: AA190-231
SEQ ID NO. 89: AA191-232
SEQ ID NO. 90: AA192-233
SEQ ID NO. 91: AA193-234
SEQ ID NO. 92: AA194-235
SEQ ID NO. 93: AA195-236
SEQ ID NO. 94: AA196-237
SEQ ID NO. 95: AA197-238
SEQ ID NO. 96: AA198-239
SEQ ID NO. 97: AA199-240
SEQ ID NO. 98: AA200-241
SEQ ID NO. 99: AA201-242
SEQ ID NO. 100: AA202-243
SEQ ID NO. 101: AA190-230
SEQ ID NO. 102: AA191-231
SEQ ID NO. 103: AA192-232
SEQ ID NO. 104: AA193-233
SEQ ID NO. 105: AA194-234
SEQ ID NO. 106: AA195-235
SEQ ID NO. 107: AA196-236
SEQ ID NO. 108: AA197-237
SEQ ID NO. 109: AA198-238
SEQ ID NO. 110: AA199-239
SEQ ID NO. 111: AA200-240
SEQ ID NO. 112: AA201-241
SEQ ID NO. 113: AA202-242
SEQ ID NO. 114: AA203-243
SEQ ID NO. 115: AA190-229
SEQ ID NO. 116: AA191-230
SEQ ID NO. 117: AA192-231
SEQ ID NO. 118: AA193-232
SEQ ID NO. 119: AA194-233
SEQ ID NO. 120: AA195-234
SEQ ID NO. 121: AA196-235
SEQ ID NO. 122: AA197-236
SEQ ID NO. 123: AA198-237
SEQ ID NO. 124: AA199-238
SEQ ID NO. 125: AA200-239
SEQ ID NO. 126: AA201-240
SEQ ID NO. 127: AA202-241
SEQ ID NO. 128: AA203-242
SEQ ID NO. 129: AA204-243
SEQ ID NO. 130: AA190-228
SEQ ID NO. 131: AA191-229
SEQ ID NO. 132: AA192-230
SEQ ID NO. 133: AA193-231
SEQ ID NO. 134: AA194-232
SEQ ID NO. 135: AA195-233
SEQ ID NO. 136: AA196-234
SEQ ID NO. 137: AA197-235
SEQ ID NO. 138: AA198-236
SEQ ID NO. 139: AA199-237
SEQ ID NO. 140: AA200-238
SEQ ID NO. 141: AA201-239
SEQ ID NO. 142: AA202-240
SEQ ID NO. 143: AA203-241
SEQ ID NO. 144: AA204-242
SEQ ID NO. 145: AA205-243
SEQ ID NO. 146: AA190-227
SEQ ID NO. 147: AA191-228
SEQ ID NO. 148: AA192-229
SEQ ID NO. 149: AA193-230
SEQ ID NO. 150: AA194-231
SEQ ID NO. 151: AA195-232
SEQ ID NO. 152: AA196-233
SEQ ID NO. 153: AA197-234
SEQ ID NO. 154: AA198-235
SEQ ID NO. 155: AA199-236
SEQ ID NO. 156: AA200-237
SEQ ID NO. 157: AA201-238
SEQ ID NO. 158: AA202-239
SEQ ID NO. 159: AA203-240
SEQ ID NO. 160: AA204-241
SEQ ID NO. 161: AA205-242
SEQ ID NO. 162: AA206-243
SEQ ID NO. 163: AA190-226
SEQ ID NO. 164: AA191-227
SEQ ID NO. 165: AA192-228
SEQ ID NO. 166: AA193-229
SEQ ID NO. 167: AA194-230
SEQ ID NO. 168: AA195-231
SEQ ID NO. 169: AA196-232
SEQ ID NO. 170: AA197-233
SEQ ID NO. 171: AA198-234
SEQ ID NO. 172: AA199-235
SEQ ID NO. 173: AA200-236
SEQ ID NO. 174: AA201-237
SEQ ID NO. 175: AA202-238
SEQ ID NO. 176: AA203-239
SEQ ID NO. 177: AA204-240
SEQ ID NO. 178: AA205-241
SEQ ID NO. 179: AA206-242
SEQ ID NO. 180: AA207-243
SEQ ID NO. 181: AA190-225
SEQ ID NO. 182: AA191-226
SEQ ID NO. 183: AA192-227
SEQ ID NO. 184: AA193-228
SEQ ID NO. 185: AA194-229
SEQ ID NO. 186: AA195-230
SEQ ID NO. 187: AA196-231
SEQ ID NO. 188: AA197-232
SEQ ID NO. 189: AA198-233
SEQ ID NO. 190: AA199-234
SEQ ID NO. 191: AA200-235
SEQ ID NO. 192: AA201-236
SEQ ID NO. 193: AA202-237
SEQ ID NO. 194: AA203-238
SEQ ID NO. 195: AA204-239
SEQ ID NO. 196: AA205-240
SEQ ID NO. 197: AA206-241
SEQ ID NO. 198: AA207-242
SEQ ID NO. 199: AA208-243
SEQ ID NO. 200: AA190-224
SEQ ID NO. 201: AA191-225
SEQ ID NO. 202: AA192-226
SEQ ID NO. 203: AA193-227
SEQ ID NO. 204: AA194-228
SEQ ID NO. 205: AA195-229
SEQ ID NO. 206: AA196-230
SEQ ID NO. 207: AA197-231
SEQ ID NO. 208: AA198-232
SEQ ID NO. 209: AA199-233
SEQ ID NO. 210: AA200-234
SEQ ID NO. 211: AA201-235
SEQ ID NO. 212: AA202-236
SEQ ID NO. 213: AA203-237
SEQ ID NO. 214: AA204-238
SEQ ID NO. 215: AA205-239
SEQ ID NO. 216: AA206-240
SEQ ID NO. 217: AA207-241
SEQ ID NO. 218: AA208-242
SEQ ID NO. 219: AA209-243
SEQ ID NO. 220: AA190-223
SEQ ID NO. 221: AA191-224
SEQ ID NO. 222: AA192-225
SEQ ID NO. 223: AA193-226
SEQ ID NO. 224: AA194-227
SEQ ID NO. 225: AA195-228
SEQ ID NO. 226: AA196-229
SEQ ID NO. 227: AA197-230
SEQ ID NO. 228: AA198-231
SEQ ID NO. 229: AA199-232
SEQ ID NO. 230: AA200-233
SEQ ID NO. 231: AA201-234
SEQ ID NO. 232: AA202-235
SEQ ID NO. 233: AA203-236
SEQ ID NO. 234: AA204-237
SEQ ID NO. 235: AA205-238
SEQ ID NO. 236: AA206-239
SEQ ID NO. 237: AA207-240
SEQ ID NO. 238: AA208-241
SEQ ID NO. 239: AA209-242
SEQ ID NO. 240: AA210-243
SEQ ID NO. 241: AA190-222
SEQ ID NO. 242: AA191-223
SEQ ID NO. 243: AA192-224
SEQ ID NO. 244: AA193-225
SEQ ID NO. 245: AA194-226
SEQ ID NO. 246: AA195-227
SEQ ID NO. 247: AA196-228
SEQ ID NO. 248: AA197-229
SEQ ID NO. 249: AA198-230
SEQ ID NO. 250: AA199-231
SEQ ID NO. 251: AA200-232
SEQ ID NO. 252: AA201-233
SEQ ID NO. 253: AA202-234
SEQ ID NO. 254: AA203-235
SEQ ID NO. 255: AA204-236
SEQ ID NO. 256: AA205-237
SEQ ID NO. 257: AA206-238
SEQ ID NO. 258: AA207-239
SEQ ID NO. 259: AA208-240
SEQ ID NO. 260: AA209-241
SEQ ID NO. 261: AA210-242
SEQ ID NO. 262: AA211-243
SEQ ID NO. 263: AA190-221
SEQ ID NO. 264: AA191-222
SEQ ID NO. 265: AA192-223
SEQ ID NO. 266: AA193-224
SEQ ID NO. 267: AA194-225
SEQ ID NO. 268: AA195-226
SEQ ID NO. 269: AA196-227
SEQ ID NO. 270: AA197-228
SEQ ID NO. 271: AA198-229
SEQ ID NO. 272: AA199-230
SEQ ID NO. 273: AA200-231
SEQ ID NO. 274: AA201-232
SEQ ID NO. 275: AA202-233
SEQ ID NO. 276: AA203-234
SEQ ID NO. 277: AA204-235
SEQ ID NO. 278: AA205-236
SEQ ID NO. 279: AA206-237
SEQ ID NO. 280: AA207-238
SEQ ID NO. 281: AA208-239
SEQ ID NO. 282: AA209-240
SEQ ID NO. 283: AA210-241
SEQ ID NO. 284: AA211-242
SEQ ID NO. 285: AA212-243
SEQ ID NO. 286: AA190-220
SEQ ID NO. 287: AA191-221
SEQ ID NO. 288: AA192-222
SEQ ID NO. 289: AA193-223
SEQ ID NO. 290: AA194-224
SEQ ID NO. 291: AA195-225
SEQ ID NO. 292: AA196-226
SEQ ID NO. 293: AA197-227
SEQ ID NO. 294: AA198-228
SEQ ID NO. 295: AA199-229
SEQ ID NO. 296: AA200-230
SEQ ID NO. 297: AA201-231
SEQ ID NO. 298: AA202-232
SEQ ID NO. 299: AA203-233
SEQ ID NO. 300: AA204-234
SEQ ID NO. 301: AA205-235
SEQ ID NO. 302: AA206-236
SEQ ID NO. 303: AA207-237
SEQ ID NO. 304: AA208-238
SEQ ID NO. 305: AA209-239
SEQ ID NO. 306: AA210-240
SEQ ID NO. 307: AA211-241
SEQ ID NO. 308: AA212-242
SEQ ID NO. 309: AA213-243
SEQ ID NO. 310: AA190-219
SEQ ID NO. 311: AA191-220
SEQ ID NO. 312: AA192-221
SEQ ID NO. 313: AA193-222
SEQ ID NO. 314: AA194-223
SEQ ID NO. 315: AA195-224
SEQ ID NO. 316: AA196-225
SEQ ID NO. 317: AA197-226
SEQ ID NO. 318: AA198-227
SEQ ID NO. 319: AA199-228
SEQ ID NO. 320: AA200-229
SEQ ID NO. 321: AA201-230
SEQ ID NO. 322: AA202-231
SEQ ID NO. 323: AA203-232
SEQ ID NO. 324: AA204-233
SEQ ID NO. 325: AA205-234
SEQ ID NO. 326: AA206-235
SEQ ID NO. 327: AA207-236
SEQ ID NO. 328: AA208-237
SEQ ID NO. 329: AA209-238
SEQ ID NO. 330: AA210-239
SEQ ID NO. 331: AA211-240
SEQ ID NO. 332: AA212-241
SEQ ID NO. 333: AA213-242
SEQ ID NO. 334: AA214-243
SEQ ID NO. 335: AA190-218
SEQ ID NO. 336: AA191-219
SEQ ID NO. 337: AA192-220
SEQ ID NO. 338: AA193-221
SEQ ID NO. 339: AA194-222
SEQ ID NO. 340: AA195-223
SEQ ID NO. 341: AA196-224
SEQ ID NO. 342: AA197-225
SEQ ID NO. 343: AA198-226
SEQ ID NO. 344: AA199-227
SEQ ID NO. 345: AA200-228
SEQ ID NO. 346: AA201-229
SEQ ID NO. 347: AA202-230
SEQ ID NO. 348: AA203-231
SEQ ID NO. 349: AA204-232
SEQ ID NO. 350: AA205-233
SEQ ID NO. 351: AA206-234
SEQ ID NO. 352: AA207-235
SEQ ID NO. 353: AA208-236
SEQ ID NO. 354: AA209-237
SEQ ID NO. 355: AA210-238
SEQ ID NO. 356: AA211-239
SEQ ID NO. 357: AA212-240
SEQ ID NO. 358: AA213-241
SEQ ID NO. 359: AA214-242
SEQ ID NO. 360: AA215-243
SEQ ID NO. 361: AA190-217
SEQ ID NO. 362: AA191-218
SEQ ID NO. 363: AA192-219
SEQ ID NO. 364: AA193-220
SEQ ID NO. 365: AA194-221
SEQ ID NO. 366: AA195-222
SEQ ID NO. 367: AA196-223
SEQ ID NO. 368: AA197-224
SEQ ID NO. 369: AA198-225
SEQ ID NO. 370: AA199-226
SEQ ID NO. 371: AA200-227
SEQ ID NO. 372: AA201-228
SEQ ID NO. 373: AA202-229
SEQ ID NO. 374: AA203-230
SEQ ID NO. 375: AA204-231
SEQ ID NO. 376: AA205-232
SEQ ID NO. 377: AA206-233
SEQ ID NO. 378: AA207-234
SEQ ID NO. 379: AA208-235
SEQ ID NO. 380: AA209-236
SEQ ID NO. 381: AA210-237
SEQ ID NO. 382: AA211-238
SEQ ID NO. 383: AA212-239
SEQ ID NO. 384: AA213-240
SEQ ID NO. 385: AA214-241
SEQ ID NO. 386: AA215-242
SEQ ID NO. 387: AA216-243
SEQ ID NO. 388: AA191-217
SEQ ID NO. 389: AA192-218
SEQ ID NO. 390: AA193-219
SEQ ID NO. 391: AA194-220
SEQ ID NO. 392: AA195-221
SEQ ID NO. 393: AA196-222
SEQ ID NO. 394: AA197-223
SEQ ID NO. 395: AA198-224
SEQ ID NO. 396: AA199-225
SEQ ID NO. 397: AA200-226
SEQ ID NO. 398: AA201-227
SEQ ID NO. 399: AA202-228
SEQ ID NO. 400: AA203-229
SEQ ID NO. 401: AA204-230
SEQ ID NO. 402: AA205-231
SEQ ID NO. 403: AA206-232
SEQ ID NO. 404: AA207-233
SEQ ID NO. 405: AA208-234
SEQ ID NO. 406: AA209-235
SEQ ID NO. 407: AA210-236
SEQ ID NO. 408: AA211-237
SEQ ID NO. 409: AA212-238
SEQ ID NO. 410: AA213-239
SEQ ID NO. 411: AA214-240
SEQ ID NO. 412: AA215-241
SEQ ID NO. 413: AA216-242
SEQ ID NO. 414: AA190-215
SEQ ID NO. 415: AA191-216
SEQ ID NO. 416: AA192-217
SEQ ID NO. 417: AA193-218
SEQ ID NO. 418: AA194-219
SEQ ID NO. 419: AA195-220
SEQ ID NO. 420: AA196-221
SEQ ID NO. 421: AA197-222
SEQ ID NO. 422: AA198-223
SEQ ID NO. 423: AA199-224
SEQ ID NO. 424: AA200-225
SEQ ID NO. 425: AA201-226
SEQ ID NO. 426: AA202-227
SEQ ID NO. 427: AA203-228
SEQ ID NO. 428: AA204-229
SEQ ID NO. 429: AA205-230
SEQ ID NO. 430: AA206-231
SEQ ID NO. 431: AA207-232
SEQ ID NO. 432: AA208-233
SEQ ID NO. 433: AA209-234
SEQ ID NO. 434: AA210-235
SEQ ID NO. 435: AA211-236
SEQ ID NO. 436: AA212-237
SEQ ID NO. 437: AA213-238
SEQ ID NO. 438: AA214-239
SEQ ID NO. 439: AA215-240
SEQ ID NO. 440: AA216-241
SEQ ID NO. 441: AA217-242
SEQ ID NO. 442: AA218-243
SEQ ID NO. 443: AA190-214
SEQ ID NO. 444: AA191-215
SEQ ID NO. 445: AA192-216
SEQ ID NO. 446: AA193-217
SEQ ID NO. 447: AA194-218
SEQ ID NO. 448: AA195-219
SEQ ID NO. 449: AA196-220
SEQ ID NO. 450: AA197-221
SEQ ID NO. 451: AA198-222
SEQ ID NO. 452: AA199-223
SEQ ID NO. 453: AA200-224
SEQ ID NO. 454: AA201-225
SEQ ID NO. 455: AA202-226
SEQ ID NO. 456: AA203-227
SEQ ID NO. 457: AA204-228
SEQ ID NO. 458: AA205-229
SEQ ID NO. 459: AA206-230
SEQ ID NO. 460: AA207-231
SEQ ID NO. 461: AA208-232
SEQ ID NO. 462: AA209-233
SEQ ID NO. 463: AA210-234
SEQ ID NO. 464: AA211-235
SEQ ID NO. 465: AA212-236
SEQ ID NO. 466: AA213-237
SEQ ID NO. 467: AA214-238
SEQ ID NO. 468: AA215-239
SEQ ID NO. 469: AA216-240
SEQ ID NO. 470: AA217-241
SEQ ID NO. 471: AA218-242
SEQ ID NO. 472: AA219-243
SEQ ID NO. 473: AA190-213
SEQ ID NO. 474: AA191-214
SEQ ID NO. 475: AA192-215
SEQ ID NO. 476: AA193-216
SEQ ID NO. 477: AA194-217
SEQ ID NO. 478: AA195-218
SEQ ID NO. 479: AA196-219
SEQ ID NO. 480: AA197-220
SEQ ID NO. 481: AA198-221
SEQ ID NO. 482: AA199-222
SEQ ID NO. 483: AA200-223
SEQ ID NO. 484: AA201-224
SEQ ID NO. 485: AA202-225
SEQ ID NO. 486: AA203-226
SEQ ID NO. 487: AA204-227
SEQ ID NO. 488: AA205-228
SEQ ID NO. 489: AA206-229
SEQ ID NO. 490: AA207-230
SEQ ID NO. 491: AA208-231
SEQ ID NO. 492: AA209-232
SEQ ID NO. 493: AA210-233
SEQ ID NO. 494: AA211-234
SEQ ID NO. 495: AA212-235
SEQ ID NO. 496: AA213-236
SEQ ID NO. 497: AA214-237
SEQ ID NO. 498: AA215-238
SEQ ID NO. 499: AA216-239
SEQ ID NO. 500: AA217-240
SEQ ID NO. 501: AA218-241
SEQ ID NO. 502: AA219-242
SEQ ID NO. 503: AA220-243
SEQ ID NO. 504: AA190-212
SEQ ID NO. 505: AA191-213
SEQ ID NO. 506: AA192-214
SEQ ID NO. 507: AA193-215
SEQ ID NO. 508: AA194-216
SEQ ID NO. 509: AA195-217
SEQ ID NO. 510: AA196-218
SEQ ID NO. 511: AA197-219
SEQ ID NO. 512: AA198-220
SEQ ID NO. 513: AA199-221
SEQ ID NO. 514: AA200-222
SEQ ID NO. 515: AA201-223
SEQ ID NO. 516: AA202-224
SEQ ID NO. 517: AA203-225
SEQ ID NO. 518: AA204-226
SEQ ID NO. 519: AA205-227
SEQ ID NO. 520: AA206-228
SEQ ID NO. 521: AA207-229
SEQ ID NO. 522: AA208-230
SEQ ID NO. 523: AA209-231
SEQ ID NO. 524: AA210-232
SEQ ID NO. 525: AA211-233
SEQ ID NO. 526: AA212-234
SEQ ID NO. 527: AA213-235
SEQ ID NO. 528: AA214-236
SEQ ID NO. 529: AA215-237
SEQ ID NO. 530: AA216-238
SEQ ID NO. 531: AA217-239
SEQ ID NO. 532: AA218-240
SEQ ID NO. 533: AA219-241
SEQ ID NO. 534: AA220-242
SEQ ID NO. 535: AA221-243
SEQ ID NO. 536: AA190-211
SEQ ID NO. 537: AA191-212
SEQ ID NO. 538: AA192-213
SEQ ID NO. 539: AA193-214
SEQ ID NO. 540: AA194-215
SEQ ID NO. 541: AA195-216
SEQ ID NO. 542: AA196-217
SEQ ID NO. 543: AA197-218
SEQ ID NO. 544: AA198-219
SEQ ID NO. 545: AA199-220
SEQ ID NO. 546: AA200-221
SEQ ID NO. 547: AA201-222
SEQ ID NO. 548: AA202-223
SEQ ID NO. 549: AA203-224
SEQ ID NO. 550: AA204-225
SEQ ID NO. 551: AA205-226
SEQ ID NO. 552: AA206-227
SEQ ID NO. 553: AA207-228
SEQ ID NO. 554: AA208-229
SEQ ID NO. 555: AA209-230
SEQ ID NO. 556: AA210-231
SEQ ID NO. 557: AA211-232
SEQ ID NO. 558: AA212-233
SEQ ID NO. 559: AA213-234
SEQ ID NO. 560: AA214-235
SEQ ID NO. 561: AA215-236
SEQ ID NO. 562: AA216-237
SEQ ID NO. 563: AA217-238
SEQ ID NO. 564: AA218-239
SEQ ID NO. 565: AA219-240
SEQ ID NO. 566: AA220-241
SEQ ID NO. 567: AA221-242
SEQ ID NO. 568: AA222-243
SEQ ID NO. 569: AA190-210
SEQ ID NO. 570: AA191-211
SEQ ID NO. 571: AA192-212
SEQ ID NO. 572: AA193-213
SEQ ID NO. 573: AA194-214
SEQ ID NO. 574: AA195-215
SEQ ID NO. 575: AA196-216
SEQ ID NO. 576: AA197-217
SEQ ID NO. 577: AA198-218
SEQ ID NO. 578: AA199-219
SEQ ID NO. 579: AA200-220
SEQ ID NO. 580: AA201-221
SEQ ID NO. 581: AA202-222
SEQ ID NO. 582: AA203-223
SEQ ID NO. 583: AA204-224
SEQ ID NO. 584: AA205-225
SEQ ID NO. 585: AA206-226
SEQ ID NO. 586: AA207-227
SEQ ID NO. 587: AA208-228
SEQ ID NO. 588: AA209-229
SEQ ID NO. 589: AA210-230
SEQ ID NO. 590: AA211-231
SEQ ID NO. 591: AA212-232
SEQ ID NO. 592: AA213-233
SEQ ID NO. 593: AA214-234
SEQ ID NO. 594: AA215-235
SEQ ID NO. 595: AA216-236
SEQ ID NO. 596: AA217-237
SEQ ID NO. 597: AA218-238
SEQ ID NO. 598: AA219-239
SEQ ID NO. 599: AA220-240
SEQ ID NO. 600: AA221-241
SEQ ID NO. 601: AA222-242
SEQ ID NO. 602: AA223-243
SEQ ID NO. 603: AA190-209
SEQ ID NO. 604: AA191-210
SEQ ID NO. 605: AA192-211
SEQ ID NO. 606: AA193-212
SEQ ID NO. 607: AA194-213
SEQ ID NO. 608: AA195-214
SEQ ID NO. 609: AA196-215
SEQ ID NO. 610: AA197-216
SEQ ID NO. 611: AA198-217
SEQ ID NO. 612: AA199-218
SEQ ID NO. 613: AA200-219
SEQ ID NO. 614: AA201-220
SEQ ID NO. 615: AA202-221
SEQ ID NO. 616: AA203-222
SEQ ID NO. 617: AA204-223
SEQ ID NO. 618: AA205-224
SEQ ID NO. 619: AA206-225
SEQ ID NO. 620: AA207-226
SEQ ID NO. 621: AA208-227
SEQ ID NO. 622: AA209-228
SEQ ID NO. 623: AA210-229
SEQ ID NO. 624: AA211-230
SEQ ID NO. 625: AA212-231
SEQ ID NO. 626: AA213-232
SEQ ID NO. 627: AA214-233
SEQ ID NO. 628: AA215-234
SEQ ID NO. 629: AA216-235
SEQ ID NO. 630: AA217-236
SEQ ID NO. 631: AA218-237
SEQ ID NO. 632: AA219-238
SEQ ID NO. 633: AA220-239
SEQ ID NO. 634: AA221-240
SEQ ID NO. 635: AA222-241
SEQ ID NO. 636: AA223-242
SEQ ID NO. 637: AA224-243
SEQ ID NO. 638: AA190-208
SEQ ID NO. 639: AA191-209
SEQ ID NO. 640: AA192-210
SEQ ID NO. 641: AA193-211
SEQ ID NO. 642: AA194-212
SEQ ID NO. 643: AA195-213
SEQ ID NO. 644: AA196-214
SEQ ID NO. 645: AA197-215
SEQ ID NO. 646: AA198-216
SEQ ID NO. 647: AA199-217
SEQ ID NO. 648: AA200-218
SEQ ID NO. 649: AA201-219
SEQ ID NO. 650: AA202-220
SEQ ID NO. 651: AA203-221
SEQ ID NO. 652: AA204-222
SEQ ID NO. 653: AA205-223
SEQ ID NO. 654: AA206-224
SEQ ID NO. 655: AA207-225
SEQ ID NO. 656: AA208-226
SEQ ID NO. 657: AA209-227
SEQ ID NO. 658: AA210-228
SEQ ID NO. 659: AA211-229
SEQ ID NO. 660: AA212-230
SEQ ID NO. 661: AA213-231
SEQ ID NO. 662: AA214-232
SEQ ID NO. 663: AA215-233
SEQ ID NO. 664: AA216-234
SEQ ID NO. 665: AA217-235
SEQ ID NO. 666: AA218-236
SEQ ID NO. 667: AA219-237
SEQ ID NO. 668: AA220-238
SEQ ID NO. 669: AA221-239
SEQ ID NO. 670: AA222-240
SEQ ID NO. 671: AA223-241
SEQ ID NO. 672: AA224-242
SEQ ID NO. 673: AA225-243
SEQ ID NO. 674: AA233-242
SEQ ID NO. 675: AA191-208
SEQ ID NO. 676: AA192-209
SEQ ID NO. 677: AA193-210
SEQ ID NO. 678: AA194-211
SEQ ID NO. 679: AA195-212
SEQ ID NO. 680: AA196-213
SEQ ID NO. 681: AA197-214
SEQ ID NO. 682: AA198-215
SEQ ID NO. 683: AA200-217
SEQ ID NO. 684: AA201-218
SEQ ID NO. 685: AA202-219
SEQ ID NO. 686: AA203-220
SEQ ID NO. 687: AA204-221
SEQ ID NO. 688: AA205-222
SEQ ID NO. 689: AA206-223
SEQ ID NO. 690: AA207-224
SEQ ID NO. 691: AA209-226
SEQ ID NO. 692: AA210-227
SEQ ID NO. 693: AA211-228
SEQ ID NO. 694: AA212-229
SEQ ID NO. 695: AA213-230
SEQ ID NO. 696: AA214-231
SEQ ID NO. 697: AA215-232
SEQ ID NO. 698: AA216-233
SEQ ID NO. 699: AA218-235
SEQ ID NO. 700: AA219-236
SEQ ID NO. 701: AA220-237
SEQ ID NO. 702: AA221-238
SEQ ID NO. 703: AA222-239
SEQ ID NO. 704: AA223-240
SEQ ID NO. 705: AA224-241
SEQ ID NO. 706: AA225-242
SEQ ID NO. 707: AA190-206
SEQ ID NO. 708: AA191-207
SEQ ID NO. 709: AA192-208
SEQ ID NO. 710: AA193-209
SEQ ID NO. 711: AA194-210
SEQ ID NO. 712: AA195-211
SEQ ID NO. 713: AA196-212
SEQ ID NO. 714: AA197-213
SEQ ID NO. 715: AA198-214
SEQ ID NO. 716: AA199-215
SEQ ID NO. 717: AA200-216
SEQ ID NO. 718: AA201-217
SEQ ID NO. 719: AA202-218
SEQ ID NO. 720: AA203-219
SEQ ID NO. 721: AA204-220
SEQ ID NO. 722: AA205-221
SEQ ID NO. 723: AA206-222
SEQ ID NO. 724: AA207-223
SEQ ID NO. 725: AA208-224
SEQ ID NO. 726: AA209-225
SEQ ID NO. 727: AA210-226
SEQ ID NO. 728: AA211-227
SEQ ID NO. 729: AA212-228
SEQ ID NO. 730: AA213-229
SEQ ID NO. 731: AA214-230
SEQ ID NO. 732: AA215-231
SEQ ID NO. 733: AA216-232
SEQ ID NO. 734: AA217-233
SEQ ID NO. 735: AA218-234
SEQ ID NO. 736: AA219-235
SEQ ID NO. 737: AA220-236
SEQ ID NO. 738: AA221-237
SEQ ID NO. 739: AA222-238
SEQ ID NO. 740: AA223-239
SEQ ID NO. 741: AA224-240
SEQ ID NO. 742: AA225-241
SEQ ID NO. 743: AA226-242
SEQ ID NO. 744: AA227-243
SEQ ID NO. 745: AA190-205
SEQ ID NO. 746: AA191-206
SEQ ID NO. 747: AA192-207
SEQ ID NO. 748: AA193-208
SEQ ID NO. 749: AA194-209
SEQ ID NO. 750: AA195-210
SEQ ID NO. 751: AA196-211
SEQ ID NO. 752: AA197-212
SEQ ID NO. 753: AA198-213
SEQ ID NO. 754: AA199-214
SEQ ID NO. 755: AA200-215
SEQ ID NO. 756: AA201-216
SEQ ID NO. 757: AA202-217
SEQ ID NO. 758: AA203-218
SEQ ID NO. 759: AA204-219
SEQ ID NO. 760: AA205-220
SEQ ID NO. 761: AA206-221
SEQ ID NO. 762: AA207-222
SEQ ID NO. 763: AA208-223
SEQ ID NO. 764: AA209-224
SEQ ID NO. 765: AA210-225
SEQ ID NO. 766: AA211-226
SEQ ID NO. 767: AA212-227
SEQ ID NO. 768: AA213-228
SEQ ID NO. 769: AA214-229
SEQ ID NO. 770: AA215-230
SEQ ID NO. 771: AA216-231
SEQ ID NO. 772: AA217-232
SEQ ID NO. 773: AA218-233
SEQ ID NO. 774: AA219-234
SEQ ID NO. 775: AA220-235
SEQ ID NO. 776: AA221-236
SEQ ID NO. 777: AA222-237
SEQ ID NO. 778: AA223-238
SEQ ID NO. 779: AA224-239
SEQ ID NO. 780: AA225-240
SEQ ID NO. 781: AA226-241
SEQ ID NO. 782: AA227-242
SEQ ID NO. 783: AA228-243
SEQ ID NO. 784: AA190-204
SEQ ID NO. 785: AA191-205
SEQ ID NO. 786: AA192-206
SEQ ID NO. 787: AA193-207
SEQ ID NO. 788: AA194-208
SEQ ID NO. 789: AA195-209
SEQ ID NO. 790: AA196-210
SEQ ID NO. 791: AA197-211
SEQ ID NO. 792: AA198-212
SEQ ID NO. 793: AA199-213
SEQ ID NO. 794: AA200-214
SEQ ID NO. 795: AA201-215
SEQ ID NO. 796: AA202-216
SEQ ID NO. 797: AA203-217
SEQ ID NO. 798: AA204-218
SEQ ID NO. 799: AA205-219
SEQ ID NO. 800: AA206-220
SEQ ID NO. 801: AA207-221
SEQ ID NO. 802: AA208-222
SEQ ID NO. 803: AA209-223
SEQ ID NO. 804: AA210-224
SEQ ID NO. 805: AA211-225
SEQ ID NO. 806: AA212-226
SEQ ID NO. 807: AA213-227
SEQ ID NO. 808: AA214-228
SEQ ID NO. 809: AA215-229
SEQ ID NO. 810: AA216-230
SEQ ID NO. 811: AA217-231
SEQ ID NO. 812: AA218-232
SEQ ID NO. 813: AA219-233
SEQ ID NO. 814: AA220-234
SEQ ID NO. 815: AA221-235
SEQ ID NO. 816: AA222-236
SEQ ID NO. 817: AA223-237
SEQ ID NO. 818: AA224-238
SEQ ID NO. 819: AA225-239
SEQ ID NO. 820: AA226-240
SEQ ID NO. 821: AA227-241
SEQ ID NO. 822: AA228-242
SEQ ID NO. 823: AA229-243
SEQ ID NO. 824: AA190-203
SEQ ID NO. 825: AA191-204
SEQ ID NO. 826: AA192-205
SEQ ID NO. 827: AA193-206
SEQ ID NO. 828: AA194-207
SEQ ID NO. 829: AA195-208
SEQ ID NO. 830: AA196-209
SEQ ID NO. 831: AA197-210
SEQ ID NO. 832: AA198-211
SEQ ID NO. 833: AA199-212
SEQ ID NO. 834: AA200-213
SEQ ID NO. 835: AA201-214
SEQ ID NO. 836: AA202-215
SEQ ID NO. 837: AA203-216
SEQ ID NO. 838: AA204-217
SEQ ID NO. 839: AA205-218
SEQ ID NO. 840: AA206-219
SEQ ID NO. 841: AA207-220
SEQ ID NO. 842: AA208-221
SEQ ID NO. 843: AA209-222
SEQ ID NO. 844: AA210-223
SEQ ID NO. 845: AA211-224
SEQ ID NO. 846: AA212-225
SEQ ID NO. 847: AA213-226
SEQ ID NO. 848: AA214-227
SEQ ID NO. 849: AA215-228
SEQ ID NO. 850: AA216-229
SEQ ID NO. 851: AA217-230
SEQ ID NO. 852: AA218-231
SEQ ID NO. 853: AA219-232
SEQ ID NO. 854: AA220-233
SEQ ID NO. 855: AA221-234
SEQ ID NO. 856: AA222-235
SEQ ID NO. 857: AA223-236
SEQ ID NO. 858: AA224-237
SEQ ID NO. 859: AA225-238
SEQ ID NO. 860: AA226-239
SEQ ID NO. 861: AA227-240
SEQ ID NO. 862: AA228-241
SEQ ID NO. 863: AA229-242
SEQ ID NO. 864: AA230-243
SEQ ID NO. 865: AA190-202
SEQ ID NO. 866: AA191-203
SEQ ID NO. 867: AA192-204
SEQ ID NO. 868: AA193-205
SEQ ID NO. 869: AA194-206
SEQ ID NO. 870: AA195-207
SEQ ID NO. 871: AA196-208
SEQ ID NO. 872: AA197-209
SEQ ID NO. 873: AA198-210
SEQ ID NO. 874: AA199-211
SEQ ID NO. 875: AA200-212
SEQ ID NO. 876: AA201-213
SEQ ID NO. 877: AA202-214
SEQ ID NO. 878: AA203-215
SEQ ID NO. 879: AA204-216
SEQ ID NO. 880: AA205-217
SEQ ID NO. 881: AA206-218
SEQ ID NO. 882: AA207-219
SEQ ID NO. 883: AA208-220
SEQ ID NO. 884: AA209-221
SEQ ID NO. 885: AA210-222
SEQ ID NO. 886: AA211-223
SEQ ID NO. 887: AA212-224
SEQ ID NO. 888: AA213-225
SEQ ID NO. 889: AA214-226
SEQ ID NO. 890: AA215-227
SEQ ID NO. 891: AA216-228
SEQ ID NO. 892: AA217-229
SEQ ID NO. 893: AA218-230
SEQ ID NO. 894: AA219-231
SEQ ID NO. 895: AA220-232
SEQ ID NO. 896: AA221-233
SEQ ID NO. 897: AA222-234
SEQ ID NO. 898: AA223-235
SEQ ID NO. 899: AA224-236
SEQ ID NO. 900: AA225-237
SEQ ID NO. 901: AA226-238
SEQ ID NO. 902: AA227-239
SEQ ID NO. 903: AA228-240
SEQ ID NO. 904: AA229-241
SEQ ID NO. 905: AA230-242
SEQ ID NO. 906: AA231-243
SEQ ID NO. 907: AA190-201
SEQ ID NO. 908: AA191-202
SEQ ID NO. 909: AA192-203
SEQ ID NO. 910: AA193-204
SEQ ID NO. 911: AA194-205
SEQ ID NO. 912: AA195-206
SEQ ID NO. 913: AA196-207
SEQ ID NO. 914: AA197-208
SEQ ID NO. 915: AA198-209
SEQ ID NO. 916: AA199-210
SEQ ID NO. 917: AA200-211
SEQ ID NO. 918: AA201-212
SEQ ID NO. 919: AA202-213
SEQ ID NO. 920: AA203-214
SEQ ID NO. 921: AA204-215
SEQ ID NO. 922: AA205-216
SEQ ID NO. 923: AA206-217
SEQ ID NO. 924: AA207-218
SEQ ID NO. 925: AA208-219
SEQ ID NO. 926: AA209-220
SEQ ID NO. 927: AA210-221
SEQ ID NO. 928: AA211-222
SEQ ID NO. 929: AA212-223
SEQ ID NO. 930: AA213-224
SEQ ID NO. 931: AA214-225
SEQ ID NO. 932: AA215-226
SEQ ID NO. 933: AA216-227
SEQ ID NO. 934: AA217-228
SEQ ID NO. 935: AA218-229
SEQ ID NO. 936: AA219-230
SEQ ID NO. 937: AA220-231
SEQ ID NO. 938: AA221-232
SEQ ID NO. 939: AA222-233
SEQ ID NO. 940: AA223-234
SEQ ID NO. 941: AA224-235
SEQ ID NO. 942: AA225-236
SEQ ID NO. 943: AA226-237
SEQ ID NO. 944: AA227-238
SEQ ID NO. 945: AA228-239
SEQ ID NO. 946: AA229-240
SEQ ID NO. 947: AA230-241
SEQ ID NO. 948: AA231-242
SEQ ID NO. 949: AA232-243
SEQ ID NO. 950: AA190-200
SEQ ID NO. 951: AA191-201
SEQ ID NO. 952: AA192-202
SEQ ID NO. 953: AA193-203
SEQ ID NO. 954: AA194-204
SEQ ID NO. 955: AA195-205
SEQ ID NO. 956: AA196-206
SEQ ID NO. 957: AA197-207
SEQ ID NO. 958: AA198-208
SEQ ID NO. 959: AA199-209
SEQ ID NO. 960: AA200-210
SEQ ID NO. 961: AA201-211
SEQ ID NO. 962: AA202-212
SEQ ID NO. 963: AA203-213
SEQ ID NO. 964: AA204-214
SEQ ID NO. 965: AA205-215
SEQ ID NO. 966: AA206-216
SEQ ID NO. 967: AA207-217
SEQ ID NO. 968: AA208-218
SEQ ID NO. 969: AA209-219
SEQ ID NO. 970: AA210-220
SEQ ID NO. 971: AA211-221
SEQ ID NO. 972: AA212-222
SEQ ID NO. 973: AA213-223
SEQ ID NO. 974: AA214-224
SEQ ID NO. 975: AA215-225
SEQ ID NO. 976: AA216-226
SEQ ID NO. 977: AA217-227
SEQ ID NO. 978: AA218-228
SEQ ID NO. 979: AA219-229
SEQ ID NO. 980: AA220-230
SEQ ID NO. 981: AA221-231
SEQ ID NO. 982: AA222-232
SEQ ID NO. 983: AA223-233
SEQ ID NO. 984: AA224-234
SEQ ID NO. 985: AA225-235
SEQ ID NO. 986: AA226-236
SEQ ID NO. 987: AA227-237
SEQ ID NO. 988: AA228-238
SEQ ID NO. 989: AA229-239
SEQ ID NO. 990: AA230-240
SEQ ID NO. 991: AA231-241
SEQ ID NO. 992: AA232-242
SEQ ID NO. 993: AA233-243
SEQ ID NO. 994: AA190-199
SEQ ID NO. 995: AA191-200
SEQ ID NO. 996: AA192-201
SEQ ID NO. 997: AA193-202
SEQ ID NO. 998: AA194-203
SEQ ID NO. 999: AA195-204
SEQ ID NO. 1000: AA196-205
SEQ ID NO. 1001: AA197-206
SEQ ID NO. 1002: AA198-207
SEQ ID NO. 1003: AA199-208
SEQ ID NO. 1004: AA200-209
SEQ ID NO. 1005: AA201-210
SEQ ID NO. 1006: AA202-211
SEQ ID NO. 1007: AA203-212
SEQ ID NO. 1008: AA204-213
SEQ ID NO. 1009: AA205-214
SEQ ID NO. 1010: AA206-215
SEQ ID NO. 1011: AA207-216
SEQ ID NO. 1012: AA208-217
SEQ ID NO. 1013: AA209-218
SEQ ID NO. 1014: AA210-219
SEQ ID NO. 1015: AA211-220
SEQ ID NO. 1016: AA212-221
SEQ ID NO. 1017: AA213-222
SEQ ID NO. 1018: AA214-223
SEQ ID NO. 1019: AA215-224
SEQ ID NO. 1020: AA216-225
SEQ ID NO. 1021: AA217-226
SEQ ID NO. 1022: AA218-227
SEQ ID NO. 1023: AA219-228
SEQ ID NO. 1024: AA220-229
SEQ ID NO. 1025: AA221-230
SEQ ID NO. 1026: AA222-231
SEQ ID NO. 1027: AA223-232
SEQ ID NO. 1028: AA224-233
SEQ ID NO. 1029: AA225-234
SEQ ID NO. 1030: AA226-235
SEQ ID NO. 1031: AA227-236
SEQ ID NO. 1032: AA228-237
SEQ ID NO. 1033: AA229-238
SEQ ID NO. 1034: AA230-239
SEQ ID NO. 1035: AA231-240

### EXAMPLES

### Example 1

### Production of Polypeptides

A person skilled in the art would appreciate that polypeptides according to the present invention having an amino acid sequence of SEQ ID NO: 1 to 1035 could be produced in using methods known in the art, for example by chemical synthesis of polypeptides or heterologous expression systems such as for example using a method as described below for the polypeptides consisting of amino acids 1-243, and 1-189, 44-189, 44-243, and 190-243 of the full-length human apoA-I protein. Note that the polypeptide fragment consisting of amino acids 190-243 of the full-length human apoA-I protein has the sequence of SEQ ID NO:1.

Expression and Purification of Recombinant Human apoA-I Variants

Human apoA-I variants (full-length and truncated variants, produced by site directed mutagenesis, corresponding to amino acids 1-243, and 1-189, 44-189, 44-243, and 190-243, respectively) were expressed in Escherichia coli strain BL21 Star (DE3)pLysS cells (Invitrogen) from the human apoA-I gene containing a hexa-His affinity tag at the N-terminus (Lagerstedt et al. (2007) J Biol Chem 282: 9143-9149). Briefly, the gene (full-length or truncated variants of the gene) was cloned into the pEXP-5 plasmid (Novagen, Inc, Madison) and transferred into the bacteria and cultivated at 37° C. in LB medium with 50 μg/ml of ampicillin and 34 μg/ml of chloramphenicol. Protein expression was induced for 3-4 hours following the addition of 0.5 mmol/l isopropyl-beta-thiogalactopyranoside (Sigma). Following cell disruption, apoA-I was purified from the soluble fraction of the cells using a His-Trap-Nickel-chelating column (GE Healthcare) and a mobile phase of phosphate-buffered saline (PBS), pH 7.4 with 3 mol/l guanidine. The protein was then washed in PBS (pH 7.4) containing 100 mmol/l imidazole, and then eluted with PBS containing 500 mmol/l imidazole. Imidazole was removed from the protein sample by using desalting columns (GE Healthcare) equilibrated with PBS, pH 7.4. Protein purity was analyzed by SDS-PAGE and concentration was determined by the BCA method (Pierce) or using a nanodrop 2000c spectrophotometer (Thermo scientific).

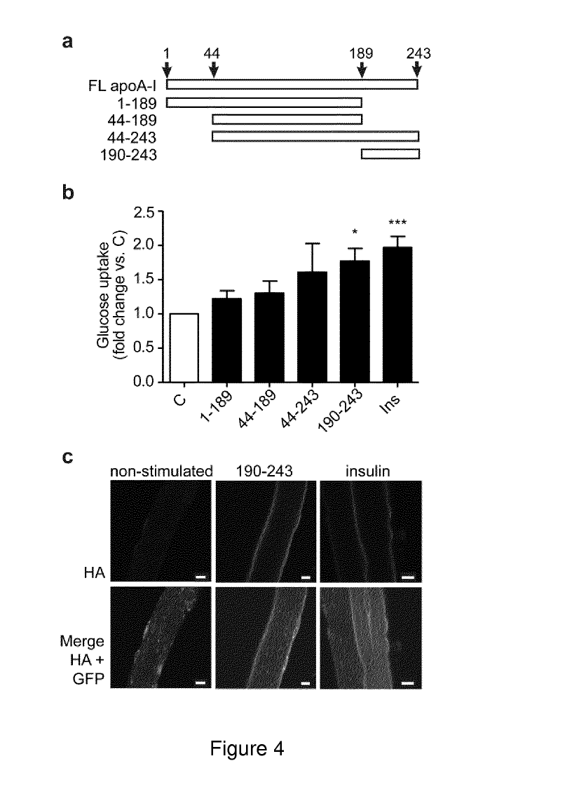

The above method produced full-length apoA-I polypeptide and truncated variants (polypeptide fragments of full-length apoA-I) consisting of amino acids 1-243, 1-189, 44-189, 44-243, and 190-243 of human full-length apoA-I. FIG. 4a shows a schematic overview of the truncated variants.

### Example 2

### Testing Induced Glucose Uptake in Myoblast Cells

Full length human lipid-free apoA-I and mature spherical high density lipoprotein (HDL) comprising the full length human apoA-I is useful for studies of induced glucose uptake. In order to investigate the effect of synthetic reconstituted discodial HDL (rHDL) on GLUT4 translocation and glucose uptake, recombinant human apoA-I and reconstituted HDL needed for cell incubations were produced and tested for their effects on induced glucose uptake in L6 myoblasts. The relative effect of specific regions of apoA-I to increase glucose uptake was also investigated using truncated protein fragments corresponding to residues 1-189 (N-terminal/central domain), 44-189 (central domain), 44-243 (central/C-terminal domain) and 190-243 (C-terminal domain) of full-length apoA-I (FIG. 4a). The latter protein fragment corresponds to a polypeptide having the amino acid sequence of SEQ ID NO:1.

### Cell Culture

L6 myoblasts (ATCC #CRL-1458) were grown in α-MEM (Invitrogen) supplemented with 10% FBS (Sigma) and 1% antibiotic/antimycotic (Penicillin, streptomycin, amphotericin B; Invitrogen). Differentiation to myotubes was achieved by switching from growth media to 2% FBS a-MEM for 6-7 or 6-12 days. Cells were maintained at 37° C. and 5% CO₂.

### Glucose Uptake Measurements

Prior to stimulation (or treatment) cells were starved for 2 hours in serum-free a-MEM and all subsequent treatments, which were performed in triplicate and included cytochalasin B (Sigma) as a measure of cell associated non-specific radioactivity, were performed in uptake buffer (140 mmol/l NaCl, 20 mmol/l HEPES, 5 mmol/l KCl, 2.5 mmol/l MgSO₄, 1 mmol/l CaCl₂, pH 7.4). After stimulation, treatments were replaced with 10 μmol/l 2-deoxy-D-glucose (Sigma), 10 μmol/l 2-[3H]-deoxy-D-glucose (Perkin Elmer) in uptake buffer for 15 mins at room temperature. Cells were then washed twice with ice cold PBS and lysed with 1 mol/l NaOH on ice. Lysates were collected and radioactivity quantified by scintillation counting.

### Statistical Analysis

All data are displayed as mean±SEM unless indicated otherwise. Where appropriate, analysis was performed by two-tailed student's t test or one-way ANOVA with Bonferroni's post hoc test using Microsoft Excel and Graph Pad Prism software. p=≦0.05 was considered significant.

Production of Reconstituted HDL (rHDL)

1-palmitoyl-2-oleoyl-sn-glycero-3-phosphocholine (POPC) or 1,2-dimyristoyl-sn-glycero-3-phosphocholine (DMPC) (Avanti Polar Lipids) were dissolved in chloroform:methanol (3:1) which was evaporated under a stream of nitrogen gas and the resulting lipid film was resuspended in PBS. For POPC rHDL, deoxycholate was added to the POPC emulsion at a 2:1 molar ratio (deoxycholate:POPC) and incubated with apoA-I at a 100:1 molar ratio (phospholipid:protein) for 1 hour at 22° C. (Lagerstedt et al. (2011) Journal of Biological Chemistry, 2011; 286:2966-2975). Deoxycholate was removed from the POPC rHDL preparation by extensive dialysis against PBS. DMPC rHDL were prepared according to Petrlova et al. (2012) J Lipid Res 53: 390-398. Briefly, the DMPC emulsion was passed through a polycarbonate membrane with 100 nm pore size using the LiposoFast system (Avestin) a minimum of 20-times. The resulting vesicles were incubated with apoA-I at a 100:1 molar ratio (phospholipid:protein) for 4 days at 22° C. ApoA-I dimers, indicative of rHDL formation, was confirmed by blue native PAGE (Invitrogen). Treatments were performed with POPC rHDL unless otherwise indicated.

### Western Blotting

Prior to stimulation or treatment cultured myoblast cells were serum starved for 4 hours in serum-free α-MEM and all subsequent treatments, including insulin or phenformin as positive controls (Sigma), were performed in serum free a-MEM. After treatments, cells were washed with ice cold PBS buffer and lysed on ice using a non-denaturing lysis buffer (1% Triton X-100, 50 mmol/l Tris, 150 mmol/l NaCl, pH 8.0) containing protease and phosphatase inhibitors (Roche). Lysates were centrifuged at 16,000×g, 20 minutes at 4° C. and BCA protein assay (Pierce) was performed on supernatants. Equal protein amounts were separated by SDS-PAGE and transferred to nitrocellulose membranes. pAMPK, AMPK, pACC, pAkt, Akt and tubulin were used for immunodetection with IRDye 800CW and 680RD secondary antibodies (LI-COR). Blots were imaged using the Odyssey Fc system and quantified using Image studio v2.0 software.

rHDL Induces Glucose Uptake and GLUT4 Translocation in Skeletal Muscle Cells

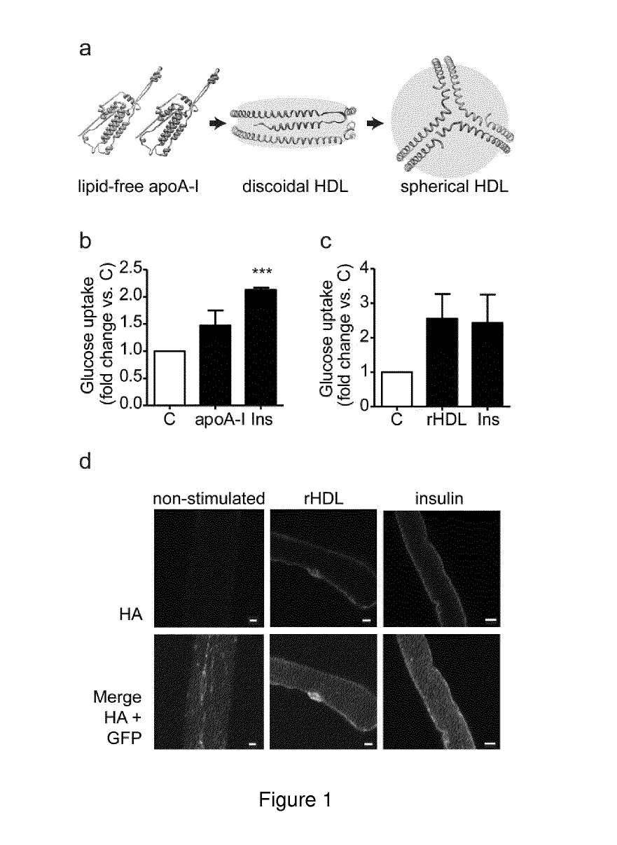

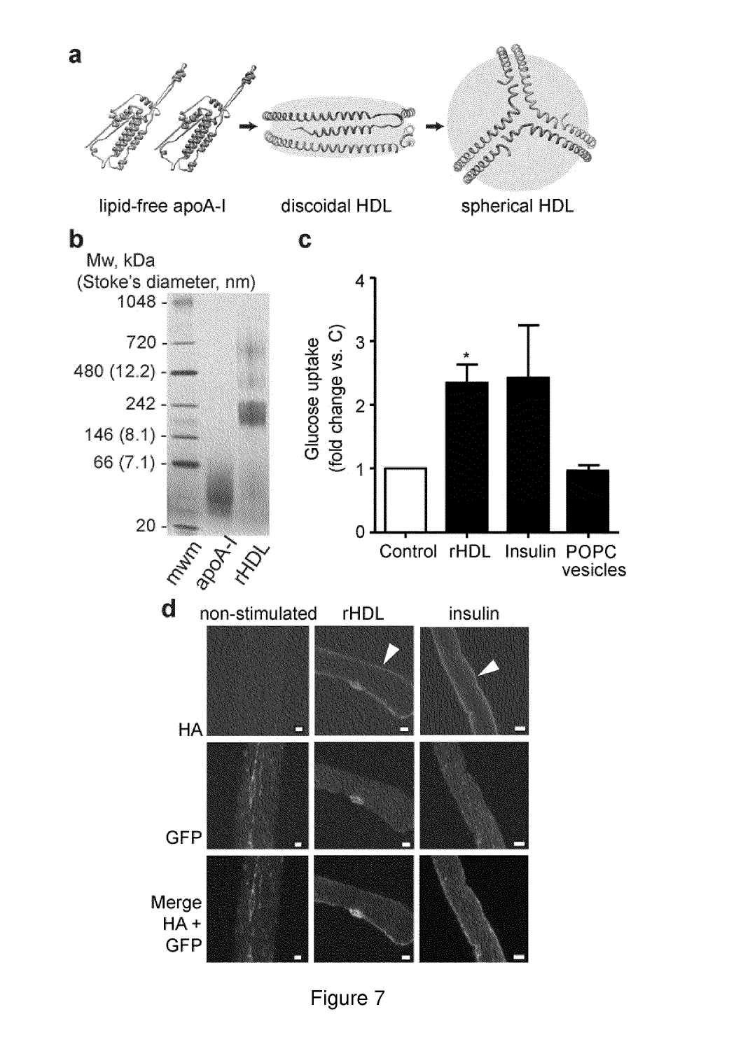

In order to investigate the effect of discodial HDL (rHDL) on GLUT4 translocation and glucose uptake, we produced recombinant human apoA-I and reconstituted HDL needed for cell incubations. L6 myotubes were incubated with 50 μg/ml (1.6 μmol/l) apoA-I for 1 hour followed by a glucose uptake assay as described above. Treatment by incubation with insulin increased the glucose uptake by 2-fold, and the apoA-I treated cells displayed an increased glucose uptake of 1.3-fold (FIG. 1b). Next, L6 myotubes were treated by incubation with 50 μg/ml discodial rHDL (expressed as total concentration of apoA-I in rHDL, given 2 apoA-I molecules per particle) for 1 hour. The rHDL treatment induced a glucose uptake that was 2.6-fold (p=0.095) over basal, which was similar to insulin stimulation (2.4±1.0-fold). However, this did not reach significance due to a 4-fold effect for both apoA-I and insulin treatments in one experiment compared to effects in the 1.6-1.9-fold range in other experiments (FIG. 1c). This experiment was thereafter repeated in more controlled manner including cell number and quality (cells were similarly treated during differentiation from myoblasts to multinuclear myotubes, with respect to the time between splitting cells, cell density, passage of cells used.) (FIG. 7c). A similar increase in glucose uptake (about 2.5 fold over Control) was then observed that also showed statistical significance.

The relative effect of specific regions of apoA-I to increase glucose uptake was investigated using truncated protein fragments corresponding to residues 1-189 (N-terminal/central domain), 44-189 (central domain), 44-243 (central/C-terminal domain) and 190-243 (C-terminal domain) of full-length apoA-I (FIG. 4a). The treatments with incubation of truncated protein fragments were performed as described above for treatment of L6 myotubes with full-length apoA-I and followed by a glucose uptake assay as described above.

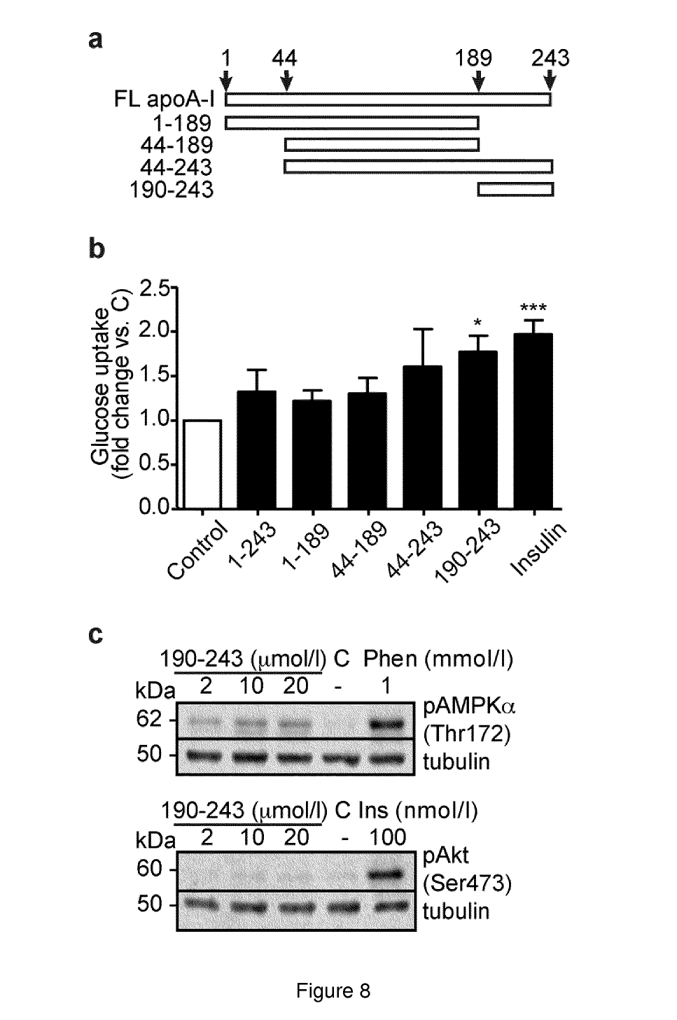

As can be seen in FIGS. 4b and 8b, all four peptides induced glucose uptake, with the 190-243 polypeptide fragment (consisting of a polypeptide with the sequence of SEQ ID No: 1) displaying the largest and most consistent influence on L6 myotube glucose uptake (1.77±0.23-fold change versus control; p=≦0.05).

rHDL Mediated Glucose Uptake is apoA-I Protein-Dependent

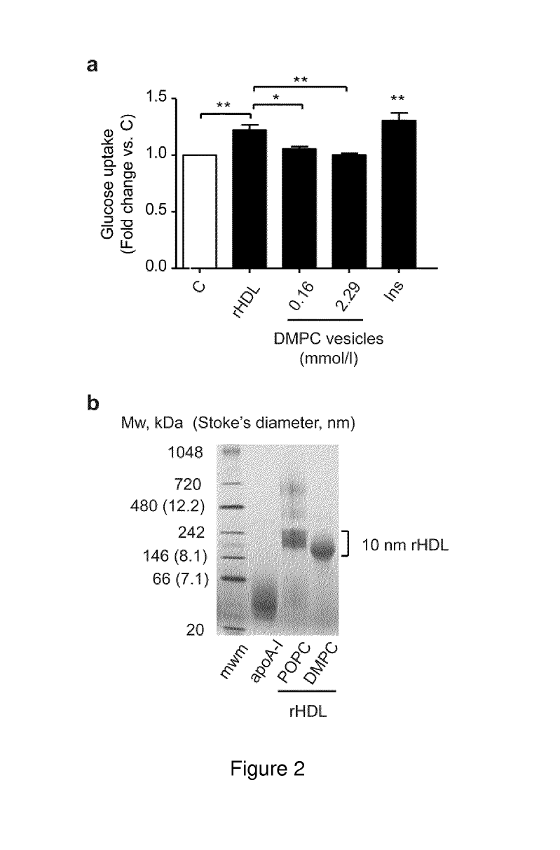

To test for the contribution of the constituent phospholipid to rHDL-induced glucose uptake in L6 myotubes, rHDL made with DMPC was also produced and compared to 100 nm DMPC vesicles containing no apoA-I protein. Stable 100 nm diameter vesicles can be synthesized with DMPC in the absence of apoA-I and was therefore used as a phospholipid control. The effect on the glucose uptake was tested as described above. Whereas rHDL particles synthesized from apoA-I (50 μg/ml) and DMPC (0.16 mmol/l) induced glucose uptake to a level similar to insulin-stimulated cells, incubation with empty DMPC vesicles (0.16 mmol/l equivalent to the lipid concentration in rHDL with 50 mg/ml apoA-I) did not induce glucose uptake. Even at a 14-fold higher concentration (2.29 mmol/l) DMPC vesicles had no influence on glucose uptake (FIG. 2a). To also test for the contribution of the constituent POPC phospholipid to rHDL-induced glucose uptake, rHDL made with POPC was compared with 100 nm POPC vesicles containing no apoA-I protein. Incubation with empty POPC vesicles (0.10 mmol/l) did not induce glucose uptake (FIG. 7c). The rHDL treatment induced a glucose uptake that was 2.3±0.39-fold p=≦0.05) over basal, which was similar to insulin stimulation (2.4±1.0-fold)(FIG. 7c).

Phosphorylation of AMPK and ACC, but not Akt, is Increased in L6 Myotubes Treated with rHDL

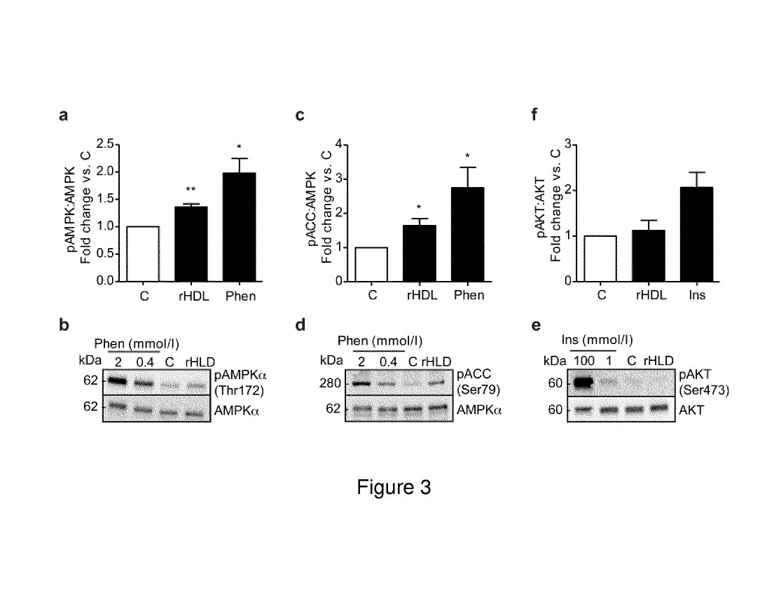

To dissect the effect of discoidal rHDL on a non-insulin dependent signaling pathway, we conducted western blotting using lysates from L6 myotubes incubated with rHDL. After 60 minutes of treatment (700 μg/ml apoA-I rHDL), L6 myotube lysates showed increased (1.36±0.071-fold; p=≦0.01) levels of phosphorylated AMPK (FIG. 3 a, b) and its downstream target ACC (1.64±0.26-fold; p=≦0.05) (FIG. 3 c, d). Phenformin was used as a positive control, inducing a 1.98±0.33-fold (p=≦0.05) and 2.7±0.74-fold (p=≦0.05) elevation of phosphorylated AMPK and ACC, respectively, at a concentration of 0.4 mmol/l. In contrast, Akt phosphorylation was unaffected by rHDL (1.12±0.38 S.D.-fold), while insulin (1 nmol/l) had a 2.06±0.47 S.D.-fold effect (FIG. 3 f, e). For clarity 1 nmol/l insulin (n=2) and 100 nmol/l insulin (n=3) (51±12.5-fold; p=≦0.01) was plotted in FIG. 3e. Representative immunoblots of those quantified for FIGS. 3a, c and f are given in FIGS. 3b, d and e, respectively.

Phosphorylation of AMPK but not Akt, is Increased in L6 Myotubes Treated with the 190-243 Polypeptide Fragment of Human ApoA-I (SEQ ID NO: 1).

The effect of the peptide fragment 190-243 of human Apo A-I (SEQ ID NO: 1) on a non-insulin dependent signaling pathway, was tested using lysates from L6 myotubes as described above for rHDL. Myotubes were serum starved for 4 hours then treated for 60 minutes with 2, 10 or 20 μmol/l of 190-243 peptide, 1 mmol/l phenformin, 100 nmol/l insulin, or control (C) followed by western blot analysis for phosphorylated AMPK (AMP-activated protein kinase) or phosphorylated Akt. Tubulin was used as loading control. Blots shown are representative of three independent experiments.

As can be seen from FIG. 8c, L6 myotube lysates showed increased levels of phosphorylated AMPK (FIG. 8c top panel). In contrast, Akt phosphorylation was unaffected by treatment with the peptide fragment 190-243 (FIG. 8c lower panel).

A person skilled in the art will appreciate that the effects on glucose uptake on myoblast cells and/or myotubes of any of the polypeptides according to the present invention having an amino acid sequence of SEQ ID NO: 1 to 1035 can be tested by using the methods of this example.

### Example 3

### Ex Vivo Study on Effects on Plasma Membrane GLUT4 Levels

To support the glucose uptake observations described in Example 2, the ability of rHDL and the 190-243 polypeptide fragment (SEQ ID NO:1) to increase the amount of GLUT4 in the plasma membrane was assessed by immunofluorescence microscopy of intact primary skeletal flexor digitorum brevis (FDB) muscle fibers, isolated from a transgenic mouse model with muscle-specific HA-GLUT4-GFP expression (Lizunov et al. (2012) Am J Physiol Endocrinol Metab 302: E950-960). The HA epitope present on the first exofacial loop of the HA-GLUT4-GFP construct allows detection of GLUT4 inserted into the plasma membrane. Intact FDB fibers were incubated ex vivo with rHDL followed by fixation and HA antibody labeling of non-permeabilized cells as described below.

### Preparation and Immunostaining of Primary Skeletal Muscle Fibers

Two to five transgenic mice (C57/Bl6; 10-14 weeks) with muscle specific HA-GLUT4-GFP expression (gift from S. Cushman) (Lizunov et al. (2012) Am J Physiol Endocrinol Metab 302: E950-960), were used for each condition. The animals were euthanized and flexor digitorum brevis (FDB) muscles dissected out and incubated with oxygenated Krebs-Henseleit carbonate Hepes (KRBH) buffer (6 mmol/l KCl, 1 mmol/l Na₂HPO₄, 0.2 mmol/l NaHPO₄, 1.4 mmol/l MgSO₄, 1 mmol/l CaCl₂, 128 mmol/l NaCl, 10 mmol/l HEPES pH 7.4) with 0.5% (w/v) bovine serum albumin. After dissection, muscles were continually oxygenated with 95% O₂/5% CO₂ and incubated at 37° C. for 1-2 hours in a water bath with slow shaking. After incubation, muscles were washed 3 times with oxygenated KRBH, and were then either treated with insulin (100 nmol/l), apoA-I (full length lipid free), rHDL or apoA-I 190-243 polypeptide fragment, or kept basal for 1 hour. After stimulation, basal (non-stimulated) and stimulated muscles were fixed for 10 min with 4% paraformaldehyde in PBS, washed 3 times with PBS containing 1% BSA, and incubated for 30-60 min with anti-HA (Covance) followed by 30 min with fluorescently labeled secondary antibodies Alexa-647 (Invitrogen).

### Confocal Microscopy

Fixed cells were imaged using a confocal LSM 510 microscope (Zeiss) using a 40× objective, NA 1.3, using BP 505-530 and LP 650. Images were collected with the LSM software.

Both insulin and rHDL treatment induced translocation of GLUT4 into the sarcolemma plasma membrane as detected by HA signal (FIG. 1d upper panel). The lower panel in FIG. 1d displays total GLUT4 detected by GFP signal merged with the HA signal in non-stimulated and stimulated muscle fibers. Due to steric hindrance, labeling of the transverse tubules are limited and therefore the plasma membrane GLUT4 translocation is assessed only at the sarcolemma. Please also refer to FIG. 7d which shows an alternative representation of the same data.

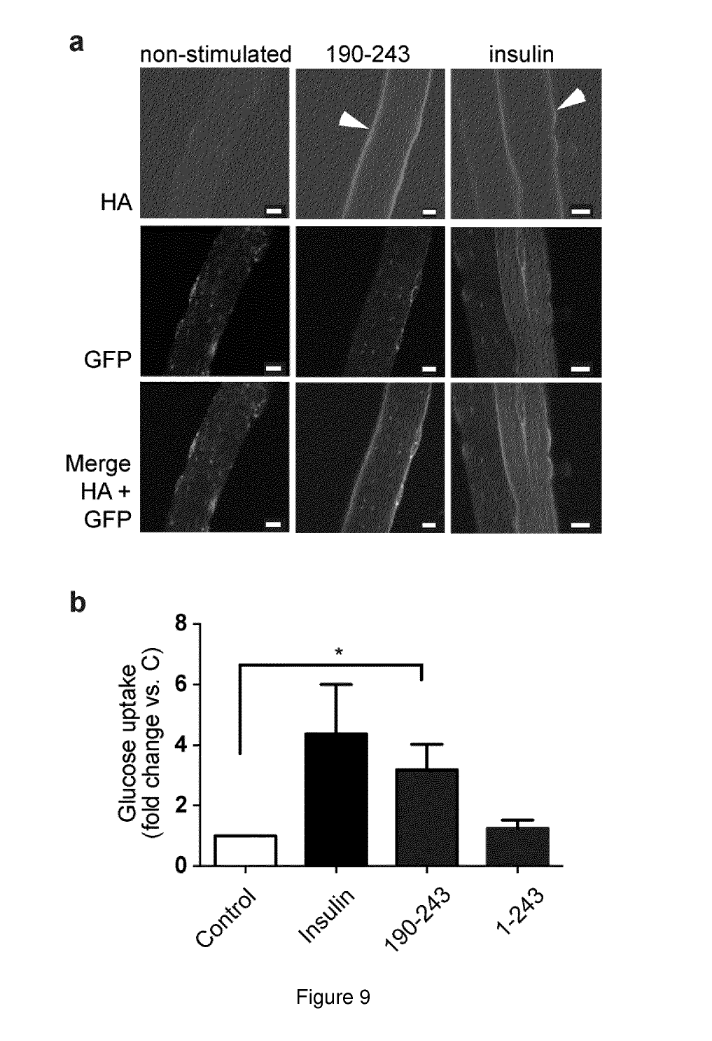

To verify this observation, confocal immunofluorescence imaging of FDB fibers labeled with HA-antibody was performed after ex vivo incubation with the 190-243 polypeptide fragment. In relation to basal conditions, these images show greater membrane levels of GLUT4 protein in response to the 190-243 polypeptide fragment (FIG. 4c), thus supporting the findings in FIG. 4b. Please also refer to FIG. 9a which shows an alternative representation of the same data.

A person skilled in the art will appreciate that the effects on plasma membrane GLUT4 levels of any of the polypeptides according to the present invention having an amino acid sequence of SEQ ID NO: 1 to 1035 can be tested by using the methods of this example.

### Example 4

### Structural Characterisation of Polypeptides According to the Invention

The polypeptides of the present invention having the SEQ ID NO: 1 to 1035 may be characterized with respect to molecular mass, secondary and tertiary helical content and other characteristics of the polypeptides by use of methods known in the art. For example by using any one of the methods as described below for the polypeptides consisting of amino acids 1-243, and 190-243 of the human apoA-I protein. Note that the 190-243 polypeptide fragment consists of amino acids 190-243 of the human apoA-I protein and has the sequence of SEQ ID NO:1.

### Blue-Native Polyacrylamide Gel Electrophoresis (PAGE)

Blue-native PAGE was performed using methods known in the art on all rHDL preparations to confirm the formation of 10 nm diameter discoidal apoA-I dimers. FIG. 2b is a representative coomassie stained gel that shows monomeric lipid-free apoA-I (˜28 kDa) and the size similarity between POPC and DMPC rHDL (both ˜10 nm diameter).

### Circular Dichroism (CD) Spectroscopy

CD measurements were performed on a Jasco J-810 spectropolarimeter equipped with a Jasco CDF-4265 Peltier set to 25° C. ApoA-I (full-length and the 190-234 polypeptide fragment) was diluted to 0.2 mg/ml in PBS (final concentration was 25 mmol/l phosphate, 25 mmol/l NaCl, pH 7.4), placed in a 0.1 mm quartz cuvette and, after extensive purging with nitrogen, scanned in the region 200 to 260 nm (scan speed was 20 nm/min). Averages of five scans were baseline-subtracted (PBS; 25 mmol/l phosphate, 25 mmol/l NaCl) and the alpha-helical content was calculated from the molar ellipticity at 222 nm as previously described (Petrlova et al. (2012) J Lipid Res 53: 390-398).

Structural Conformation of Lipid-Free 190-243 Fragment Resembles the Conformation of Lipid Bound apoA-I

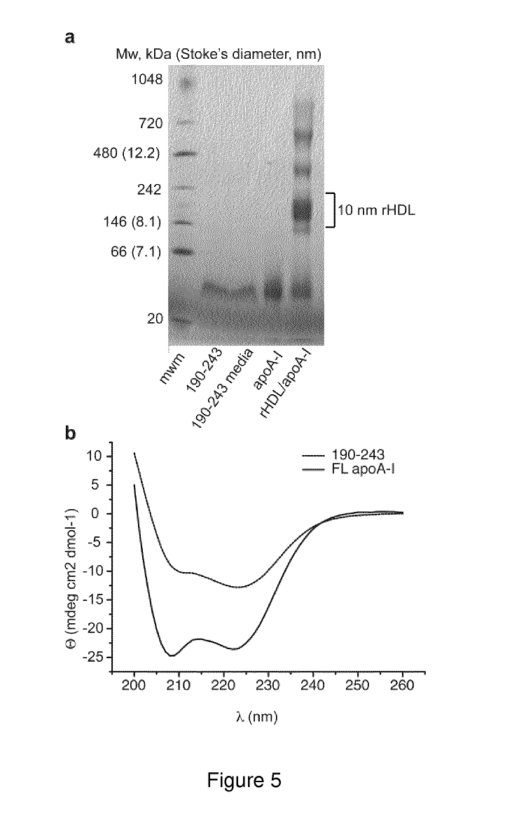

It was hypothesised that binding of cellular lipids to the 190-243 polypeptide fragment upon incubation with L6 myotubes and FDB fibers may be necessary for its glucose uptake-inducing effect. It is known that the 190-234 polypeptide fragment can form discoidal particles by solubilisation of lipid in solution (Vedhachalan et al. (2010) J Biol Chem 285:31965-31973), which can be visualised as oligomers on native PAGE. To assess oligomer formation, indicative of lipid binding, purified 190-243 polypeptide fragment and conditioned media from cells treated with the purified 190-243 polypeptide fragment for 1 hour were run on a blue native PAGE and coomassie stained (FIG. 5a). Under both conditions the purified 190-243 polypeptide fragment appeared as a single band at approximately 40 kDa corresponding to a tetramer. Lipid-free apoA-I and rHDL were included on the gel as a full length protein monomer and dimer reference.

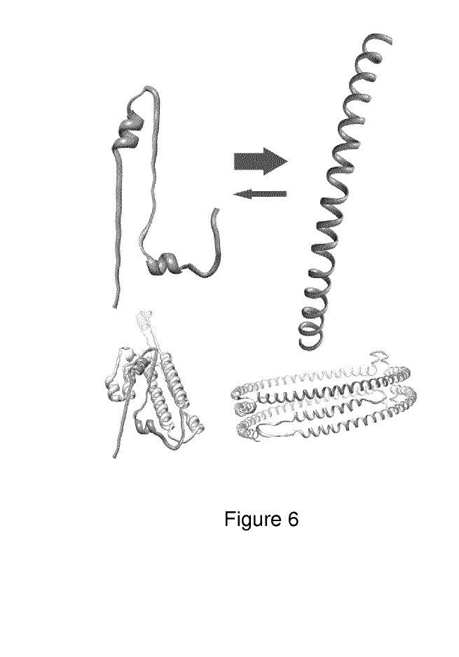

As depicted in FIG. 1a the structure of apoA-I in the apo state is significantly different from the structural organization of the same protein in rHDL particles. This structural transition of the lipid-binding process involves a major increase in alpha-helical secondary structure (from about 44% in the apo-state to 78% alpha-helical secondary structure in discoidal HDL (Saito et al. (2004) J Biol Chem 279:20974-20981). As rHDL appears to be more potent in stimulation of glucose uptake in myotubes than the corresponding lipid-free protein (compare glucose uptake levels in FIGS. 1b and 1c) we speculated that a lipid-free 190-243 polypeptide fragment may adopt an amphiphatic alpha-helix in solution. To investigate this, CD spectroscopy spectra were obtained for the 190-243 polypeptide fragment and full-length protein for comparison (FIG. 5). The helical content was estimated from their molar ellipticities at 222 nm to be 28% and 43% for the 190-243 polypeptide fragment and full-length apoA-I, respectively, suggesting an increase in helical structure of the 190-243 polypeptide fragment. FIG. 6 shows a structural overview of the proposed helical structure of the 190-243 polypeptide fragment.

### Example 5

### Studies of the Effects of apoA-I Polypeptides on Glucose Uptake in Adipose Cells

### Adipose Cell Preparations

Adipose cells were prepared as previously described (Rodbell (1964) J Biol Chem. 239: 375-80). In brief, epidydymal fat pads were minced and digested in Krebs-Ringer-bicarbonate-HEPES (KRBH) buffer, pH 7.4, containing 10 mM sodium bicarbonate, 30 mM HEPES, 200 nM adenosine, 1% bovine serum albumin (BSA) with 1 mg/ml collagenase. After digestion, the cells were filtered through nylon mesh (250 μm) and washed with KRBH-buffer.

### Glucose Uptake in Adipose Cells

Isolated rat adipocytes were treated for 60 min with insulin (100 nmol/l), 190-243 polypeptide fragment (having the amino acid sequence consisting of SEQ ID NO:1) (30 μg/ml) or 1-243 human apoA-I protein (150 μg/ml) followed by glucose uptake measurement by addition of UC14 tracer glucose (Perkin Elmer) (30 min). The uptake was terminated by centrifugation through dinonyl phtalate oil. Incubation with Cytochalasin B was used to subtract unspecific background binding. Amount of glucose was detected as count radioactivity measured by Beta CounterThe amount of glucose transported was measured using an isotope labeled UC14-assay.

As can be seen from FIG. 9b the glucose uptake was significantly induced by incubation of the adipocytes with insulin (100 nmol/l) and with 190-243 polypeptide fragment (30 μg/ml).

A person skilled in the art will appreciate that the effects on induced glucose uptake in adipocytes of any of the polypeptides according to the present invention having an amino acid sequence of SEQ ID NO: 1 to 1035 can be tested by using the methods of this example.

### Example 6

### In Vivo Effects of Treatment with Apo-AI Derived Peptides

Study of the in vivo effects of short treatments with human apoA-I 190-243 polypeptide fragment (SEQ ID NO: 1), and fragments thereof in a high-fat diet fed (HFD) mouse model

### Animals and Diets

Male C57BL/6 mice (Taconic) were used at 9-12 weeks of age. Animals were on a 12 hour light cycle with non-restricted food and water. The HFD animals were fed an HFD (D12492, 60% fat content; Research Diets) for 2 weeks. All animal procedures were approved by the Malmo/Lund Committee for Animal Experiment Ethics, Lund, Sweden.

### Polypeptides

The following peptide fragments of human ApoA-I were tested:

### 54-mer Fragment

A fragment consisting of amino acids 190-243 of human ApoA-I (SEQ ID NO: 1)

### 27-mer Fragments

A fragment consisting of amino acids 190-216 of human ApoA-I (SEQ ID NO: 7)

A fragment consisting of amino acids 217-243 of human ApoA-I (SEQ ID NO: 8

### 23-mer Fragments

A fragment consisting of amino acids 204-226 of human ApoA-I (SEQ ID NO: 518)

### 18-mer Fragments

A fragment consisting of amino acids 190-207 of human ApoA-I (SEQ ID NO: 2)

A fragment consisting of amino acids 208-225 of human ApoA-I (SEQ ID NO: 3)

A fragment consisting of amino acids 226-243 of human ApoA-I (SEQ ID NO: 4)

### Glucose Tolerance Test

HFD mice fasted overnight (12 hours) were injected intra peritoneal (i.p.) with apoA-I peptides as listed above (7 mg/ml in PBS pH 7.4 for the 54-mer fragment and 6-7 mg/kg for other fragments in PBS, pH 7.4; control animals received NaCl) and this was followed by collection of serum samples at the indicated times. Glucose (50 mg/mouse) was injected i.p. 3 hours after treatments, followed by collection of serum samples at the indicated times. Blood glucose levels were measured (OnetouchUltra2; Lifescan), and insulin levels were assayed in serum using ELISA (Mercodia).

### Statistical Analysis

All data are displayed as mean±SEM unless indicated otherwise. Where appropriate, analysis was performed by two-tailed student's t test or one-way ANOVA with Bonferroni's post hoc test using Microsoft Excel and Graph Pad Prism software. p=≦0.05 was considered significant.

### Results

As can be seen in FIG. 10, treatment with the 190-243 polypeptide fragment (SEQ ID NO: 1) significantly improved insulin secretion (FIG. 10c,d) and blood glucose clearance (FIG. 10a,b) in the glucose tolerance test, with an efficiency exceeding that of control animals. The area under curve (AUC) measurements show that total glucose experienced by the peptide treated animals is reduced with about 40% during the time period of the test (120 minutes). Similarly the glucose-stimulated insulin secretion is increased with about 60% during the time period of the test (120 minutes).

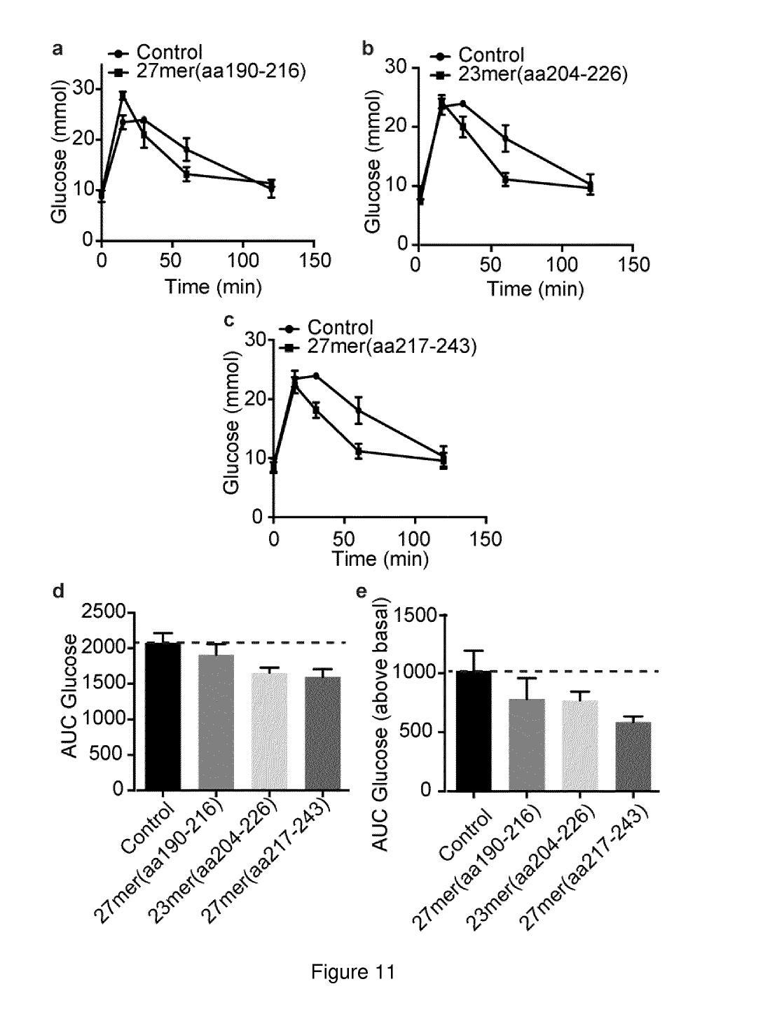

As can be seen in FIG. 11, the tested peptides (23 or 27-mers) within the 190-243 region of apoA-I had an influence on glucose tolerance capacity. Peptides 23-mer (aa204-226, SEQ ID NO: 518) and 27-mer (aa217-243, SEQ ID NO: 8) showed particular increased capacity to clear blood glucose. The results show that shorter peptides based on the 190-243 polypeptide fragment are competent in improving glucose tolerance.

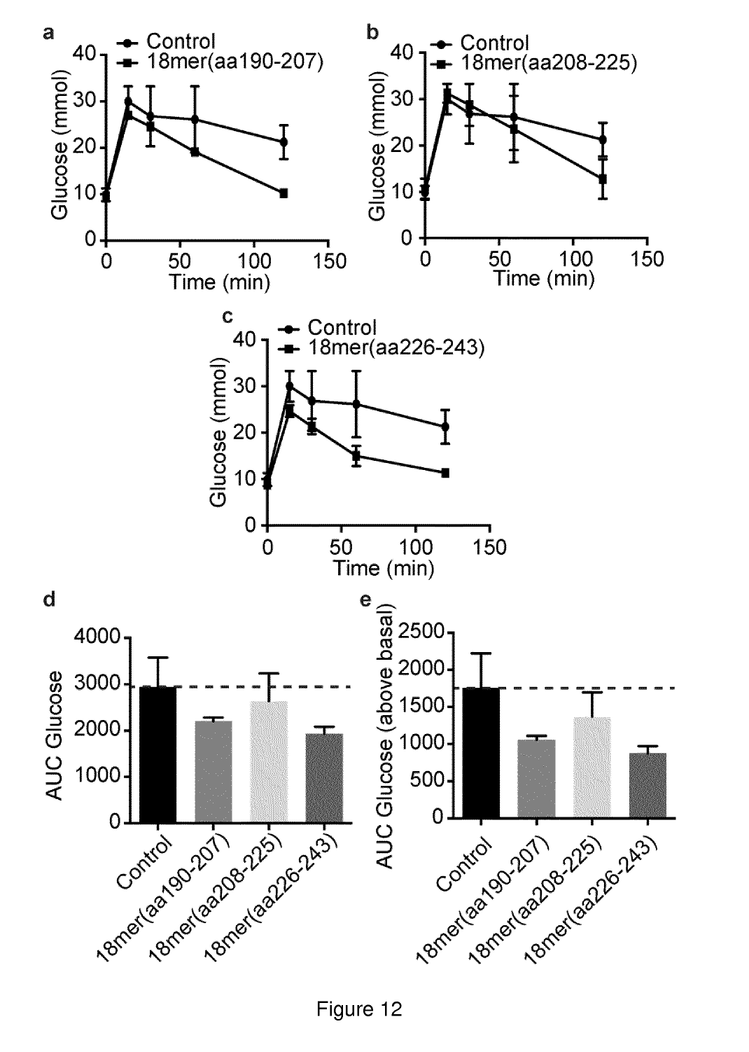

As can be seen from FIG. 12, peptides (18-mers) within the 190-243 region of apoA-I had influence on glucose tolerance capacity. Peptides 18-mer (aa190-207, SEQ ID NO: 2 and 18-mer (aa226-243, SEQ ID NO: 4) showed particular increased capacity to clear blood glucose. The results show that shorter peptides based on the 190-243 polypeptide fragment are competent in improving glucose tolerance.

A person skilled in the art will appreciate that the effects on improved glucose tolerance of any of the polypeptides according to the present invention having an amino acid sequence of SEQ ID NO: 1 to 1035 can be tested by using the methods of this example.

### Example 7

### Treatment of Insulin-Resistant Patients

Humans patients suffering from insulin-resistance are treated with either saline solution (control group) or a saline solution of 5 mg/kg bodyweight a polypeptide consisting of the human apoA-I 190-243 peptide fragment having the amino acid sequence of SEQ ID NO: 1 injected subcutaneously within 30-60 minutes before a meal.

The purpose of the treatment is to induce cell glucose uptake in the treated patients and decrease fastening plasma glucose levels thus having a positive effect on the glucose metabolism. Depending on the individual's response to the medication 2-5 bolus injections or alternatively an infusion may be required per day.

The effects on the glucose uptake is measured by following the plasma glucose at selected time-points prior to and after meals using a glucose oxidase assay such as YSI 2300 STAT Plus; YSI, Yellow Springs, Ohio

### Example 8

### Treatment of Metabolic Syndrome

A human patient diagnosed with at least three of the criteria of metabolic syndrome (abdominal obesity, HDL cholesterol of less than 40 mg/dl for men and less than 50 mg/dl for women, blood pressure of at least 130/85 mm Hg, fasting plasma glucose levels of at least 110 mg/dl, and plasma triglycerides of at least 150 mg/dl) is treated during a 8-week period with subcutaneously injected ApoA-I 190-243 polypeptide (SEQ ID NO: 1) 2-5 times daily within 30-60 minutes before a meal.

The purpose of the treatment is to induce cell glucose uptake in the treated patients, thus having a positive effect on the signs of metabolic syndrome, for example by decreasing the blood pressure, fasting plasma glucose levels or plasma triglycerides levels, and increasing the HDL cholesterol. Depending on the individual's response to the medication 2-5 bolus injections or alternatively an infusion may be required per day.

1. An isolated polypeptide for use in a method of treatment or prevention of diseases characterised by hyperglycaemia and/or insulin resistance, said polypeptide comprising an amino acid sequence selected from the group consisting of:
a) the amino acid sequence of SEQ ID NO: 1;
b) a biologically active sequence variant of a), wherein the variant has at least 70% sequence identity to SEQ ID NO:1; and
c) a biologically active fragment of a) or b) wherein the fragment comprises at least 10 consecutive amino acids of SEQ ID NO: 1
wherein said polypeptide has a length that is less than 100 amino acids, and
wherein said biological activity is induction of glucose uptake in cells.

2. The polypeptide according to claim 1, wherein said polypeptide has at least 70% sequence identity to the amino acid sequence of SEQ ID NO: 1, more preferably at least 75%, more preferably at least 80%, more preferably at least 85%, more preferably 90%, more preferably at least 95%, more preferably at least 98%, more preferably at least 99% sequence identity to the amino acid sequence of SEQ ID NO: 1.

3. The polypeptide according to any one of the preceding claims, wherein in said sequence variant any one amino acid residue has been altered to another amino acid residue, provided that no more than 15 amino acids have been so altered, such as wherein no more than 14 amino acids have been so altered, e.g. wherein no more than 13 amino acids have been so altered, such as wherein no more than 12 amino acids have been so altered, e.g. wherein no more than 11 amino acids have been so altered, such as wherein no more than 10 amino acids have been so altered, e.g. wherein no more than 9 amino acids have been so altered, such as wherein no more than 8 amino acids have been so altered, e.g. wherein no more than 7 amino acids have been so altered, such as wherein no more than 6 amino acids have been so altered, e.g. wherein no more than 5 amino acids have been so altered, such as wherein no more than 4 amino acids have been so altered, e.g. wherein no more than 3 amino acids have been so altered, such as wherein no more than 2 amino acids have been so altered, e.g. wherein no more than 1 amino acid has been so altered in relation to said SEQ ID NO: 1.

4. The polypeptide according to any one of the preceding claims, wherein said polypeptide consists essentially of said amino acid sequence.

5. The polypeptide according to any one of the preceding claims, wherein said polypeptide consists of said amino acid sequence.

6. The polypeptide according to any one of the preceding claims, wherein said polypeptide is a variant polypeptide described therein, wherein any amino acid has been altered to provide a conservative substitution relative to the amino acid sequence of SEQ ID NO: 1.

7. The polypeptide according to any one of the preceding claims, wherein the said polypeptide comprises conserved amino acid residues at positions Ala 1, Glu 2, Tyr 3, His 4, Ala 5, Lys 6, Ala 7, Glu 9, Leu 11, Leu 14, Glu 16, Lys 17, Pro 20, Leu 22, Glu 23, Asp 24, Leu 25, Arg 26, Leu 29, Pro 31, Glu 34, Lys 37, Glu 45, Glu 46, Lys 49, Lys 50, Leu 51, Gln 54 relative to the amino acid sequence of SEQ ID NO:1.

8. The polypeptide according to any one of the preceding claims, wherein the said polypeptide comprises conserved amino acid residues at positions Glu 2, Tyr 3, Leu 11, Leu 14, Glu 16, Lys 17, Pro 20, Asp 24, Leu 29, Pro 31, Glu 34, Lys 37, Glu 45 relative to the amino acid sequence of SEQ ID NO:1.

9. The polypeptide according to any one of the preceding claims, wherein said disease characterised by hyperglycaemia is a metabolic disease.

10. The polypeptide according to any one of the preceding claims, wherein said disease is type II diabetes

11. The polypeptide according to any one of the preceding claims, wherein said disease is insulin resistance.

12. The polypeptide according to any one of the preceding claims, wherein said disease is type I diabetes.

13. The polypeptide according to any one of the preceding claims, wherein said disease is selected from the group consisting of endocrine and metabolic diseases and obesity.

14. The polypeptide according to any one of the preceding claims, wherein said disease is an endocrine and metabolic disease selected from the group consisting of non-insulin-dependent diabetes mellitus such as adult-onset, maturity-onset, nonketotic, stable, type II and non-insulin-dependent diabetes of the young.

15. The polypeptide according to any one of the preceding claims, wherein said disease is an endocrine and metabolic disease selected from the group consisting of polycystic ovarian syndrome such as Sclerocystic ovary syndrome and Stein-Leventhal syndrome.

16. The polypeptide according to any one of the preceding claims, wherein said disease is an endocrine and metabolic disease such as obesity e.g. obesity due to excess calory intake.

17. The polypeptide according to any one of the preceding claims, wherein said disease is abnormal glucose tolerance test including chemical and latent diabetes; impaired glucose tolerance; and/or prediabetes.

18. The polypeptide according to any one of the preceding claims, wherein said disease is a metabolic disease selected from the group consisting of: metabolic syndrome, insulin resistance, glucose intolerance, hyperglycemia, type I diabetes, type II diabetes, hyperlipidemia, hypertriglyceridemia, hypercholesterolemia, dyslipidemia, and polycystic ovary syndrome.

19. The polypeptide according to any of the preceding claims, wherein said metabolic diseases are caused by insulin resistance selected from the group of insulin resistance in the liver, insulin resistance in the skeletal muscles and/or insulin resistance in adipose tissue.

20. The polypeptide according to any one of the preceding claims, wherein the subject to be treated is a mammal, preferably a primate, more preferably a human being.

21. The polypeptide according to any one of the preceding claims, wherein said treatment results in increased uptake of glucose in myeloid and/or adipose tissue.

22. The polypeptide according to any one of the preceding claims, wherein said treatment results in disease modification in at least a subset of the treated subjects.

23. The polypeptide according to any one of the preceding claims, wherein the polypeptide is administered or adapted for administration enterally, topically, parenterally or as part of a sustained release implant.

24. The polypeptide according to any one of the preceding claims, wherein the parenteral administration is intravenous, subcutaneous, intramuscular, intracranial or intraperitoneal.

25. The polypeptide according to any one of the preceding claims, wherein the enteral administration is oral, rectal, or buccal.

26. The polypeptide according to any one of the preceding claims, wherein the topical administration is dermal, epicutaneous, vaginal, intravesical, pulmonary, intranasal, intratracheal or as eye drops.

27. The polypeptide according to any one of the preceding claims, wherein the polypeptide is administered or adapted for administration subcutaneously or intravenously.

28. The polypeptide according to any one of the preceding claims, wherein the polypeptide is administered or adapted for administration in dosages of 1 μg/kg-10,000 μg/kg body weight, such as 1 μg/kg-7,500 μg/kg, such as 1 μg/kg-5,000 μg/kg, such as 1 μg/kg-2,000 μg/kg, such as 1 μg/kg-1,000 μg/kg, such as 1 μg/kg-700 μg/kg, such as 5 μg/kg-500 μg/kg, such as 10 μg/kg to 100 μg/kg body weight.

29. The polypeptide according to any one of the preceding claims, wherein said administration is repeated daily.

30. The polypeptide according to any one of the preceding claims, wherein said administration is repeated at least 1-3 times weekly, such as 2-5 times weekly, such as 3-6 times weekly.

31. The polypeptide according to any one of the preceding claims, wherein said administration is repeated 1 to 8 times daily, such as 2 to 5 times daily.

32. The isolated polypeptide according to any one of the preceding claims wherein said polypeptide comprises an amino acid sequence selected from the group consisting of an amino acid sequence of SEQ ID NOs: 1 to 1035.

33. The isolated polypeptide according to any one of the preceding claims wherein said polypeptide consists essentially of an amino acid sequence selected from the group consisting of an amino acid sequence of SEQ ID NOs: 1 to 1035.

34. The isolated polypeptide according to any one of the preceding claims wherein said polypeptide consists of an amino acid sequence selected from the group consisting of an amino acid sequence of SEQ ID NOs: 1 to 1035.

35. The isolated polypeptide according to any one of the preceding claims wherein said polypeptide comprises an amino acid sequence selected from the group consisting of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

36. The isolated polypeptide according to any one of the preceding claims wherein said polypeptide consists essentially of an amino acid sequence selected from the group consisting of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

37. The isolated polypeptide according to any one of the preceding claims wherein said polypeptide consists of an amino acid sequence selected from the group consisting of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

38. The isolated polypeptide according to any one of the preceding claims wherein said polypeptide comprises an amino acid sequence selected from the group consisting of SEQ ID NO: 1, SEQ ID NO: 2, SEQ ID NO: 3, SEQ ID NO: 4, SEQ ID NO: 7, SEQ ID NO: 8 and SEQ ID NO: 518.

39. The isolated polypeptide according to any one of the preceding claims wherein said polypeptide consists essentially of an amino acid sequence selected from the group consisting of SEQ ID NO: 1, SEQ ID NO: 2, SEQ ID NO: 3, SEQ ID NO: 4, SEQ ID NO: 7, SEQ ID NO: 8 and SEQ ID NO: 518.

40. The isolated polypeptide according to any one of the preceding claims wherein said polypeptide consists of an amino acid sequence selected from the group consisting of SEQ ID NO: 1, SEQ ID NO: 2, SEQ ID NO: 3, SEQ ID NO: 4, SEQ ID NO: 7, SEQ ID NO: 8 and SEQ ID NO: 518.

41. The isolated polypeptide according to any one of the preceding claims wherein said polypeptide comprises an amino acid sequence selected from the group consisting of SEQ ID NO: 1, SEQ ID NO: 2, SEQ ID NO: 3, SEQ ID NO: 4, SEQ ID NO: 7 and SEQ ID NO: 8.

42. The isolated polypeptide according to any one of the preceding claims wherein said polypeptide consists essentially of an amino acid sequence selected from the group consisting of SEQ ID NO: 1, SEQ ID NO: 2, SEQ ID NO: 3, SEQ ID NO: 4, SEQ ID NO: 7 and SEQ ID NO: 8.

43. The isolated polypeptide according to any one of the preceding claims wherein said polypeptide consists of an amino acid sequence selected from the group consisting of SEQ ID NO: 1, SEQ ID NO: 2, SEQ ID NO: 3, SEQ ID NO: 4, SEQ ID NO: 7, SEQ ID NO: 8 and SEQ ID NO: 518.

44. The isolated polypeptide according to any one of the preceding claims wherein said polypeptide comprises the amino acid sequence of SEQ ID NO: 1.

45. The isolated polypeptide according to any one of the preceding claims wherein said polypeptide consists essentially of the amino acid sequence of SEQ ID NO: 1.

46. The isolated polypeptide according to any one of the preceding claims wherein said polypeptide consists of the amino acid sequence of SEQ ID NO: 1.

47. The isolated polypeptide according to any one of the preceding claims wherein said polypeptide is conjugated to a polypeptide, and/or another molecule, such as a linker that is not derived from human apolipoprotein A-I (apoA-I).

48. The isolated polypeptide according to any one of the preceding claims wherein said polypeptide comprises one or more amino acid substitutions, additions or deletions compared to any one of SEQ ID NOs: 1 to 1035, which substitutions, additions or deletions further increases the solubility of the peptide compared to one or more of the amino acid sequences of SEQ ID NOs: 1 to 1035.

49. The isolated polypeptide according to any one of the preceding claims wherein said polypeptide comprises one or more amino acid substitutions, additions or deletions compared to any one of SEQ ID NOs: 1 to 1035 which increases the glucose uptake of a mammalian cell compared to the human apolipoprotein A-I.

50. The isolated polypeptide according to any one of the preceding claims wherein said polypeptide has one or more amino acid substitutions, additions or deletions compared to any one of SEQ ID NOs: 1 to 1035 which increases the expression of the said polypeptide in a recombinant host cell, or in an in vitro translation system, or facilitates production in chemical synthesis compared to one or more of the amino acid sequences of any one of SEQ ID NOs: 1 to 1035.

51. The polypeptide according to any one of the preceding claims, wherein the polypeptide is chemically modified in order to increase its half-life when administered to a patient, in particular its plasma half-life.

52. The polypeptide according to any one of the preceding claims, wherein said polypeptide further comprises a moiety conjugated to said polypeptide, thus generating a moiety-conjugated polypeptide

53. The polypeptide according to any one of the preceding claims, wherein the moiety-conjugated polypeptide has a plasma and/or serum half-life being longer than the plasma and/or serum half-life of the non-moiety conjugated polypeptide.

54. The polypeptide according to any one of the preceding claims, wherein the moiety conjugated to the polypeptide is one or more type of moieties selected from the group consisting of albumin, fatty acids, polyethylene glycol (PEG), acylation groups, antibodies and antibody fragments.

55. The polypeptide according to any one of the preceding claims, wherein the polypeptide and the moiety are conjugated to each-other by a linker.

56. The polypeptide according to any one of the preceding claims, wherein more than one moiety is conjugated to the polypeptide.

57. The polypeptide according to any one of the preceding claims, wherein any one of said SEQ ID NOs: 1 to 1035 has the therapeutic effect.

58. The polypeptide according to any one of the preceding claims, wherein said polypeptide does not comprise GLP-1 or a biologically active fragment or variant of GLP-1.

59. An isolated polypeptide consisting essentially of an amino acid sequence selected from the group consisting of the amino acid sequence of SEQ ID NO: 3, 4 or 6.

60. An isolated polypeptide consisting of an amino acid sequence selected from the group consisting of the amino acid sequence of SEQ ID NO: 3, 4 or 6.

61. An isolated polypeptide consisting essentially of an amino acid sequence selected from the group consisting of the amino acid sequence of SEQ ID NOs: 9 to 1035.

62. An isolated polypeptide consisting of an amino acid sequence selected from the group consisting of the amino acid sequence of SEQ ID NOs: 9 to 1035.

63. The polypeptide according to any one of the preceding claims, wherein any one amino acid residue in said amino acid sequence has been altered to the corresponding D-amino acid.

64. The polypeptide according to any one of the preceding claims, wherein all amino acid residues in said amino acid sequence has been altered to the corresponding D-amino acids.

65. An isolated polynucleotide which upon expression encodes the polypeptide according to any one of claims 59 to 62.

66. A vector, such as an expression vector, comprising the polynucleotide according to claim 65.

67. A host cell comprising the polynucleotide according to claim 65, and/or the vector according to any one of claims 66 to 67.

68. The isolated polypeptide according to any one of claims 59 to 64, the isolated polynucleotide according to claim 65, the vector according to claim 66, or the host cell according to claim 67, for use as a medicament.

69. A pharmaceutical composition comprising an isolated polypeptide according to any one of claims 59 to 64, the isolated polynucleotide according to claim 65, the vector according to claim 66, or the host cell according to claim 67.

70. The pharmaceutical composition according to claim 69 further comprising a second active ingredient for treatment of metabolic disease and/or cardiovascular disease.

71. The pharmaceutical composition according to claim 70 wherein said second active ingredient is a compound used for the treatment of metabolic diseases, or for the prophylactic treatment of a mammal facing the risk of developing a metabolic disease.

72. The pharmaceutical composition according to any one of claims 69 to 71 wherein said second active ingredient is a compound used for treatment of diabetes.

73. The pharmaceutical composition according to any one of claims 69 to 72 wherein said second active ingredient is a compound selected from the group consisting of insulin, a derivative of insulin, metformin or derivatives thereof.

74. The pharmaceutical composition according to any one of claims 69 to 73, wherein said second active ingredient is a compound selected from the group consisting of diuretics, angiotensin-converting enzyme (ACE) inhibitors, beta blockers, blood thinners such as aspirin; and cholesterol-lowering drugs such as statins or fibrates.

75. The pharmaceutical composition according to any one of claims 69 to 74, wherein said second active ingredient is a compound selected from the group consisting of of Insulin, Exenatide, Exenatide Extended Release, Liraglutide, Pramlintide; Sulfonylureas, Biguanides, Meglitinides, Thiazolidinediones, DPP-4 inhibitors, SGLT2 Inhibitors, Alpha-glucosidase and Bile Acid Sequestrants inhibitors.

76. The isolated polypeptide according to any one of claims 59 to 62, the isolated polynucleotide according to claim 65, the vector according to claim 66, or the host cell according to claim 67, for use in a method of treatment or prevention of diseases characterised by hyperglycaemia and/or insulin resistance.

77. An isolated polynucleotide for use in a method of treatment or prevention of diseases characterised by hyperglycaemia and/or insulin resistance, said polynucleotide comprising a nucleic acid sequence which upon expression encodes a polypeptide as defined in any one of claims 1 to 64.

78. A vector for use in a method of treatment or prevention of diseases characterised by hyperglycaemia and/or insulin resistance, said vector comprising a polynucleotide comprising a nucleic acid sequence which upon expression encodes a polypeptide as defined in any one of claims 1 to 64.

79. The vector according to claim 78, further comprising a promoter operably linked to the polynucleotide.

80. The vector according to any of the preceding claims 78 to 79, wherein the vector is selected form the group consisting of alphavirus, adenovirus, adeno associated virus, baculovirus, HSV, coronavirus, Bovine papilloma virus, and Mo-MLV, preferably adeno associated virus.

81. An isolated host cell for use in a method of treatment or prevention of diseases characterised by hyperglycaemia and/or insulin resistance, wherein said cell is transformed or transduced with the polynucleotide according to claim 67 and/or the vector of claims 78 to 80.

82. The cell according to claim 81, wherein said cell is a human cell.

83. The cell according to any one of claims 81 and 82, wherein the cell is selected from the group consisting of stem cells, muscle cells, hepatocytes, adipocytes and cells of the pancreas such as α cells, β cells and δ cells.

84. The cell according to claim 83 wherein said host cell is selected from the group consisting of CHO, CHO-K1, HEI193T, HEK293, COS, HiB5, RN33b and BHK cells.

85. Use of an agent selected from the group consisting of:
a) an isolated polypeptide as defined in any one of claims 1 to 64;
b) an isolated polynucleotide as defined in claim 77;
c) a vector as defined in any one of claims 78 to 80; and
d) an isolated cell as defined in any one of claims 81 to 84,
for the preparation of a medicament for the treatment and/or prevention of diseases characterised by hyperglycaemia and/or insulin resistance.

86. Use of an agent selected from the group consisting of:
a) an isolated polypeptide consisting of less than 100 amino acid residues and comprising:
i) the amino acid sequence of SEQ ID NO: 1; or
ii) a biologically active sequence variant of the amino acid sequence of i) wherein the variant has at least 70% sequence identity to said SEQ ID NO: 1,
b) a nucleic acid sequence encoding a polypeptide as defined in a);
c) a vector comprising the nucleic acid molecule as defined in b),
d) an isolated host cell transformed or transduced with the nucleic acid of b) or the vector of c),
for the preparation of a medicament for the treatment and/or prevention of diseases characterised by hyperglycaemia and/or insulin resistance.

87. A method for treatment of diseases characterised by hyperglycaemia and/or insulin resistance, said method comprising administering to an individual in need thereof a therapeutically effective amount of an agent selected from the group consisting of:
a) an isolated polypeptide consisting of less than 100 amino acid residues and comprising:
i) the amino acid sequence of SEQ ID NO: 1; or
ii) a biologically active sequence variant of the amino acid sequence of i) wherein the variant has at least 70% sequence identity to said SEQ ID NO: 1,
iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c),

88. The use according to claim 86, or the method according to claim 87, wherein said polypeptide is as defined in any one of the preceding claims.

89. The use according to claim 86, or the method according to claim 87, wherein said polypeptide is as defined in any one of the preceding claims.

90. A method for reducing blood glucose, the method comprising contacting a mammal with an effective amount of an agent selected from the group consisting of:
a) an isolated polypeptide consisting of less than 100 amino acid residues and comprising:
i) the amino acid sequence of SEQ ID NO: 1; or
ii) a biologically active sequence variant of the amino acid sequence of i) wherein the variant has at least 70% sequence identity to said SEQ ID NO: 1,
iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c).

91. A method for increasing insulin secretion, the method comprising contacting a mammal with an effective amount of an agent selected from the group consisting of:
a) an isolated polypeptide consisting of less than 100 amino acid residues and comprising:
i) the amino acid sequence of SEQ ID NO: 1; or
ii) a biologically active sequence variant of the amino acid sequence of i) wherein the variant has at least 70% sequence identity to said SEQ ID NO: 1,
iii) a biologically active fragment of at least 10 contiguous amino acids of any one of i) through ii),
b) a nucleic acid sequence encoding a polypeptide as defined in a);
c) a vector comprising the nucleic acid molecule as defined in b),
d) an isolated host cell transformed or transduced with the nucleic acid of b) or the vector of c).

92. An isolated polypeptide for use in a method of treatment or prevention of cardiovascular diseases resulting from hyperglycaemia, said polypeptide comprising an amino acid sequence selected from the group consisting of:
a) the amino acid sequence of SEQ ID NO: 1; and
b) a biologically active sequence variant of a), wherein the variant has at least 70% sequence identity to SEQ ID NO:1,
wherein said polypeptide has a length that is less than 100 amino acids, and
wherein said biological activity is induction of glucose uptake in cells.

93. The polypeptide according to claim 82, wherein said disease is selected from the group consisting of disorders of lipoprotein metabolism and other lipidaemias; abnormal findings from blood; diseases of arteries, arterioles and capillaries; ischaemic and other heart diseases.

94. The polypeptide according to any one of claims 92 to 93, wherein said disease is a disorder of lipoprotein metabolism and other lipidaemias such as a disorder of lipoprotein metabolism and other lipidaemias selected from the group consisting of pure hypercholesterolaemia such as familial hypercholesterolaemia; Fredrickson hyperlipoproteinaemia, type Ila; Hyperbetalipoproteinaemia; Hyperlipidaemia group A; and Low-density-lipoprotein-type [LDL] hyperlipoproteinaemia.

95. The polypeptide according to any one of claims 92 to 94, wherein said disorder of lipoprotein metabolism and other lipidaemias is selected from the group consisting of pure hyperglyceridaemia including endogenous hyperglyceridaemia; Fredrickson hyperlipoproteinaemia, type IV; Hyperlipidaemia, group B; hyperprebetalipoproteinaemia; and very-low-density-lipoprotein-type [VLDL] hyperlipoproteinaemia.

96. The polypeptide according to any one of claims 92 to 95, wherein said disorder of lipoprotein metabolism and other lipidaemias is selected from the group consisting of mixed hyperlipidaemia such as Broad- or floating-betalipoproteinaemia; Fredrickson hyperlipoproteinaemia, type IIb or III; Hyperbetalipoproteinaemia with prebetalipoproteinaemia; hypercholesterolaemia with endogenous hyperglyceridaemia; hyperlipidaemia, group C; tubero-eruptive xanthoma; and Xanthoma tuberosum.

97. The polypeptide according to any one of claims 92 to 96, wherein said disorder of lipoprotein metabolism and other lipidaemias is selected from the group consisting of hyperchylomicronaemia such as Fredrickson hyperlipoproteinaemia, type I or V; hyperlipidaemia, group D; and mixed hyperglyceridaemia.

98. The polypeptide according to any one of claims 92 to 97, wherein said disorder of lipoprotein metabolism and other lipidaemias is selected from the group consisting of other hyperlipidaemia such as familial combined hyperlipidaemia.

99. The polypeptide according to any one of claims 92 to 98, wherein said disorder of lipoprotein metabolism and other lipidaemias is selected from the group consisting of lipoprotein deficiency such as abetalipoproteinaemia, high-density lipoprotein deficiency, hypoalphalipoproteinaemia; hypobetalipoproteinaemia (familial); lecithin cholesterol acyltransferase deficiency and Tangier's disease.

100. The polypeptide according to any one of claims 92 to 99, wherein said diseases of arteries, arterioles and capillaries are selected from the group consisting of atherosclerosis such as arteriolosclerosis; arteriosclerotic vascular disease; atheroma; arterial degeneration; arteriovascular degeneration and vascular degeneration.

101. The polypeptide according to any one of claims 92 to 100, wherein said diseases of arteries, arterioles and capillaries are selected from the group consisting of atherosclerotic heart disease such as coronary (artery) heart disease.

102. The polypeptide according to any one of claims 92 to 101, wherein said ischaemic and other heart diseases are selected from the group consisting of angina pectoris; myocardial infarction; aortic stenosis; and cardiomyopathy in metabolic diseases.

103. The polypeptide according to any one of claims 92 to 102, wherein said cardiovascular disease is characterized by non-normal lipid levels or a lipid containing deposition within body components.

104. The polypeptide according to any one of claims 92 to 103, wherein said cardiovascular disease is selected from the group consisting of: acute coronary syndrome, atherosclerosis, atherosclerotic plaques in blood vessels, valvular stenosis, septic shock, angina pectoris, myocardial infarction, unstable angina pectoris, arterial stenoses, peripheral artery diseases (PAD), carotis stenosis, cerebral arterial stenosis, coronary arterial stenosis, vascular demencia, restenosis, vulnerable plaqueor and amaurosis fugax.


SEQUENCE LISTING (1035 sequences)

SEQ ID NO: 1 - protein, 54 aa, Homo Sapiens
AEYHAKATEHLSTLSEKAKPALEDLRQGLLPVLESFKVSFLSALEEYTKKLNTQ

SEQ ID NO: 2 - protein, 18 aa, Homo Sapiens
AEYHAKATEHLSTLSEKA

SEQ ID NO: 3 - protein, 18 aa, Homo Sapiens
KPALEDLRQGLLPVLESF

SEQ ID NO: 4 - protein, 18 aa, Homo Sapiens
KVSFLSALEEYTKKLNTQ

SEQ ID NO: 5 - protein, 18 aa, Homo Sapiens
HLSTLSEKAKPALEDLRQ

SEQ ID NO: 6 - protein, 18 aa, Homo Sapiens
GLLPVLESFKVSFLSALE

SEQ ID NO: 7 - protein, 27 aa, Homo Sapiens
AEYHAKATEHLSTLSEKAKPALEDLRQ

SEQ ID NO: 8 - protein, 27 aa, Homo Sapiens
GLLPVLESFKVSFLSALEEYTKKLNTQ

SEQ ID NO: 9 - protein, 53 aa, Homo Sapiens
AEYHAKATEHLSTLSEKAKPALEDLRQGLLPVLESFKVSFLSALEEYTKKLNT

SEQ ID NO: 10 - protein, 53 aa, Homo Sapiens
EYHAKATEHLSTLSEKAKPALEDLRQGLLPVLESFKVSFLSALEEYTKKLNTQ

SEQ ID NO: 11 - protein, 52 aa, Homo Sapiens
AEYHAKATEHLSTLSEKAKPALEDLRQGLLPVLESFKVSFLSALEEYTKKLN

SEQ ID NO: 12 - protein, 52 aa, Homo Sapiens
EYHAKATEHLSTLSEKAKPALEDLRQGLLPVLESFKVSFLSALEEYTKKLNT

SEQ ID NO: 13 - protein, 52 aa, Homo Sapiens
YHAKATEHLSTLSEKAKPALEDLRQGLLPVLESFKVSFLSALEEYTKKLNTQ

SEQ ID NO: 14 - protein, 51 aa, Homo Sapiens
AEYHAKATEHLSTLSEKAKPALEDLRQGLLPVLESFKVSFLSALEEYTKKL

SEQ ID NO: 15 - protein, 51 aa, Homo Sapiens
EYHAKATEHLSTLSEKAKPALEDLRQGLLPVLESFKVSFLSALEEYTKKLN

SEQ ID NO: 16 - protein, 51 aa, Homo Sapiens
YHAKATEHLSTLSEKAKPALEDLRQGLLPVLESFKVSFLSALEEYTKKLNT

SEQ ID NO: 17 - protein, 51 aa, Homo Sapiens
HAKATEHLSTLSEKAKPALEDLRQGLLPVLESFKVSFLSALEEYTKKLNTQ

SEQ ID NO: 18 - protein, 50 aa, Homo Sapiens
AEYHAKATEHLSTLSEKAKPALEDLRQGLLPVLESFKVSFLSALEEYTKK

SEQ ID NO: 19 - protein, 50 aa, Homo Sapiens
EYHAKATEHLSTLSEKAKPALEDLRQGLLPVLESFKVSFLSALEEYTKKL

SEQ ID NO: 20 - protein, 50 aa, Homo Sapiens
YHAKATEHLSTLSEKAKPALEDLRQGLLPVLESFKVSFLSALEEYTKKLN

SEQ ID NO: 21 - protein, 50 aa, Homo Sapiens
HAKATEHLSTLSEKAKPALEDLRQGLLPVLESFKVSFLSALEEYTKKLNT

SEQ ID NO: 22 - protein, 50 aa, Homo Sapiens
AKATEHLSTLSEKAKPALEDLRQGLLPVLESFKVSFLSALEEYTKKLNTQ

SEQ ID NO: 23 - protein, 49 aa, Homo Sapiens
AEYHAKATEHLSTLSEKAKPALEDLRQGLLPVLESFKVSFLSALEEYTK

SEQ ID NO: 24 - protein, 49 aa, Homo Sapiens
EYHAKATEHLSTLSEKAKPALEDLRQGLLPVLESFKVSFLSALEEYTKK

SEQ ID NO: 25 - protein, 49 aa, Homo Sapiens
YHAKATEHLSTLSEKAKPALEDLRQGLLPVLESFKVSFLSALEEYTKKL

SEQ ID NO: 26 - protein, 49 aa, Homo Sapiens
HAKATEHLSTLSEKAKPALEDLRQGLLPVLESFKVSFLSALEEYTKKLN

SEQ ID NO: 27 - protein, 49 aa, Homo Sapiens
AKATEHLSTLSEKAKPALEDLRQGLLPVLESFKVSFLSALEEYTKKLNT

SEQ ID NO: 28 - protein, 49 aa, Homo Sapiens
KATEHLSTLSEKAKPALEDLRQGLLPVLESFKVSFLSALEEYTKKLNTQ

SEQ ID NO: 29 - protein, 48 aa, Homo Sapiens
AEYHAKATEHLSTLSEKAKPALEDLRQGLLPVLESFKVSFLSALEEYT

SEQ ID NO: 30 - protein, 48 aa, Homo Sapiens
EYHAKATEHLSTLSEKAKPALEDLRQGLLPVLESFKVSFLSALEEYTK

SEQ ID NO: 31 - protein, 48 aa, Homo Sapiens
YHAKATEHLSTLSEKAKPALEDLRQGLLPVLESFKVSFLSALEEYTKK

SEQ ID NO: 32 - protein, 48 aa, Homo Sapiens
HAKATEHLSTLSEKAKPALEDLRQGLLPVLESFKVSFLSALEEYTKKL

SEQ ID NO: 33 - protein, 48 aa, Homo Sapiens
AKATEHLSTLSEKAKPALEDLRQGLLPVLESFKVSFLSALEEYTKKLN

SEQ ID NO: 34 - protein, 48 aa, Homo Sapiens
KATEHLSTLSEKAKPALEDLRQGLLPVLESFKVSFLSALEEYTKKLNT

SEQ ID NO: 35 - protein, 48 aa, Homo Sapiens
ATEHLSTLSEKAKPALEDLRQGLLPVLESFKVSFLSALEEYTKKLNTQ

SEQ ID NO: 36 - protein, 47 aa, Homo Sapiens
AEYHAKATEHLSTLSEKAKPALEDLRQGLLPVLESFKVSFLSALEEY

SEQ ID NO: 37 - protein, 47 aa, Homo Sapiens
EYHAKATEHLSTLSEKAKPALEDLRQGLLPVLESFKVSFLSALEEYT

SEQ ID NO: 38 - protein, 47 aa, Homo Sapiens
YHAKATEHLSTLSEKAKPALEDLRQGLLPVLESFKVSFLSALEEYTK

SEQ ID NO: 39 - protein, 47 aa, Homo Sapiens
HAKATEHLSTLSEKAKPALEDLRQGLLPVLESFKVSFLSALEEYTKK

SEQ ID NO: 40 - protein, 47 aa, Homo Sapiens
AKATEHLSTLSEKAKPALEDLRQGLLPVLESFKVSFLSALEEYTKKL

SEQ ID NO: 41 - protein, 47 aa, Homo Sapiens
KATEHLSTLSEKAKPALEDLRQGLLPVLESFKVSFLSALEEYTKKLN

SEQ ID NO: 42 - protein, 47 aa, Homo Sapiens
ATEHLSTLSEKAKPALEDLRQGLLPVLESFKVSFLSALEEYTKKLNT

SEQ ID NO: 43 - protein, 47 aa, Homo Sapiens
TEHLSTLSEKAKPALEDLRQGLLPVLESFKVSFLSALEEYTKKLNTQ

SEQ ID NO: 44 - protein, 46 aa, Homo Sapiens
AEYHAKATEHLSTLSEKAKPALEDLRQGLLPVLESFKVSFLSALEE

SEQ ID NO: 45 - protein, 46 aa, Homo Sapiens
EYHAKATEHLSTLSEKAKPALEDLRQGLLPVLESFKVSFLSALEEY

SEQ ID NO: 46 - protein, 46 aa, Homo Sapiens
YHAKATEHLSTLSEKAKPALEDLRQGLLPVLESFKVSFLSALEEYT

SEQ ID NO: 47 - protein, 46 aa, Homo Sapiens
HAKATEHLSTLSEKAKPALEDLRQGLLPVLESFKVSFLSALEEYTK

SEQ ID NO: 48 - protein, 46 aa, Homo Sapiens
AKATEHLSTLSEKAKPALEDLRQGLLPVLESFKVSFLSALEEYTKK

SEQ ID NO: 49 - protein, 46 aa, Homo Sapiens
KATEHLSTLSEKAKPALEDLRQGLLPVLESFKVSFLSALEEYTKKL

SEQ ID NO: 50 - protein, 46 aa, Homo Sapiens
ATEHLSTLSEKAKPALEDLRQGLLPVLESFKVSFLSALEEYTKKLN

SEQ ID NO: 51 - protein, 46 aa, Homo Sapiens
TEHLSTLSEKAKPALEDLRQGLLPVLESFKVSFLSALEEYTKKLNT

SEQ ID NO: 52 - protein, 46 aa, Homo Sapiens
EHLSTLSEKAKPALEDLRQGLLPVLESFKVSFLSALEEYTKKLNTQ

SEQ ID NO: 53 - protein, 45 aa, Homo Sapiens
AEYHAKATEHLSTLSEKAKPALEDLRQGLLPVLESFKVSFLSALE

SEQ ID NO: 54 - protein, 45 aa, Homo Sapiens
EYHAKATEHLSTLSEKAKPALEDLRQGLLPVLESFKVSFLSALEE

SEQ ID NO: 55 - protein, 45 aa, Homo Sapiens
YHAKATEHLSTLSEKAKPALEDLRQGLLPVLESFKVSFLSALEEY

SEQ ID NO: 56 - protein, 45 aa, Homo Sapiens
HAKATEHLSTLSEKAKPALEDLRQGLLPVLESFKVSFLSALEEYT

SEQ ID NO: 57 - protein, 45 aa, Homo Sapiens
AKATEHLSTLSEKAKPALEDLRQGLLPVLESFKVSFLSALEEYTK

SEQ ID NO: 58 - protein, 10 aa, Homo Sapiens
EEYTKKLNTQ

SEQ ID NO: 59 - protein, 10 aa, Homo Sapiens
ALEEYTKKLN

SEQ ID NO: 60 - protein, 45 aa, Homo Sapiens
KATEHLSTLSEKAKPALEDLRQGLLPVLESFKVSFLSALEEYTKK

SEQ ID NO: 61 - protein, 45 aa, Homo Sapiens
ATEHLSTLSEKAKPALEDLRQGLLPVLESFKVSFLSALEEYTKKL

SEQ ID NO: 62 - protein, 45 aa, Homo Sapiens
TEHLSTLSEKAKPALEDLRQGLLPVLESFKVSFLSALEEYTKKLN

SEQ ID NO: 63 - protein, 45 aa, Homo Sapiens
EHLSTLSEKAKPALEDLRQGLLPVLESFKVSFLSALEEYTKKLNT

SEQ ID NO: 64 - protein, 45 aa, Homo Sapiens
HLSTLSEKAKPALEDLRQGLLPVLESFKVSFLSALEEYTKKLNTQ

SEQ ID NO: 65 - protein, 44 aa, Homo Sapiens
AEYHAKATEHLSTLSEKAKPALEDLRQGLLPVLESFKVSFLSAL

SEQ ID NO: 66 - protein, 44 aa, Homo Sapiens
EYHAKATEHLSTLSEKAKPALEDLRQGLLPVLESFKVSFLSALE

SEQ ID NO: 67 - protein, 44 aa, Homo Sapiens
YHAKATEHLSTLSEKAKPALEDLRQGLLPVLESFKVSFLSALEE

SEQ ID NO: 68 - protein, 44 aa, Homo Sapiens
HAKATEHLSTLSEKAKPALEDLRQGLLPVLESFKVSFLSALEEY

SEQ ID NO: 69 - protein, 44 aa, Homo Sapiens
AKATEHLSTLSEKAKPALEDLRQGLLPVLESFKVSFLSALEEYT

SEQ ID NO: 70 - protein, 44 aa, Homo Sapiens
KATEHLSTLSEKAKPALEDLRQGLLPVLESFKVSFLSALEEYTK

SEQ ID NO: 71 - protein, 44 aa, Homo Sapiens
ATEHLSTLSEKAKPALEDLRQGLLPVLESFKVSFLSALEEYTKK

SEQ ID NO: 72 - protein, 44 aa, Homo Sapiens
TEHLSTLSEKAKPALEDLRQGLLPVLESFKVSFLSALEEYTKKL

SEQ ID NO: 73 - protein, 44 aa, Homo Sapiens
EHLSTLSEKAKPALEDLRQGLLPVLESFKVSFLSALEEYTKKLN

SEQ ID NO: 74 - protein, 44 aa, Homo Sapiens
HLSTLSEKAKPALEDLRQGLLPVLESFKVSFLSALEEYTKKLNT

SEQ ID NO: 75 - protein, 44 aa, Homo Sapiens
LSTLSEKAKPALEDLRQGLLPVLESFKVSFLSALEEYTKKLNTQ

SEQ ID NO: 76 - protein, 43 aa, Homo Sapiens
AEYHAKATEHLSTLSEKAKPALEDLRQGLLPVLESFKVSFLSA

SEQ ID NO: 77 - protein, 43 aa, Homo Sapiens
EYHAKATEHLSTLSEKAKPALEDLRQGLLPVLESFKVSFLSAL

SEQ ID NO: 78 - protein, 43 aa, Homo Sapiens
YHAKATEHLSTLSEKAKPALEDLRQGLLPVLESFKVSFLSALE

SEQ ID NO: 79 - protein, 43 aa, Homo Sapiens
HAKATEHLSTLSEKAKPALEDLRQGLLPVLESFKVSFLSALEE

SEQ ID NO: 80 - protein, 43 aa, Homo Sapiens
AKATEHLSTLSEKAKPALEDLRQGLLPVLESFKVSFLSALEEY

SEQ ID NO: 81 - protein, 43 aa, Homo Sapiens
KATEHLSTLSEKAKPALEDLRQGLLPVLESFKVSFLSALEEYT

SEQ ID NO: 82 - protein, 43 aa, Homo Sapiens
ATEHLSTLSEKAKPALEDLRQGLLPVLESFKVSFLSALEEYTK

SEQ ID NO: 83 - protein, 43 aa, Homo Sapiens
TEHLSTLSEKAKPALEDLRQGLLPVLESFKVSFLSALEEYTKK

SEQ ID NO: 84 - protein, 43 aa, Homo Sapiens
EHLSTLSEKAKPALEDLRQGLLPVLESFKVSFLSALEEYTKKL

SEQ ID NO: 85 - protein, 43 aa, Homo Sapiens
HLSTLSEKAKPALEDLRQGLLPVLESFKVSFLSALEEYTKKLN

SEQ ID NO: 86 - protein, 43 aa, Homo Sapiens
LSTLSEKAKPALEDLRQGLLPVLESFKVSFLSALEEYTKKLNT

SEQ ID NO: 87 - protein, 43 aa, Homo Sapiens
STLSEKAKPALEDLRQGLLPVLESFKVSFLSALEEYTKKLNTQ

SEQ ID NO: 88 - protein, 42 aa, Homo Sapiens
AEYHAKATEHLSTLSEKAKPALEDLRQGLLPVLESFKVSFLS

SEQ ID NO: 89 - protein, 42 aa, Homo Sapiens
EYHAKATEHLSTLSEKAKPALEDLRQGLLPVLESFKVSFLSA

SEQ ID NO: 90 - protein, 42 aa, Homo Sapiens
YHAKATEHLSTLSEKAKPALEDLRQGLLPVLESFKVSFLSAL

SEQ ID NO: 91 - protein, 42 aa, Homo Sapiens
HAKATEHLSTLSEKAKPALEDLRQGLLPVLESFKVSFLSALE

SEQ ID NO: 92 - protein, 42 aa, Homo Sapiens
AKATEHLSTLSEKAKPALEDLRQGLLPVLESFKVSFLSALEE

SEQ ID NO: 93 - protein, 42 aa, Homo Sapiens
KATEHLSTLSEKAKPALEDLRQGLLPVLESFKVSFLSALEEY

SEQ ID NO: 94 - protein, 42 aa, Homo Sapiens
ATEHLSTLSEKAKPALEDLRQGLLPVLESFKVSFLSALEEYT

SEQ ID NO: 95 - protein, 42 aa, Homo Sapiens
TEHLSTLSEKAKPALEDLRQGLLPVLESFKVSFLSALEEYTK

SEQ ID NO: 96 - protein, 42 aa, Homo Sapiens
EHLSTLSEKAKPALEDLRQGLLPVLESFKVSFLSALEEYTKK

SEQ ID NO: 97 - protein, 42 aa, Homo Sapiens
HLSTLSEKAKPALEDLRQGLLPVLESFKVSFLSALEEYTKKL

SEQ ID NO: 98 - protein, 42 aa, Homo Sapiens
LSTLSEKAKPALEDLRQGLLPVLESFKVSFLSALEEYTKKLN

SEQ ID NO: 99 - protein, 42 aa, Homo Sapiens
STLSEKAKPALEDLRQGLLPVLESFKVSFLSALEEYTKKLNT

SEQ ID NO: 100 - protein, 42 aa, Homo Sapiens
TLSEKAKPALEDLRQGLLPVLESFKVSFLSALEEYTKKLNTQ

SEQ ID NO: 101 - protein, 41 aa, Homo Sapiens
AEYHAKATEHLSTLSEKAKPALEDLRQGLLPVLESFKVSFL

SEQ ID NO: 102 - protein, 41 aa, Homo Sapiens
EYHAKATEHLSTLSEKAKPALEDLRQGLLPVLESFKVSFLS

SEQ ID NO: 103 - protein, 41 aa, Homo Sapiens
YHAKATEHLSTLSEKAKPALEDLRQGLLPVLESFKVSFLSA

SEQ ID NO: 104 - protein, 41 aa, Homo Sapiens
HAKATEHLSTLSEKAKPALEDLRQGLLPVLESFKVSFLSAL

SEQ ID NO: 105 - protein, 41 aa, Homo Sapiens
AKATEHLSTLSEKAKPALEDLRQGLLPVLESFKVSFLSALE

SEQ ID NO: 106 - protein, 41 aa, Homo Sapiens
KATEHLSTLSEKAKPALEDLRQGLLPVLESFKVSFLSALEE

SEQ ID NO: 107 - protein, 41 aa, Homo Sapiens
ATEHLSTLSEKAKPALEDLRQGLLPVLESFKVSFLSALEEY

SEQ ID NO: 108 - protein, 41 aa, Homo Sapiens
TEHLSTLSEKAKPALEDLRQGLLPVLESFKVSFLSALEEYT

SEQ ID NO: 109 - protein, 41 aa, Homo Sapiens
EHLSTLSEKAKPALEDLRQGLLPVLESFKVSFLSALEEYTK

SEQ ID NO: 110 - protein, 41 aa, Homo Sapiens
HLSTLSEKAKPALEDLRQGLLPVLESFKVSFLSALEEYTKK

SEQ ID NO: 111 - protein, 41 aa, Homo Sapiens
LSTLSEKAKPALEDLRQGLLPVLESFKVSFLSALEEYTKKL

SEQ ID NO: 112 - protein, 41 aa, Homo Sapiens
STLSEKAKPALEDLRQGLLPVLESFKVSFLSALEEYTKKLN

SEQ ID NO: 113 - protein, 41 aa, Homo Sapiens
TLSEKAKPALEDLRQGLLPVLESFKVSFLSALEEYTKKLNT

SEQ ID NO: 114 - protein, 41 aa, Homo Sapiens
LSEKAKPALEDLRQGLLPVLESFKVSFLSALEEYTKKLNTQ

SEQ ID NO: 115 - protein, 40 aa, Homo Sapiens
AEYHAKATEHLSTLSEKAKPALEDLRQGLLPVLESFKVSF

SEQ ID NO: 116 - protein, 40 aa, Homo Sapiens
EYHAKATEHLSTLSEKAKPALEDLRQGLLPVLESFKVSFL

SEQ ID NO: 117 - protein, 40 aa, Homo Sapiens
YHAKATEHLSTLSEKAKPALEDLRQGLLPVLESFKVSFLS

SEQ ID NO: 118 - protein, 40 aa, Homo Sapiens
HAKATEHLSTLSEKAKPALEDLRQGLLPVLESFKVSFLSA

SEQ ID NO: 119 - protein, 40 aa, Homo Sapiens
AKATEHLSTLSEKAKPALEDLRQGLLPVLESFKVSFLSAL

SEQ ID NO: 120 - protein, 40 aa, Homo Sapiens
KATEHLSTLSEKAKPALEDLRQGLLPVLESFKVSFLSALE

SEQ ID NO: 121 - protein, 40 aa, Homo Sapiens
ATEHLSTLSEKAKPALEDLRQGLLPVLESFKVSFLSALEE

SEQ ID NO: 122 - protein, 40 aa, Homo Sapiens
TEHLSTLSEKAKPALEDLRQGLLPVLESFKVSFLSALEEY

SEQ ID NO: 123 - protein, 40 aa, Homo Sapiens
EHLSTLSEKAKPALEDLRQGLLPVLESFKVSFLSALEEYT

SEQ ID NO: 124 - protein, 40 aa, Homo Sapiens
HLSTLSEKAKPALEDLRQGLLPVLESFKVSFLSALEEYTK

SEQ ID NO: 125 - protein, 40 aa, Homo Sapiens
LSTLSEKAKPALEDLRQGLLPVLESFKVSFLSALEEYTKK

SEQ ID NO: 126 - protein, 40 aa, Homo Sapiens
STLSEKAKPALEDLRQGLLPVLESFKVSFLSALEEYTKKL

SEQ ID NO: 127 - protein, 40 aa, Homo Sapiens
TLSEKAKPALEDLRQGLLPVLESFKVSFLSALEEYTKKLN

SEQ ID NO: 128 - protein, 40 aa, Homo Sapiens
LSEKAKPALEDLRQGLLPVLESFKVSFLSALEEYTKKLNT

SEQ ID NO: 129 - protein, 40 aa, Homo Sapiens
SEKAKPALEDLRQGLLPVLESFKVSFLSALEEYTKKLNTQ

SEQ ID NO: 130 - protein, 39 aa, Homo Sapiens
AEYHAKATEHLSTLSEKAKPALEDLRQGLLPVLESFKVS

SEQ ID NO: 131 - protein, 39 aa, Homo Sapiens
EYHAKATEHLSTLSEKAKPALEDLRQGLLPVLESFKVSF

SEQ ID NO: 132 - protein, 39 aa, Homo Sapiens
YHAKATEHLSTLSEKAKPALEDLRQGLLPVLESFKVSFL

SEQ ID NO: 133 - protein, 39 aa, Homo Sapiens
HAKATEHLSTLSEKAKPALEDLRQGLLPVLESFKVSFLS

SEQ ID NO: 134 - protein, 39 aa, Homo Sapiens
AKATEHLSTLSEKAKPALEDLRQGLLPVLESFKVSFLSA

SEQ ID NO: 135 - protein, 39 aa, Homo Sapiens
KATEHLSTLSEKAKPALEDLRQGLLPVLESFKVSFLSAL

SEQ ID NO: 136 - protein, 39 aa, Homo Sapiens
ATEHLSTLSEKAKPALEDLRQGLLPVLESFKVSFLSALE

SEQ ID NO: 137 - protein, 39 aa, Homo Sapiens
TEHLSTLSEKAKPALEDLRQGLLPVLESFKVSFLSALEE

SEQ ID NO: 138 - protein, 39 aa, Homo Sapiens
EHLSTLSEKAKPALEDLRQGLLPVLESFKVSFLSALEEY

SEQ ID NO: 139 - protein, 39 aa, Homo Sapiens
HLSTLSEKAKPALEDLRQGLLPVLESFKVSFLSALEEYT

SEQ ID NO: 140 - protein, 39 aa, Homo Sapiens
LSTLSEKAKPALEDLRQGLLPVLESFKVSFLSALEEYTK

SEQ ID NO: 141 - protein, 39 aa, Homo Sapiens
STLSEKAKPALEDLRQGLLPVLESFKVSFLSALEEYTKK

SEQ ID NO: 142 - protein, 39 aa, Homo Sapiens
TLSEKAKPALEDLRQGLLPVLESFKVSFLSALEEYTKKL

SEQ ID NO: 143 - protein, 39 aa, Homo Sapiens
LSEKAKPALEDLRQGLLPVLESFKVSFLSALEEYTKKLN

SEQ ID NO: 144 - protein, 39 aa, Homo Sapiens
SEKAKPALEDLRQGLLPVLESFKVSFLSALEEYTKKLNT

SEQ ID NO: 145 - protein, 39 aa, Homo Sapiens
EKAKPALEDLRQGLLPVLESFKVSFLSALEEYTKKLNTQ

SEQ ID NO: 146 - protein, 38 aa, Homo Sapiens
AEYHAKATEHLSTLSEKAKPALEDLRQGLLPVLESFKV

SEQ ID NO: 147 - protein, 38 aa, Homo Sapiens
EYHAKATEHLSTLSEKAKPALEDLRQGLLPVLESFKVS

SEQ ID NO: 148 - protein, 38 aa, Homo Sapiens
YHAKATEHLSTLSEKAKPALEDLRQGLLPVLESFKVSF

SEQ ID NO: 149 - protein, 38 aa, Homo Sapiens
HAKATEHLSTLSEKAKPALEDLRQGLLPVLESFKVSFL

SEQ ID NO: 150 - protein, 38 aa, Homo Sapiens
AKATEHLSTLSEKAKPALEDLRQGLLPVLESFKVSFLS

SEQ ID NO: 151 - protein, 38 aa, Homo Sapiens
KATEHLSTLSEKAKPALEDLRQGLLPVLESFKVSFLSA

SEQ ID NO: 152 - protein, 38 aa, Homo Sapiens
ATEHLSTLSEKAKPALEDLRQGLLPVLESFKVSFLSAL

SEQ ID NO: 153 - protein, 38 aa, Homo Sapiens
TEHLSTLSEKAKPALEDLRQGLLPVLESFKVSFLSALE

SEQ ID NO: 154 - protein, 38 aa, Homo Sapiens
EHLSTLSEKAKPALEDLRQGLLPVLESFKVSFLSALEE

SEQ ID NO: 155 - protein, 38 aa, Homo Sapiens
HLSTLSEKAKPALEDLRQGLLPVLESFKVSFLSALEEY

SEQ ID NO: 156 - protein, 38 aa, Homo Sapiens
LSTLSEKAKPALEDLRQGLLPVLESFKVSFLSALEEYT

SEQ ID NO: 157 - protein, 38 aa, Homo Sapiens
STLSEKAKPALEDLRQGLLPVLESFKVSFLSALEEYTK

SEQ ID NO: 158 - protein, 38 aa, Homo Sapiens
TLSEKAKPALEDLRQGLLPVLESFKVSFLSALEEYTKK

SEQ ID NO: 159 - protein, 38 aa, Homo Sapiens
LSEKAKPALEDLRQGLLPVLESFKVSFLSALEEYTKKL

SEQ ID NO: 160 - protein, 38 aa, Homo Sapiens
SEKAKPALEDLRQGLLPVLESFKVSFLSALEEYTKKLN

SEQ ID NO: 161 - protein, 38 aa, Homo Sapiens
EKAKPALEDLRQGLLPVLESFKVSFLSALEEYTKKLNT

SEQ ID NO: 162 - protein, 38 aa, Homo Sapiens
KAKPALEDLRQGLLPVLESFKVSFLSALEEYTKKLNTQ

SEQ ID NO: 163 - protein, 37 aa, Homo Sapiens
AEYHAKATEHLSTLSEKAKPALEDLRQGLLPVLESFK

SEQ ID NO: 164 - protein, 37 aa, Homo Sapiens
EYHAKATEHLSTLSEKAKPALEDLRQGLLPVLESFKV

SEQ ID NO: 165 - protein, 37 aa, Homo Sapiens
YHAKATEHLSTLSEKAKPALEDLRQGLLPVLESFKVS

SEQ ID NO: 166 - protein, 37 aa, Homo Sapiens
HAKATEHLSTLSEKAKPALEDLRQGLLPVLESFKVSF

SEQ ID NO: 167 - protein, 37 aa, Homo Sapiens
AKATEHLSTLSEKAKPALEDLRQGLLPVLESFKVSFL

SEQ ID NO: 168 - protein, 37 aa, Homo Sapiens
KATEHLSTLSEKAKPALEDLRQGLLPVLESFKVSFLS

SEQ ID NO: 169 - protein, 37 aa, Homo Sapiens
ATEHLSTLSEKAKPALEDLRQGLLPVLESFKVSFLSA

SEQ ID NO: 170 - protein, 37 aa, Homo Sapiens
TEHLSTLSEKAKPALEDLRQGLLPVLESFKVSFLSAL

SEQ ID NO: 171 - protein, 37 aa, Homo Sapiens
EHLSTLSEKAKPALEDLRQGLLPVLESFKVSFLSALE

SEQ ID NO: 172 - protein, 37 aa, Homo Sapiens
HLSTLSEKAKPALEDLRQGLLPVLESFKVSFLSALEE

SEQ ID NO: 173 - protein, 37 aa, Homo Sapiens
LSTLSEKAKPALEDLRQGLLPVLESFKVSFLSALEEY

SEQ ID NO: 174 - protein, 37 aa, Homo Sapiens
STLSEKAKPALEDLRQGLLPVLESFKVSFLSALEEYT

SEQ ID NO: 175 - protein, 37 aa, Homo Sapiens
TLSEKAKPALEDLRQGLLPVLESFKVSFLSALEEYTK

SEQ ID NO: 176 - protein, 37 aa, Homo Sapiens
LSEKAKPALEDLRQGLLPVLESFKVSFLSALEEYTKK

SEQ ID NO: 177 - protein, 37 aa, Homo Sapiens
SEKAKPALEDLRQGLLPVLESFKVSFLSALEEYTKKL

SEQ ID NO: 178 - protein, 37 aa, Homo Sapiens
EKAKPALEDLRQGLLPVLESFKVSFLSALEEYTKKLN

SEQ ID NO: 179 - protein, 37 aa, Homo Sapiens
KAKPALEDLRQGLLPVLESFKVSFLSALEEYTKKLNT

SEQ ID NO: 180 - protein, 37 aa, Homo Sapiens
AKPALEDLRQGLLPVLESFKVSFLSALEEYTKKLNTQ

SEQ ID NO: 181 - protein, 36 aa, Homo Sapiens
AEYHAKATEHLSTLSEKAKPALEDLRQGLLPVLESF

SEQ ID NO: 182 - protein, 36 aa, Homo Sapiens
EYHAKATEHLSTLSEKAKPALEDLRQGLLPVLESFK

SEQ ID NO: 183 - protein, 36 aa, Homo Sapiens
YHAKATEHLSTLSEKAKPALEDLRQGLLPVLESFKV

SEQ ID NO: 184 - protein, 36 aa, Homo Sapiens
HAKATEHLSTLSEKAKPALEDLRQGLLPVLESFKVS

SEQ ID NO: 185 - protein, 36 aa, Homo Sapiens
AKATEHLSTLSEKAKPALEDLRQGLLPVLESFKVSF

SEQ ID NO: 186 - protein, 36 aa, Homo Sapiens
KATEHLSTLSEKAKPALEDLRQGLLPVLESFKVSFL

SEQ ID NO: 187 - protein, 36 aa, Homo Sapiens
ATEHLSTLSEKAKPALEDLRQGLLPVLESFKVSFLS

SEQ ID NO: 188 - protein, 36 aa, Homo Sapiens
TEHLSTLSEKAKPALEDLRQGLLPVLESFKVSFLSA

SEQ ID NO: 189 - protein, 36 aa, Homo Sapiens
EHLSTLSEKAKPALEDLRQGLLPVLESFKVSFLSAL

SEQ ID NO: 190 - protein, 36 aa, Homo Sapiens
HLSTLSEKAKPALEDLRQGLLPVLESFKVSFLSALE

SEQ ID NO: 191 - protein, 36 aa, Homo Sapiens
LSTLSEKAKPALEDLRQGLLPVLESFKVSFLSALEE

SEQ ID NO: 192 - protein, 36 aa, Homo Sapiens
STLSEKAKPALEDLRQGLLPVLESFKVSFLSALEEY

SEQ ID NO: 193 - protein, 36 aa, Homo Sapiens
TLSEKAKPALEDLRQGLLPVLESFKVSFLSALEEYT

SEQ ID NO: 194 - protein, 36 aa, Homo Sapiens
LSEKAKPALEDLRQGLLPVLESFKVSFLSALEEYTK

SEQ ID NO: 195 - protein, 36 aa, Homo Sapiens
SEKAKPALEDLRQGLLPVLESFKVSFLSALEEYTKK

SEQ ID NO: 196 - protein, 36 aa, Homo Sapiens
EKAKPALEDLRQGLLPVLESFKVSFLSALEEYTKKL

SEQ ID NO: 197 - protein, 36 aa, Homo Sapiens
KAKPALEDLRQGLLPVLESFKVSFLSALEEYTKKLN

SEQ ID NO: 198 - protein, 36 aa, Homo Sapiens
AKPALEDLRQGLLPVLESFKVSFLSALEEYTKKLNT

SEQ ID NO: 199 - protein, 36 aa, Homo Sapiens
KPALEDLRQGLLPVLESFKVSFLSALEEYTKKLNTQ

SEQ ID NO: 200 - protein, 35 aa, Homo Sapiens
AEYHAKATEHLSTLSEKAKPALEDLRQGLLPVLES

SEQ ID NO: 201 - protein, 35 aa, Homo Sapiens
EYHAKATEHLSTLSEKAKPALEDLRQGLLPVLESF

SEQ ID NO: 202 - protein, 35 aa, Homo Sapiens
YHAKATEHLSTLSEKAKPALEDLRQGLLPVLESFK

SEQ ID NO: 203 - protein, 35 aa, Homo Sapiens
HAKATEHLSTLSEKAKPALEDLRQGLLPVLESFKV

SEQ ID NO: 204 - protein, 35 aa, Homo Sapiens
AKATEHLSTLSEKAKPALEDLRQGLLPVLESFKVS

SEQ ID NO: 205 - protein, 35 aa, Homo Sapiens
KATEHLSTLSEKAKPALEDLRQGLLPVLESFKVSF

SEQ ID NO: 206 - protein, 35 aa, Homo Sapiens
ATEHLSTLSEKAKPALEDLRQGLLPVLESFKVSFL

SEQ ID NO: 207 - protein, 35 aa, Homo Sapiens
TEHLSTLSEKAKPALEDLRQGLLPVLESFKVSFLS

SEQ ID NO: 208 - protein, 35 aa, Homo Sapiens
EHLSTLSEKAKPALEDLRQGLLPVLESFKVSFLSA

SEQ ID NO: 209 - protein, 35 aa, Homo Sapiens
HLSTLSEKAKPALEDLRQGLLPVLESFKVSFLSAL

SEQ ID NO: 210 - protein, 35 aa, Homo Sapiens
LSTLSEKAKPALEDLRQGLLPVLESFKVSFLSALE

SEQ ID NO: 211 - protein, 35 aa, Homo Sapiens
STLSEKAKPALEDLRQGLLPVLESFKVSFLSALEE

SEQ ID NO: 212 - protein, 35 aa, Homo Sapiens
TLSEKAKPALEDLRQGLLPVLESFKVSFLSALEEY

SEQ ID NO: 213 - protein, 35 aa, Homo Sapiens
LSEKAKPALEDLRQGLLPVLESFKVSFLSALEEYT

SEQ ID NO: 214 - protein, 35 aa, Homo Sapiens
SEKAKPALEDLRQGLLPVLESFKVSFLSALEEYTK

SEQ ID NO: 215 - protein, 35 aa, Homo Sapiens
EKAKPALEDLRQGLLPVLESFKVSFLSALEEYTKK

SEQ ID NO: 216 - protein, 35 aa, Homo Sapiens
KAKPALEDLRQGLLPVLESFKVSFLSALEEYTKKL

SEQ ID NO: 217 - protein, 35 aa, Homo Sapiens
AKPALEDLRQGLLPVLESFKVSFLSALEEYTKKLN

SEQ ID NO: 218 - protein, 35 aa, Homo Sapiens
KPALEDLRQGLLPVLESFKVSFLSALEEYTKKLNT

SEQ ID NO: 219 - protein, 35 aa, Homo Sapiens
PALEDLRQGLLPVLESFKVSFLSALEEYTKKLNTQ

SEQ ID NO: 220 - protein, 34 aa, Homo Sapiens
AEYHAKATEHLSTLSEKAKPALEDLRQGLLPVLE

SEQ ID NO: 221 - protein, 34 aa, Homo Sapiens
EYHAKATEHLSTLSEKAKPALEDLRQGLLPVLES

SEQ ID NO: 222 - protein, 34 aa, Homo Sapiens
YHAKATEHLSTLSEKAKPALEDLRQGLLPVLESF

SEQ ID NO: 223 - protein, 34 aa, Homo Sapiens
HAKATEHLSTLSEKAKPALEDLRQGLLPVLESFK

SEQ ID NO: 224 - protein, 34 aa, Homo Sapiens
AKATEHLSTLSEKAKPALEDLRQGLLPVLESFKV

SEQ ID NO: 225 - protein, 34 aa, Homo Sapiens
KATEHLSTLSEKAKPALEDLRQGLLPVLESFKVS

SEQ ID NO: 226 - protein, 34 aa, Homo Sapiens
ATEHLSTLSEKAKPALEDLRQGLLPVLESFKVSF

SEQ ID NO: 227 - protein, 34 aa, Homo Sapiens
TEHLSTLSEKAKPALEDLRQGLLPVLESFKVSFL

SEQ ID NO: 228 - protein, 34 aa, Homo Sapiens
EHLSTLSEKAKPALEDLRQGLLPVLESFKVSFLS

SEQ ID NO: 229 - protein, 34 aa, Homo Sapiens
HLSTLSEKAKPALEDLRQGLLPVLESFKVSFLSA

SEQ ID NO: 230 - protein, 34 aa, Homo Sapiens
LSTLSEKAKPALEDLRQGLLPVLESFKVSFLSAL

SEQ ID NO: 231 - protein, 34 aa, Homo Sapiens
STLSEKAKPALEDLRQGLLPVLESFKVSFLSALE

SEQ ID NO: 232 - protein, 34 aa, Homo Sapiens
TLSEKAKPALEDLRQGLLPVLESFKVSFLSALEE

SEQ ID NO: 233 - protein, 34 aa, Homo Sapiens
LSEKAKPALEDLRQGLLPVLESFKVSFLSALEEY

SEQ ID NO: 234 - protein, 34 aa, Homo Sapiens
SEKAKPALEDLRQGLLPVLESFKVSFLSALEEYT

SEQ ID NO: 235 - protein, 34 aa, Homo Sapiens
EKAKPALEDLRQGLLPVLESFKVSFLSALEEYTK

SEQ ID NO: 236 - protein, 34 aa, Homo Sapiens
KAKPALEDLRQGLLPVLESFKVSFLSALEEYTKK

SEQ ID NO: 237 - protein, 34 aa, Homo Sapiens
AKPALEDLRQGLLPVLESFKVSFLSALEEYTKKL

SEQ ID NO: 238 - protein, 34 aa, Homo Sapiens
KPALEDLRQGLLPVLESFKVSFLSALEEYTKKLN

SEQ ID NO: 239 - protein, 34 aa, Homo Sapiens
PALEDLRQGLLPVLESFKVSFLSALEEYTKKLNT

SEQ ID NO: 240 - protein, 34 aa, Homo Sapiens
ALEDLRQGLLPVLESFKVSFLSALEEYTKKLNTQ

SEQ ID NO: 241 - protein, 33 aa, Homo Sapiens
AEYHAKATEHLSTLSEKAKPALEDLRQGLLPVL

SEQ ID NO: 242 - protein, 33 aa, Homo Sapiens
EYHAKATEHLSTLSEKAKPALEDLRQGLLPVLE

SEQ ID NO: 243 - protein, 33 aa, Homo Sapiens
YHAKATEHLSTLSEKAKPALEDLRQGLLPVLES

SEQ ID NO: 244 - protein, 33 aa, Homo Sapiens
HAKATEHLSTLSEKAKPALEDLRQGLLPVLESF

SEQ ID NO: 245 - protein, 33 aa, Homo Sapiens
AKATEHLSTLSEKAKPALEDLRQGLLPVLESFK

SEQ ID NO: 246 - protein, 33 aa, Homo Sapiens
KATEHLSTLSEKAKPALEDLRQGLLPVLESFKV

SEQ ID NO: 247 - protein, 33 aa, Homo Sapiens
ATEHLSTLSEKAKPALEDLRQGLLPVLESFKVS

SEQ ID NO: 248 - protein, 33 aa, Homo Sapiens
TEHLSTLSEKAKPALEDLRQGLLPVLESFKVSF

SEQ ID NO: 249 - protein, 33 aa, Homo Sapiens
EHLSTLSEKAKPALEDLRQGLLPVLESFKVSFL

SEQ ID NO: 250 - protein, 33 aa, Homo Sapiens
HLSTLSEKAKPALEDLRQGLLPVLESFKVSFLS

SEQ ID NO: 251 - protein, 33 aa, Homo Sapiens
LSTLSEKAKPALEDLRQGLLPVLESFKVSFLSA

SEQ ID NO: 252 - protein, 33 aa, Homo Sapiens
STLSEKAKPALEDLRQGLLPVLESFKVSFLSAL

SEQ ID NO: 253 - protein, 33 aa, Homo Sapiens
TLSEKAKPALEDLRQGLLPVLESFKVSFLSALE

SEQ ID NO: 254 - protein, 33 aa, Homo Sapiens
LSEKAKPALEDLRQGLLPVLESFKVSFLSALEE

SEQ ID NO: 255 - protein, 33 aa, Homo Sapiens
SEKAKPALEDLRQGLLPVLESFKVSFLSALEEY

SEQ ID NO: 256 - protein, 33 aa, Homo Sapiens
EKAKPALEDLRQGLLPVLESFKVSFLSALEEYT

SEQ ID NO: 257 - protein, 33 aa, Homo Sapiens
KAKPALEDLRQGLLPVLESFKVSFLSALEEYTK

SEQ ID NO: 258 - protein, 33 aa, Homo Sapiens
AKPALEDLRQGLLPVLESFKVSFLSALEEYTKK

SEQ ID NO: 259 - protein, 33 aa, Homo Sapiens
KPALEDLRQGLLPVLESFKVSFLSALEEYTKKL

SEQ ID NO: 260 - protein, 33 aa, Homo Sapiens
PALEDLRQGLLPVLESFKVSFLSALEEYTKKLN

SEQ ID NO: 261 - protein, 33 aa, Homo Sapiens
ALEDLRQGLLPVLESFKVSFLSALEEYTKKLNT

SEQ ID NO: 262 - protein, 33 aa, Homo Sapiens
LEDLRQGLLPVLESFKVSFLSALEEYTKKLNTQ

SEQ ID NO: 263 - protein, 32 aa, Homo Sapiens
AEYHAKATEHLSTLSEKAKPALEDLRQGLLPV

SEQ ID NO: 264 - protein, 32 aa, Homo Sapiens
EYHAKATEHLSTLSEKAKPALEDLRQGLLPVL

SEQ ID NO: 265 - protein, 32 aa, Homo Sapiens
YHAKATEHLSTLSEKAKPALEDLRQGLLPVLE

SEQ ID NO: 266 - protein, 32 aa, Homo Sapiens
HAKATEHLSTLSEKAKPALEDLRQGLLPVLES

SEQ ID NO: 267 - protein, 32 aa, Homo Sapiens
AKATEHLSTLSEKAKPALEDLRQGLLPVLESF

SEQ ID NO: 268 - protein, 32 aa, Homo Sapiens
KATEHLSTLSEKAKPALEDLRQGLLPVLESFK

SEQ ID NO: 269 - protein, 32 aa, Homo Sapiens
ATEHLSTLSEKAKPALEDLRQGLLPVLESFKV

SEQ ID NO: 270 - protein, 32 aa, Homo Sapiens
TEHLSTLSEKAKPALEDLRQGLLPVLESFKVS

SEQ ID NO: 271 - protein, 32 aa, Homo Sapiens
EHLSTLSEKAKPALEDLRQGLLPVLESFKVSF

SEQ ID NO: 272 - protein, 32 aa, Homo Sapiens
HLSTLSEKAKPALEDLRQGLLPVLESFKVSFL

SEQ ID NO: 273 - protein, 32 aa, Homo Sapiens
LSTLSEKAKPALEDLRQGLLPVLESFKVSFLS

SEQ ID NO: 274 - protein, 32 aa, Homo Sapiens
STLSEKAKPALEDLRQGLLPVLESFKVSFLSA

SEQ ID NO: 275 - protein, 32 aa, Homo Sapiens
TLSEKAKPALEDLRQGLLPVLESFKVSFLSAL

SEQ ID NO: 276 - protein, 32 aa, Homo Sapiens
LSEKAKPALEDLRQGLLPVLESFKVSFLSALE

SEQ ID NO: 277 - protein, 32 aa, Homo Sapiens
SEKAKPALEDLRQGLLPVLESFKVSFLSALEE

SEQ ID NO: 278 - protein, 32 aa, Homo Sapiens
EKAKPALEDLRQGLLPVLESFKVSFLSALEEY

SEQ ID NO: 279 - protein, 32 aa, Homo Sapiens
KAKPALEDLRQGLLPVLESFKVSFLSALEEYT

SEQ ID NO: 280 - protein, 32 aa, Homo Sapiens
AKPALEDLRQGLLPVLESFKVSFLSALEEYTK

SEQ ID NO: 281 - protein, 32 aa, Homo Sapiens
KPALEDLRQGLLPVLESFKVSFLSALEEYTKK

SEQ ID NO: 282 - protein, 32 aa, Homo Sapiens
PALEDLRQGLLPVLESFKVSFLSALEEYTKKL

SEQ ID NO: 283 - protein, 32 aa, Homo Sapiens
ALEDLRQGLLPVLESFKVSFLSALEEYTKKLN

SEQ ID NO: 284 - protein, 32 aa, Homo Sapiens
LEDLRQGLLPVLESFKVSFLSALEEYTKKLNT

SEQ ID NO: 285 - protein, 32 aa, Homo Sapiens
EDLRQGLLPVLESFKVSFLSALEEYTKKLNTQ

SEQ ID NO: 286 - protein, 31 aa, Homo Sapiens
AEYHAKATEHLSTLSEKAKPALEDLRQGLLP

SEQ ID NO: 287 - protein, 31 aa, Homo Sapiens
EYHAKATEHLSTLSEKAKPALEDLRQGLLPV

SEQ ID NO: 288 - protein, 31 aa, Homo Sapiens
YHAKATEHLSTLSEKAKPALEDLRQGLLPVL

SEQ ID NO: 289 - protein, 31 aa, Homo Sapiens
HAKATEHLSTLSEKAKPALEDLRQGLLPVLE

SEQ ID NO: 290 - protein, 31 aa, Homo Sapiens
AKATEHLSTLSEKAKPALEDLRQGLLPVLES

SEQ ID NO: 291 - protein, 31 aa, Homo Sapiens
KATEHLSTLSEKAKPALEDLRQGLLPVLESF

SEQ ID NO: 292 - protein, 31 aa, Homo Sapiens
ATEHLSTLSEKAKPALEDLRQGLLPVLESFK

SEQ ID NO: 293 - protein, 31 aa, Homo Sapiens
TEHLSTLSEKAKPALEDLRQGLLPVLESFKV

SEQ ID NO: 294 - protein, 31 aa, Homo Sapiens
EHLSTLSEKAKPALEDLRQGLLPVLESFKVS

SEQ ID NO: 295 - protein, 31 aa, Homo Sapiens
HLSTLSEKAKPALEDLRQGLLPVLESFKVSF

SEQ ID NO: 296 - protein, 31 aa, Homo Sapiens
LSTLSEKAKPALEDLRQGLLPVLESFKVSFL

SEQ ID NO: 297 - protein, 31 aa, Homo Sapiens
STLSEKAKPALEDLRQGLLPVLESFKVSFLS

SEQ ID NO: 298 - protein, 31 aa, Homo Sapiens
TLSEKAKPALEDLRQGLLPVLESFKVSFLSA

SEQ ID NO: 299 - protein, 31 aa, Homo Sapiens
LSEKAKPALEDLRQGLLPVLESFKVSFLSAL

SEQ ID NO: 300 - protein, 31 aa, Homo Sapiens
SEKAKPALEDLRQGLLPVLESFKVSFLSALE

SEQ ID NO: 301 - protein, 31 aa, Homo Sapiens
EKAKPALEDLRQGLLPVLESFKVSFLSALEE

SEQ ID NO: 302 - protein, 31 aa, Homo Sapiens
KAKPALEDLRQGLLPVLESFKVSFLSALEEY

SEQ ID NO: 303 - protein, 31 aa, Homo Sapiens
AKPALEDLRQGLLPVLESFKVSFLSALEEYT

SEQ ID NO: 304 - protein, 31 aa, Homo Sapiens
KPALEDLRQGLLPVLESFKVSFLSALEEYTK

SEQ ID NO: 305 - protein, 31 aa, Homo Sapiens
PALEDLRQGLLPVLESFKVSFLSALEEYTKK

SEQ ID NO: 306 - protein, 31 aa, Homo Sapiens
ALEDLRQGLLPVLESFKVSFLSALEEYTKKL

SEQ ID NO: 307 - protein, 31 aa, Homo Sapiens
LEDLRQGLLPVLESFKVSFLSALEEYTKKLN

SEQ ID NO: 308 - protein, 31 aa, Homo Sapiens
EDLRQGLLPVLESFKVSFLSALEEYTKKLNT

SEQ ID NO: 309 - protein, 31 aa, Homo Sapiens
DLRQGLLPVLESFKVSFLSALEEYTKKLNTQ

SEQ ID NO: 310 - protein, 30 aa, Homo Sapiens
AEYHAKATEHLSTLSEKAKPALEDLRQGLL

SEQ ID NO: 311 - protein, 30 aa, Homo Sapiens
EYHAKATEHLSTLSEKAKPALEDLRQGLLP

SEQ ID NO: 312 - protein, 30 aa, Homo Sapiens
YHAKATEHLSTLSEKAKPALEDLRQGLLPV

SEQ ID NO: 313 - protein, 30 aa, Homo Sapiens
HAKATEHLSTLSEKAKPALEDLRQGLLPVL

SEQ ID NO: 314 - protein, 30 aa, Homo Sapiens
AKATEHLSTLSEKAKPALEDLRQGLLPVLE

SEQ ID NO: 315 - protein, 30 aa, Homo Sapiens
KATEHLSTLSEKAKPALEDLRQGLLPVLES

SEQ ID NO: 316 - protein, 30 aa, Homo Sapiens
ATEHLSTLSEKAKPALEDLRQGLLPVLESF

SEQ ID NO: 317 - protein, 30 aa, Homo Sapiens
TEHLSTLSEKAKPALEDLRQGLLPVLESFK

SEQ ID NO: 318 - protein, 30 aa, Homo Sapiens
EHLSTLSEKAKPALEDLRQGLLPVLESFKV

SEQ ID NO: 319 - protein, 30 aa, Homo Sapiens
HLSTLSEKAKPALEDLRQGLLPVLESFKVS

SEQ ID NO: 320 - protein, 30 aa, Homo Sapiens
LSTLSEKAKPALEDLRQGLLPVLESFKVSF

SEQ ID NO: 321 - protein, 30 aa, Homo Sapiens
STLSEKAKPALEDLRQGLLPVLESFKVSFL

SEQ ID NO: 322 - protein, 30 aa, Homo Sapiens
TLSEKAKPALEDLRQGLLPVLESFKVSFLS

SEQ ID NO: 323 - protein, 30 aa, Homo Sapiens
LSEKAKPALEDLRQGLLPVLESFKVSFLSA

SEQ ID NO: 324 - protein, 30 aa, Homo Sapiens
SEKAKPALEDLRQGLLPVLESFKVSFLSAL

SEQ ID NO: 325 - protein, 30 aa, Homo Sapiens
EKAKPALEDLRQGLLPVLESFKVSFLSALE

SEQ ID NO: 326 - protein, 30 aa, Homo Sapiens
KAKPALEDLRQGLLPVLESFKVSFLSALEE

SEQ ID NO: 327 - protein, 30 aa, Homo Sapiens
AKPALEDLRQGLLPVLESFKVSFLSALEEY

SEQ ID NO: 328 - protein, 30 aa, Homo Sapiens
KPALEDLRQGLLPVLESFKVSFLSALEEYT

SEQ ID NO: 329 - protein, 30 aa, Homo Sapiens
PALEDLRQGLLPVLESFKVSFLSALEEYTK

SEQ ID NO: 330 - protein, 30 aa, Homo Sapiens
ALEDLRQGLLPVLESFKVSFLSALEEYTKK

SEQ ID NO: 331 - protein, 30 aa, Homo Sapiens
LEDLRQGLLPVLESFKVSFLSALEEYTKKL

SEQ ID NO: 332 - protein, 30 aa, Homo Sapiens
EDLRQGLLPVLESFKVSFLSALEEYTKKLN

SEQ ID NO: 333 - protein, 30 aa, Homo Sapiens
DLRQGLLPVLESFKVSFLSALEEYTKKLNT

SEQ ID NO: 334 - protein, 30 aa, Homo Sapiens
LRQGLLPVLESFKVSFLSALEEYTKKLNTQ

SEQ ID NO: 335 - protein, 29 aa, Homo Sapiens
AEYHAKATEHLSTLSEKAKPALEDLRQGL

SEQ ID NO: 336 - protein, 29 aa, Homo Sapiens
EYHAKATEHLSTLSEKAKPALEDLRQGLL

SEQ ID NO: 337 - protein, 29 aa, Homo Sapiens
YHAKATEHLSTLSEKAKPALEDLRQGLLP

SEQ ID NO: 338 - protein, 29 aa, Homo Sapiens
HAKATEHLSTLSEKAKPALEDLRQGLLPV

SEQ ID NO: 339 - protein, 29 aa, Homo Sapiens
AKATEHLSTLSEKAKPALEDLRQGLLPVL

SEQ ID NO: 340 - protein, 29 aa, Homo Sapiens
KATEHLSTLSEKAKPALEDLRQGLLPVLE

SEQ ID NO: 341 - protein, 29 aa, Homo Sapiens
ATEHLSTLSEKAKPALEDLRQGLLPVLES

SEQ ID NO: 342 - protein, 29 aa, Homo Sapiens
TEHLSTLSEKAKPALEDLRQGLLPVLESF

SEQ ID NO: 343 - protein, 29 aa, Homo Sapiens
EHLSTLSEKAKPALEDLRQGLLPVLESFK

SEQ ID NO: 344 - protein, 29 aa, Homo Sapiens
HLSTLSEKAKPALEDLRQGLLPVLESFKV

SEQ ID NO: 345 - protein, 29 aa, Homo Sapiens
LSTLSEKAKPALEDLRQGLLPVLESFKVS

SEQ ID NO: 346 - protein, 29 aa, Homo Sapiens
STLSEKAKPALEDLRQGLLPVLESFKVSF

SEQ ID NO: 347 - protein, 29 aa, Homo Sapiens
TLSEKAKPALEDLRQGLLPVLESFKVSFL

SEQ ID NO: 348 - protein, 29 aa, Homo Sapiens
LSEKAKPALEDLRQGLLPVLESFKVSFLS

SEQ ID NO: 349 - protein, 29 aa, Homo Sapiens
SEKAKPALEDLRQGLLPVLESFKVSFLSA

SEQ ID NO: 350 - protein, 29 aa, Homo Sapiens
EKAKPALEDLRQGLLPVLESFKVSFLSAL

SEQ ID NO: 351 - protein, 29 aa, Homo Sapiens
KAKPALEDLRQGLLPVLESFKVSFLSALE

SEQ ID NO: 352 - protein, 29 aa, Homo Sapiens
AKPALEDLRQGLLPVLESFKVSFLSALEE

SEQ ID NO: 353 - protein, 29 aa, Homo Sapiens
KPALEDLRQGLLPVLESFKVSFLSALEEY

SEQ ID NO: 354 - protein, 29 aa, Homo Sapiens
PALEDLRQGLLPVLESFKVSFLSALEEYT

SEQ ID NO: 355 - protein, 29 aa, Homo Sapiens
ALEDLRQGLLPVLESFKVSFLSALEEYTK

SEQ ID NO: 356 - protein, 29 aa, Homo Sapiens
LEDLRQGLLPVLESFKVSFLSALEEYTKK

SEQ ID NO: 357 - protein, 29 aa, Homo Sapiens
EDLRQGLLPVLESFKVSFLSALEEYTKKL

SEQ ID NO: 358 - protein, 29 aa, Homo Sapiens
DLRQGLLPVLESFKVSFLSALEEYTKKLN

SEQ ID NO: 359 - protein, 29 aa, Homo Sapiens
LRQGLLPVLESFKVSFLSALEEYTKKLNT

SEQ ID NO: 360 - protein, 29 aa, Homo Sapiens
RQGLLPVLESFKVSFLSALEEYTKKLNTQ

SEQ ID NO: 361 - protein, 28 aa, Homo Sapiens
AEYHAKATEHLSTLSEKAKPALEDLRQG

SEQ ID NO: 362 - protein, 28 aa, Homo Sapiens
EYHAKATEHLSTLSEKAKPALEDLRQGL

SEQ ID NO: 363 - protein, 28 aa, Homo Sapiens
YHAKATEHLSTLSEKAKPALEDLRQGLL

SEQ ID NO: 364 - protein, 28 aa, Homo Sapiens
HAKATEHLSTLSEKAKPALEDLRQGLLP

SEQ ID NO: 365 - protein, 28 aa, Homo Sapiens
AKATEHLSTLSEKAKPALEDLRQGLLPV

SEQ ID NO: 366 - protein, 28 aa, Homo Sapiens
KATEHLSTLSEKAKPALEDLRQGLLPVL

SEQ ID NO: 367 - protein, 28 aa, Homo Sapiens
ATEHLSTLSEKAKPALEDLRQGLLPVLE

SEQ ID NO: 368 - protein, 28 aa, Homo Sapiens
TEHLSTLSEKAKPALEDLRQGLLPVLES

SEQ ID NO: 369 - protein, 28 aa, Homo Sapiens
EHLSTLSEKAKPALEDLRQGLLPVLESF

SEQ ID NO: 370 - protein, 28 aa, Homo Sapiens
HLSTLSEKAKPALEDLRQGLLPVLESFK

SEQ ID NO: 371 - protein, 28 aa, Homo Sapiens
LSTLSEKAKPALEDLRQGLLPVLESFKV

SEQ ID NO: 372 - protein, 28 aa, Homo Sapiens
STLSEKAKPALEDLRQGLLPVLESFKVS

SEQ ID NO: 373 - protein, 28 aa, Homo Sapiens
TLSEKAKPALEDLRQGLLPVLESFKVSF

SEQ ID NO: 374 - protein, 28 aa, Homo Sapiens
LSEKAKPALEDLRQGLLPVLESFKVSFL

SEQ ID NO: 375 - protein, 28 aa, Homo Sapiens
SEKAKPALEDLRQGLLPVLESFKVSFLS

SEQ ID NO: 376 - protein, 28 aa, Homo Sapiens
EKAKPALEDLRQGLLPVLESFKVSFLSA

SEQ ID NO: 377 - protein, 28 aa, Homo Sapiens
KAKPALEDLRQGLLPVLESFKVSFLSAL

SEQ ID NO: 378 - protein, 28 aa, Homo Sapiens
AKPALEDLRQGLLPVLESFKVSFLSALE

SEQ ID NO: 379 - protein, 28 aa, Homo Sapiens
KPALEDLRQGLLPVLESFKVSFLSALEE

SEQ ID NO: 380 - protein, 28 aa, Homo Sapiens
PALEDLRQGLLPVLESFKVSFLSALEEY

SEQ ID NO: 381 - protein, 28 aa, Homo Sapiens
ALEDLRQGLLPVLESFKVSFLSALEEYT

SEQ ID NO: 382 - protein, 28 aa, Homo Sapiens
LEDLRQGLLPVLESFKVSFLSALEEYTK

SEQ ID NO: 383 - protein, 28 aa, Homo Sapiens
EDLRQGLLPVLESFKVSFLSALEEYTKK

SEQ ID NO: 384 - protein, 28 aa, Homo Sapiens
DLRQGLLPVLESFKVSFLSALEEYTKKL

SEQ ID NO: 385 - protein, 28 aa, Homo Sapiens
LRQGLLPVLESFKVSFLSALEEYTKKLN

SEQ ID NO: 386 - protein, 28 aa, Homo Sapiens
RQGLLPVLESFKVSFLSALEEYTKKLNT

SEQ ID NO: 387 - protein, 28 aa, Homo Sapiens
QGLLPVLESFKVSFLSALEEYTKKLNTQ

SEQ ID NO: 388 - protein, 27 aa, Homo Sapiens
EYHAKATEHLSTLSEKAKPALEDLRQG

SEQ ID NO: 389 - protein, 27 aa, Homo Sapiens
YHAKATEHLSTLSEKAKPALEDLRQGL

SEQ ID NO: 390 - protein, 27 aa, Homo Sapiens
HAKATEHLSTLSEKAKPALEDLRQGLL

SEQ ID NO: 391 - protein, 27 aa, Homo Sapiens
AKATEHLSTLSEKAKPALEDLRQGLLP

SEQ ID NO: 392 - protein, 27 aa, Homo Sapiens
KATEHLSTLSEKAKPALEDLRQGLLPV

SEQ ID NO: 393 - protein, 27 aa, Homo Sapiens
ATEHLSTLSEKAKPALEDLRQGLLPVL

SEQ ID NO: 394 - protein, 27 aa, Homo Sapiens
TEHLSTLSEKAKPALEDLRQGLLPVLE

SEQ ID NO: 395 - protein, 27 aa, Homo Sapiens
EHLSTLSEKAKPALEDLRQGLLPVLES

SEQ ID NO: 396 - protein, 27 aa, Homo Sapiens
HLSTLSEKAKPALEDLRQGLLPVLESF

SEQ ID NO: 397 - protein, 27 aa, Homo Sapiens
LSTLSEKAKPALEDLRQGLLPVLESFK

SEQ ID NO: 398 - protein, 27 aa, Homo Sapiens
STLSEKAKPALEDLRQGLLPVLESFKV

SEQ ID NO: 399 - protein, 27 aa, Homo Sapiens
TLSEKAKPALEDLRQGLLPVLESFKVS

SEQ ID NO: 400 - protein, 27 aa, Homo Sapiens
LSEKAKPALEDLRQGLLPVLESFKVSF

SEQ ID NO: 401 - protein, 27 aa, Homo Sapiens
SEKAKPALEDLRQGLLPVLESFKVSFL

SEQ ID NO: 402 - protein, 27 aa, Homo Sapiens
EKAKPALEDLRQGLLPVLESFKVSFLS

SEQ ID NO: 403 - protein, 27 aa, Homo Sapiens
KAKPALEDLRQGLLPVLESFKVSFLSA

SEQ ID NO: 404 - protein, 27 aa, Homo Sapiens
AKPALEDLRQGLLPVLESFKVSFLSAL

SEQ ID NO: 405 - protein, 27 aa, Homo Sapiens
KPALEDLRQGLLPVLESFKVSFLSALE

SEQ ID NO: 406 - protein, 27 aa, Homo Sapiens
PALEDLRQGLLPVLESFKVSFLSALEE

SEQ ID NO: 407 - protein, 27 aa, Homo Sapiens
ALEDLRQGLLPVLESFKVSFLSALEEY

SEQ ID NO: 408 - protein, 27 aa, Homo Sapiens
LEDLRQGLLPVLESFKVSFLSALEEYT

SEQ ID NO: 409 - protein, 27 aa, Homo Sapiens
EDLRQGLLPVLESFKVSFLSALEEYTK

SEQ ID NO: 410 - protein, 27 aa, Homo Sapiens
DLRQGLLPVLESFKVSFLSALEEYTKK

SEQ ID NO: 411 - protein, 27 aa, Homo Sapiens
LRQGLLPVLESFKVSFLSALEEYTKKL

SEQ ID NO: 412 - protein, 27 aa, Homo Sapiens
RQGLLPVLESFKVSFLSALEEYTKKLN

SEQ ID NO: 413 - protein, 27 aa, Homo Sapiens
QGLLPVLESFKVSFLSALEEYTKKLNT

SEQ ID NO: 414 - protein, 26 aa, Homo Sapiens
AEYHAKATEHLSTLSEKAKPALEDLR

SEQ ID NO: 415 - protein, 26 aa, Homo Sapiens
EYHAKATEHLSTLSEKAKPALEDLRQ

SEQ ID NO: 416 - protein, 26 aa, Homo Sapiens
YHAKATEHLSTLSEKAKPALEDLRQG

SEQ ID NO: 417 - protein, 26 aa, Homo Sapiens
HAKATEHLSTLSEKAKPALEDLRQGL

SEQ ID NO: 418 - protein, 26 aa, Homo Sapiens
AKATEHLSTLSEKAKPALEDLRQGLL

SEQ ID NO: 419 - protein, 26 aa, Homo Sapiens
KATEHLSTLSEKAKPALEDLRQGLLP

SEQ ID NO: 420 - protein, 26 aa, Homo Sapiens
ATEHLSTLSEKAKPALEDLRQGLLPV

SEQ ID NO: 421 - protein, 26 aa, Homo Sapiens
TEHLSTLSEKAKPALEDLRQGLLPVL

SEQ ID NO: 422 - protein, 26 aa, Homo Sapiens
EHLSTLSEKAKPALEDLRQGLLPVLE

SEQ ID NO: 423 - protein, 26 aa, Homo Sapiens
HLSTLSEKAKPALEDLRQGLLPVLES

SEQ ID NO: 424 - protein, 26 aa, Homo Sapiens
LSTLSEKAKPALEDLRQGLLPVLESF

SEQ ID NO: 425 - protein, 26 aa, Homo Sapiens
STLSEKAKPALEDLRQGLLPVLESFK

SEQ ID NO: 426 - protein, 26 aa, Homo Sapiens
TLSEKAKPALEDLRQGLLPVLESFKV

SEQ ID NO: 427 - protein, 26 aa, Homo Sapiens
LSEKAKPALEDLRQGLLPVLESFKVS

SEQ ID NO: 428 - protein, 26 aa, Homo Sapiens
SEKAKPALEDLRQGLLPVLESFKVSF

SEQ ID NO: 429 - protein, 26 aa, Homo Sapiens
EKAKPALEDLRQGLLPVLESFKVSFL

SEQ ID NO: 430 - protein, 26 aa, Homo Sapiens
KAKPALEDLRQGLLPVLESFKVSFLS

SEQ ID NO: 431 - protein, 26 aa, Homo Sapiens
AKPALEDLRQGLLPVLESFKVSFLSA

SEQ ID NO: 432 - protein, 26 aa, Homo Sapiens
KPALEDLRQGLLPVLESFKVSFLSAL

SEQ ID NO: 433 - protein, 26 aa, Homo Sapiens
PALEDLRQGLLPVLESFKVSFLSALE

SEQ ID NO: 434 - protein, 26 aa, Homo Sapiens
ALEDLRQGLLPVLESFKVSFLSALEE

SEQ ID NO: 435 - protein, 26 aa, Homo Sapiens
LEDLRQGLLPVLESFKVSFLSALEEY

SEQ ID NO: 436 - protein, 26 aa, Homo Sapiens
EDLRQGLLPVLESFKVSFLSALEEYT

SEQ ID NO: 437 - protein, 26 aa, Homo Sapiens
DLRQGLLPVLESFKVSFLSALEEYTK

SEQ ID NO: 438 - protein, 26 aa, Homo Sapiens
LRQGLLPVLESFKVSFLSALEEYTKK

SEQ ID NO: 439 - protein, 26 aa, Homo Sapiens
RQGLLPVLESFKVSFLSALEEYTKKL

SEQ ID NO: 440 - protein, 26 aa, Homo Sapiens
QGLLPVLESFKVSFLSALEEYTKKLN

SEQ ID NO: 441 - protein, 26 aa, Homo Sapiens
GLLPVLESFKVSFLSALEEYTKKLNT

SEQ ID NO: 442 - protein, 26 aa, Homo Sapiens
LLPVLESFKVSFLSALEEYTKKLNTQ

SEQ ID NO: 443 - protein, 25 aa, Homo Sapiens
AEYHAKATEHLSTLSEKAKPALEDL

SEQ ID NO: 444 - protein, 25 aa, Homo Sapiens
EYHAKATEHLSTLSEKAKPALEDLR

SEQ ID NO: 445 - protein, 25 aa, Homo Sapiens
YHAKATEHLSTLSEKAKPALEDLRQ

SEQ ID NO: 446 - protein, 25 aa, Homo Sapiens
HAKATEHLSTLSEKAKPALEDLRQG

SEQ ID NO: 447 - protein, 25 aa, Homo Sapiens
AKATEHLSTLSEKAKPALEDLRQGL

SEQ ID NO: 448 - protein, 25 aa, Homo Sapiens
KATEHLSTLSEKAKPALEDLRQGLL

SEQ ID NO: 449 - protein, 25 aa, Homo Sapiens
ATEHLSTLSEKAKPALEDLRQGLLP

SEQ ID NO: 450 - protein, 25 aa, Homo Sapiens
TEHLSTLSEKAKPALEDLRQGLLPV

SEQ ID NO: 451 - protein, 25 aa, Homo Sapiens
EHLSTLSEKAKPALEDLRQGLLPVL

SEQ ID NO: 452 - protein, 25 aa, Homo Sapiens
HLSTLSEKAKPALEDLRQGLLPVLE

SEQ ID NO: 453 - protein, 25 aa, Homo Sapiens
LSTLSEKAKPALEDLRQGLLPVLES

SEQ ID NO: 454 - protein, 25 aa, Homo Sapiens
STLSEKAKPALEDLRQGLLPVLESF

SEQ ID NO: 455 - protein, 25 aa, Homo Sapiens
TLSEKAKPALEDLRQGLLPVLESFK

SEQ ID NO: 456 - protein, 25 aa, Homo Sapiens
LSEKAKPALEDLRQGLLPVLESFKV

SEQ ID NO: 457 - protein, 25 aa, Homo Sapiens
SEKAKPALEDLRQGLLPVLESFKVS

SEQ ID NO: 458 - protein, 25 aa, Homo Sapiens
EKAKPALEDLRQGLLPVLESFKVSF

SEQ ID NO: 459 - protein, 25 aa, Homo Sapiens
KAKPALEDLRQGLLPVLESFKVSFL

SEQ ID NO: 460 - protein, 25 aa, Homo Sapiens
AKPALEDLRQGLLPVLESFKVSFLS

SEQ ID NO: 461 - protein, 25 aa, Homo Sapiens
KPALEDLRQGLLPVLESFKVSFLSA

SEQ ID NO: 462 - protein, 25 aa, Homo Sapiens
PALEDLRQGLLPVLESFKVSFLSAL

SEQ ID NO: 463 - protein, 25 aa, Homo Sapiens
ALEDLRQGLLPVLESFKVSFLSALE

SEQ ID NO: 464 - protein, 25 aa, Homo Sapiens
LEDLRQGLLPVLESFKVSFLSALEE

SEQ ID NO: 465 - protein, 25 aa, Homo Sapiens
EDLRQGLLPVLESFKVSFLSALEEY

SEQ ID NO: 466 - protein, 25 aa, Homo Sapiens
DLRQGLLPVLESFKVSFLSALEEYT

SEQ ID NO: 467 - protein, 25 aa, Homo Sapiens
LRQGLLPVLESFKVSFLSALEEYTK

SEQ ID NO: 468 - protein, 25 aa, Homo Sapiens
RQGLLPVLESFKVSFLSALEEYTKK

SEQ ID NO: 469 - protein, 25 aa, Homo Sapiens
QGLLPVLESFKVSFLSALEEYTKKL

SEQ ID NO: 470 - protein, 25 aa, Homo Sapiens
GLLPVLESFKVSFLSALEEYTKKLN

SEQ ID NO: 471 - protein, 25 aa, Homo Sapiens
LLPVLESFKVSFLSALEEYTKKLNT

SEQ ID NO: 472 - protein, 25 aa, Homo Sapiens
LPVLESFKVSFLSALEEYTKKLNTQ

SEQ ID NO: 473 - protein, 24 aa, Homo Sapiens
AEYHAKATEHLSTLSEKAKPALED

SEQ ID NO: 474 - protein, 24 aa, Homo Sapiens
EYHAKATEHLSTLSEKAKPALEDL

SEQ ID NO: 475 - protein, 24 aa, Homo Sapiens
YHAKATEHLSTLSEKAKPALEDLR

SEQ ID NO: 476 - protein, 24 aa, Homo Sapiens
HAKATEHLSTLSEKAKPALEDLRQ

SEQ ID NO: 477 - protein, 24 aa, Homo Sapiens
AKATEHLSTLSEKAKPALEDLRQG

SEQ ID NO: 478 - protein, 24 aa, Homo Sapiens
KATEHLSTLSEKAKPALEDLRQGL

SEQ ID NO: 479 - protein, 24 aa, Homo Sapiens
ATEHLSTLSEKAKPALEDLRQGLL

SEQ ID NO: 480 - protein, 24 aa, Homo Sapiens
TEHLSTLSEKAKPALEDLRQGLLP

SEQ ID NO: 481 - protein, 24 aa, Homo Sapiens
EHLSTLSEKAKPALEDLRQGLLPV

SEQ ID NO: 482 - protein, 24 aa, Homo Sapiens
HLSTLSEKAKPALEDLRQGLLPVL

SEQ ID NO: 483 - protein, 24 aa, Homo Sapiens
LSTLSEKAKPALEDLRQGLLPVLE

SEQ ID NO: 484 - protein, 24 aa, Homo Sapiens
STLSEKAKPALEDLRQGLLPVLES

SEQ ID NO: 485 - protein, 24 aa, Homo Sapiens
TLSEKAKPALEDLRQGLLPVLESF

SEQ ID NO: 486 - protein, 24 aa, Homo Sapiens
LSEKAKPALEDLRQGLLPVLESFK

SEQ ID NO: 487 - protein, 24 aa, Homo Sapiens
SEKAKPALEDLRQGLLPVLESFKV

SEQ ID NO: 488 - protein, 24 aa, Homo Sapiens
EKAKPALEDLRQGLLPVLESFKVS

SEQ ID NO: 489 - protein, 24 aa, Homo Sapiens
KAKPALEDLRQGLLPVLESFKVSF

SEQ ID NO: 490 - protein, 24 aa, Homo Sapiens
AKPALEDLRQGLLPVLESFKVSFL

SEQ ID NO: 491 - protein, 24 aa, Homo Sapiens
KPALEDLRQGLLPVLESFKVSFLS

SEQ ID NO: 492 - protein, 24 aa, Homo Sapiens
PALEDLRQGLLPVLESFKVSFLSA

SEQ ID NO: 493 - protein, 24 aa, Homo Sapiens
ALEDLRQGLLPVLESFKVSFLSAL

SEQ ID NO: 494 - protein, 24 aa, Homo Sapiens
LEDLRQGLLPVLESFKVSFLSALE

SEQ ID NO: 495 - protein, 24 aa, Homo Sapiens
EDLRQGLLPVLESFKVSFLSALEE

SEQ ID NO: 496 - protein, 24 aa, Homo Sapiens
DLRQGLLPVLESFKVSFLSALEEY

SEQ ID NO: 497 - protein, 24 aa, Homo Sapiens
LRQGLLPVLESFKVSFLSALEEYT

SEQ ID NO: 498 - protein, 24 aa, Homo Sapiens
RQGLLPVLESFKVSFLSALEEYTK

SEQ ID NO: 499 - protein, 24 aa, Homo Sapiens
QGLLPVLESFKVSFLSALEEYTKK

SEQ ID NO: 500 - protein, 24 aa, Homo Sapiens
GLLPVLESFKVSFLSALEEYTKKL

SEQ ID NO: 501 - protein, 24 aa, Homo Sapiens
LLPVLESFKVSFLSALEEYTKKLN

SEQ ID NO: 502 - protein, 24 aa, Homo Sapiens
LPVLESFKVSFLSALEEYTKKLNT

SEQ ID NO: 503 - protein, 24 aa, Homo Sapiens
PVLESFKVSFLSALEEYTKKLNTQ

SEQ ID NO: 504 - protein, 23 aa, Homo Sapiens
AEYHAKATEHLSTLSEKAKPALE

SEQ ID NO: 505 - protein, 23 aa, Homo Sapiens
EYHAKATEHLSTLSEKAKPALED

SEQ ID NO: 506 - protein, 23 aa, Homo Sapiens
YHAKATEHLSTLSEKAKPALEDL

SEQ ID NO: 507 - protein, 23 aa, Homo Sapiens
HAKATEHLSTLSEKAKPALEDLR

SEQ ID NO: 508 - protein, 23 aa, Homo Sapiens
AKATEHLSTLSEKAKPALEDLRQ

SEQ ID NO: 509 - protein, 23 aa, Homo Sapiens
KATEHLSTLSEKAKPALEDLRQG

SEQ ID NO: 510 - protein, 23 aa, Homo Sapiens
ATEHLSTLSEKAKPALEDLRQGL

SEQ ID NO: 511 - protein, 23 aa, Homo Sapiens
TEHLSTLSEKAKPALEDLRQGLL

SEQ ID NO: 512 - protein, 23 aa, Homo Sapiens
EHLSTLSEKAKPALEDLRQGLLP

SEQ ID NO: 513 - protein, 23 aa, Homo Sapiens
HLSTLSEKAKPALEDLRQGLLPV

SEQ ID NO: 514 - protein, 23 aa, Homo Sapiens
LSTLSEKAKPALEDLRQGLLPVL

SEQ ID NO: 515 - protein, 23 aa, Homo Sapiens
STLSEKAKPALEDLRQGLLPVLE

SEQ ID NO: 516 - protein, 23 aa, Homo Sapiens
TLSEKAKPALEDLRQGLLPVLES

SEQ ID NO: 517 - protein, 23 aa, Homo Sapiens
LSEKAKPALEDLRQGLLPVLESF

SEQ ID NO: 518 - protein, 23 aa, Homo Sapiens
SEKAKPALEDLRQGLLPVLESFK

SEQ ID NO: 519 - protein, 23 aa, Homo Sapiens
EKAKPALEDLRQGLLPVLESFKV

SEQ ID NO: 520 - protein, 23 aa, Homo Sapiens
KAKPALEDLRQGLLPVLESFKVS

SEQ ID NO: 521 - protein, 23 aa, Homo Sapiens
AKPALEDLRQGLLPVLESFKVSF

SEQ ID NO: 522 - protein, 23 aa, Homo Sapiens
KPALEDLRQGLLPVLESFKVSFL

SEQ ID NO: 523 - protein, 23 aa, Homo Sapiens
PALEDLRQGLLPVLESFKVSFLS

SEQ ID NO: 524 - protein, 23 aa, Homo Sapiens
ALEDLRQGLLPVLESFKVSFLSA

SEQ ID NO: 525 - protein, 23 aa, Homo Sapiens
LEDLRQGLLPVLESFKVSFLSAL

SEQ ID NO: 526 - protein, 23 aa, Homo Sapiens
EDLRQGLLPVLESFKVSFLSALE

SEQ ID NO: 527 - protein, 23 aa, Homo Sapiens
DLRQGLLPVLESFKVSFLSALEE

SEQ ID NO: 528 - protein, 23 aa, Homo Sapiens
LRQGLLPVLESFKVSFLSALEEY

SEQ ID NO: 529 - protein, 23 aa, Homo Sapiens
RQGLLPVLESFKVSFLSALEEYT

SEQ ID NO: 530 - protein, 23 aa, Homo Sapiens
QGLLPVLESFKVSFLSALEEYTK

SEQ ID NO: 531 - protein, 23 aa, Homo Sapiens
GLLPVLESFKVSFLSALEEYTKK

SEQ ID NO: 532 - protein, 23 aa, Homo Sapiens
LLPVLESFKVSFLSALEEYTKKL

SEQ ID NO: 533 - protein, 23 aa, Homo Sapiens
LPVLESFKVSFLSALEEYTKKLN

SEQ ID NO: 534 - protein, 23 aa, Homo Sapiens
PVLESFKVSFLSALEEYTKKLNT

SEQ ID NO: 535 - protein, 23 aa, Homo Sapiens
VLESFKVSFLSALEEYTKKLNTQ

SEQ ID NO: 536 - protein, 22 aa, Homo Sapiens
AEYHAKATEHLSTLSEKAKPAL

SEQ ID NO: 537 - protein, 22 aa, Homo Sapiens
EYHAKATEHLSTLSEKAKPALE

SEQ ID NO: 538 - protein, 22 aa, Homo Sapiens
YHAKATEHLSTLSEKAKPALED

SEQ ID NO: 539 - protein, 22 aa, Homo Sapiens
HAKATEHLSTLSEKAKPALEDL

SEQ ID NO: 540 - protein, 22 aa, Homo Sapiens
AKATEHLSTLSEKAKPALEDLR

SEQ ID NO: 541 - protein, 22 aa, Homo Sapiens
KATEHLSTLSEKAKPALEDLRQ

SEQ ID NO: 542 - protein, 22 aa, Homo Sapiens
ATEHLSTLSEKAKPALEDLRQG

SEQ ID NO: 543 - protein, 22 aa, Homo Sapiens
TEHLSTLSEKAKPALEDLRQGL

SEQ ID NO: 544 - protein, 22 aa, Homo Sapiens
EHLSTLSEKAKPALEDLRQGLL

SEQ ID NO: 545 - protein, 22 aa, Homo Sapiens
HLSTLSEKAKPALEDLRQGLLP

SEQ ID NO: 546 - protein, 22 aa, Homo Sapiens
LSTLSEKAKPALEDLRQGLLPV

SEQ ID NO: 547 - protein, 22 aa, Homo Sapiens
STLSEKAKPALEDLRQGLLPVL

SEQ ID NO: 548 - protein, 22 aa, Homo Sapiens
TLSEKAKPALEDLRQGLLPVLE

SEQ ID NO: 549 - protein, 22 aa, Homo Sapiens
LSEKAKPALEDLRQGLLPVLES

SEQ ID NO: 550 - protein, 22 aa, Homo Sapiens
SEKAKPALEDLRQGLLPVLESF

SEQ ID NO: 551 - protein, 22 aa, Homo Sapiens
EKAKPALEDLRQGLLPVLESFK

SEQ ID NO: 552 - protein, 22 aa, Homo Sapiens
KAKPALEDLRQGLLPVLESFKV

SEQ ID NO: 553 - protein, 22 aa, Homo Sapiens
AKPALEDLRQGLLPVLESFKVS

SEQ ID NO: 554 - protein, 22 aa, Homo Sapiens
KPALEDLRQGLLPVLESFKVSF

SEQ ID NO: 555 - protein, 22 aa, Homo Sapiens
PALEDLRQGLLPVLESFKVSFL

SEQ ID NO: 556 - protein, 22 aa, Homo Sapiens
ALEDLRQGLLPVLESFKVSFLS

SEQ ID NO: 557 - protein, 22 aa, Homo Sapiens
LEDLRQGLLPVLESFKVSFLSA

SEQ ID NO: 558 - protein, 22 aa, Homo Sapiens
EDLRQGLLPVLESFKVSFLSAL

SEQ ID NO: 559 - protein, 22 aa, Homo Sapiens
DLRQGLLPVLESFKVSFLSALE

SEQ ID NO: 560 - protein, 22 aa, Homo Sapiens
LRQGLLPVLESFKVSFLSALEE

SEQ ID NO: 561 - protein, 22 aa, Homo Sapiens
RQGLLPVLESFKVSFLSALEEY

SEQ ID NO: 562 - protein, 22 aa, Homo Sapiens
QGLLPVLESFKVSFLSALEEYT

SEQ ID NO: 563 - protein, 22 aa, Homo Sapiens
GLLPVLESFKVSFLSALEEYTK

SEQ ID NO: 564 - protein, 22 aa, Homo Sapiens
LLPVLESFKVSFLSALEEYTKK

SEQ ID NO: 565 - protein, 22 aa, Homo Sapiens
LPVLESFKVSFLSALEEYTKKL

SEQ ID NO: 566 - protein, 22 aa, Homo Sapiens
PVLESFKVSFLSALEEYTKKLN

SEQ ID NO: 567 - protein, 22 aa, Homo Sapiens
VLESFKVSFLSALEEYTKKLNT

SEQ ID NO: 568 - protein, 22 aa, Homo Sapiens
LESFKVSFLSALEEYTKKLNTQ

SEQ ID NO: 569 - protein, 21 aa, Homo Sapiens
AEYHAKATEHLSTLSEKAKPA

SEQ ID NO: 570 - protein, 21 aa, Homo Sapiens
EYHAKATEHLSTLSEKAKPAL

SEQ ID NO: 571 - protein, 21 aa, Homo Sapiens
YHAKATEHLSTLSEKAKPALE

SEQ ID NO: 572 - protein, 21 aa, Homo Sapiens
HAKATEHLSTLSEKAKPALED

SEQ ID NO: 573 - protein, 21 aa, Homo Sapiens
AKATEHLSTLSEKAKPALEDL

SEQ ID NO: 574 - protein, 21 aa, Homo Sapiens
KATEHLSTLSEKAKPALEDLR

SEQ ID NO: 575 - protein, 21 aa, Homo Sapiens
ATEHLSTLSEKAKPALEDLRQ

SEQ ID NO: 576 - protein, 21 aa, Homo Sapiens
TEHLSTLSEKAKPALEDLRQG

SEQ ID NO: 577 - protein, 21 aa, Homo Sapiens
EHLSTLSEKAKPALEDLRQGL

SEQ ID NO: 578 - protein, 21 aa, Homo Sapiens
HLSTLSEKAKPALEDLRQGLL

SEQ ID NO: 579 - protein, 21 aa, Homo Sapiens
LSTLSEKAKPALEDLRQGLLP

SEQ ID NO: 580 - protein, 21 aa, Homo Sapiens
STLSEKAKPALEDLRQGLLPV

SEQ ID NO: 581 - protein, 21 aa, Homo Sapiens
TLSEKAKPALEDLRQGLLPVL

SEQ ID NO: 582 - protein, 21 aa, Homo Sapiens
LSEKAKPALEDLRQGLLPVLE

SEQ ID NO: 583 - protein, 21 aa, Homo Sapiens
SEKAKPALEDLRQGLLPVLES

SEQ ID NO: 584 - protein, 21 aa, Homo Sapiens
EKAKPALEDLRQGLLPVLESF

SEQ ID NO: 585 - protein, 21 aa, Homo Sapiens
KAKPALEDLRQGLLPVLESFK

SEQ ID NO: 586 - protein, 21 aa, Homo Sapiens
AKPALEDLRQGLLPVLESFKV

SEQ ID NO: 587 - protein, 21 aa, Homo Sapiens
KPALEDLRQGLLPVLESFKVS

SEQ ID NO: 588 - protein, 21 aa, Homo Sapiens
PALEDLRQGLLPVLESFKVSF

SEQ ID NO: 589 - protein, 21 aa, Homo Sapiens
ALEDLRQGLLPVLESFKVSFL

SEQ ID NO: 590 - protein, 21 aa, Homo Sapiens
LEDLRQGLLPVLESFKVSFLS

SEQ ID NO: 591 - protein, 21 aa, Homo Sapiens
EDLRQGLLPVLESFKVSFLSA

SEQ ID NO: 592 - protein, 21 aa, Homo Sapiens
DLRQGLLPVLESFKVSFLSAL

SEQ ID NO: 593 - protein, 21 aa, Homo Sapiens
LRQGLLPVLESFKVSFLSALE

SEQ ID NO: 594 - protein, 21 aa, Homo Sapiens
RQGLLPVLESFKVSFLSALEE

SEQ ID NO: 595 - protein, 21 aa, Homo Sapiens
QGLLPVLESFKVSFLSALEEY

SEQ ID NO: 596 - protein, 21 aa, Homo Sapiens
GLLPVLESFKVSFLSALEEYT

SEQ ID NO: 597 - protein, 21 aa, Homo Sapiens
LLPVLESFKVSFLSALEEYTK

SEQ ID NO: 598 - protein, 21 aa, Homo Sapiens
LPVLESFKVSFLSALEEYTKK

SEQ ID NO: 599 - protein, 21 aa, Homo Sapiens
PVLESFKVSFLSALEEYTKKL

SEQ ID NO: 600 - protein, 21 aa, Homo Sapiens
VLESFKVSFLSALEEYTKKLN

SEQ ID NO: 601 - protein, 21 aa, Homo Sapiens
LESFKVSFLSALEEYTKKLNT

SEQ ID NO: 602 - protein, 21 aa, Homo Sapiens
ESFKVSFLSALEEYTKKLNTQ

SEQ ID NO: 603 - protein, 20 aa, Homo Sapiens
AEYHAKATEHLSTLSEKAKP

SEQ ID NO: 604 - protein, 20 aa, Homo Sapiens
EYHAKATEHLSTLSEKAKPA

SEQ ID NO: 605 - protein, 20 aa, Homo Sapiens
YHAKATEHLSTLSEKAKPAL

SEQ ID NO: 606 - protein, 20 aa, Homo Sapiens
HAKATEHLSTLSEKAKPALE

SEQ ID NO: 607 - protein, 20 aa, Homo Sapiens
AKATEHLSTLSEKAKPALED

SEQ ID NO: 608 - protein, 20 aa, Homo Sapiens
KATEHLSTLSEKAKPALEDL

SEQ ID NO: 609 - protein, 20 aa, Homo Sapiens
ATEHLSTLSEKAKPALEDLR

SEQ ID NO: 610 - protein, 20 aa, Homo Sapiens
TEHLSTLSEKAKPALEDLRQ

SEQ ID NO: 611 - protein, 20 aa, Homo Sapiens
EHLSTLSEKAKPALEDLRQG

SEQ ID NO: 612 - protein, 20 aa, Homo Sapiens
HLSTLSEKAKPALEDLRQGL

SEQ ID NO: 613 - protein, 20 aa, Homo Sapiens
LSTLSEKAKPALEDLRQGLL

SEQ ID NO: 614 - protein, 20 aa, Homo Sapiens
STLSEKAKPALEDLRQGLLP

SEQ ID NO: 615 - protein, 20 aa, Homo Sapiens
TLSEKAKPALEDLRQGLLPV

SEQ ID NO: 616 - protein, 20 aa, Homo Sapiens
LSEKAKPALEDLRQGLLPVL

SEQ ID NO: 617 - protein, 20 aa, Homo Sapiens
SEKAKPALEDLRQGLLPVLE

SEQ ID NO: 618 - protein, 20 aa, Homo Sapiens
EKAKPALEDLRQGLLPVLES

SEQ ID NO: 619 - protein, 20 aa, Homo Sapiens
KAKPALEDLRQGLLPVLESF

SEQ ID NO: 620 - protein, 20 aa, Homo Sapiens
AKPALEDLRQGLLPVLESFK

SEQ ID NO: 621 - protein, 20 aa, Homo Sapiens
KPALEDLRQGLLPVLESFKV

SEQ ID NO: 622 - protein, 20 aa, Homo Sapiens
PALEDLRQGLLPVLESFKVS

SEQ ID NO: 623 - protein, 20 aa, Homo Sapiens
ALEDLRQGLLPVLESFKVSF

SEQ ID NO: 624 - protein, 20 aa, Homo Sapiens
LEDLRQGLLPVLESFKVSFL

SEQ ID NO: 625 - protein, 20 aa, Homo Sapiens
EDLRQGLLPVLESFKVSFLS

SEQ ID NO: 626 - protein, 20 aa, Homo Sapiens
DLRQGLLPVLESFKVSFLSA

SEQ ID NO: 627 - protein, 20 aa, Homo Sapiens
LRQGLLPVLESFKVSFLSAL

SEQ ID NO: 628 - protein, 20 aa, Homo Sapiens
RQGLLPVLESFKVSFLSALE

SEQ ID NO: 629 - protein, 20 aa, Homo Sapiens
QGLLPVLESFKVSFLSALEE

SEQ ID NO: 630 - protein, 20 aa, Homo Sapiens
GLLPVLESFKVSFLSALEEY

SEQ ID NO: 631 - protein, 20 aa, Homo Sapiens
LLPVLESFKVSFLSALEEYT

SEQ ID NO: 632 - protein, 20 aa, Homo Sapiens
LPVLESFKVSFLSALEEYTK

SEQ ID NO: 633 - protein, 20 aa, Homo Sapiens
PVLESFKVSFLSALEEYTKK

SEQ ID NO: 634 - protein, 20 aa, Homo Sapiens
VLESFKVSFLSALEEYTKKL

SEQ ID NO: 635 - protein, 20 aa, Homo Sapiens
LESFKVSFLSALEEYTKKLN

SEQ ID NO: 636 - protein, 20 aa, Homo Sapiens
ESFKVSFLSALEEYTKKLNT

SEQ ID NO: 637 - protein, 20 aa, Homo Sapiens
SFKVSFLSALEEYTKKLNTQ

SEQ ID NO: 638 - protein, 19 aa, Homo Sapiens
AEYHAKATEHLSTLSEKAK

SEQ ID NO: 639 - protein, 19 aa, Homo Sapiens
EYHAKATEHLSTLSEKAKP

SEQ ID NO: 640 - protein, 19 aa, Homo Sapiens
YHAKATEHLSTLSEKAKPA

SEQ ID NO: 641 - protein, 19 aa, Homo Sapiens
HAKATEHLSTLSEKAKPAL

SEQ ID NO: 642 - protein, 19 aa, Homo Sapiens
AKATEHLSTLSEKAKPALE

SEQ ID NO: 643 - protein, 19 aa, Homo Sapiens
KATEHLSTLSEKAKPALED

SEQ ID NO: 644 - protein, 19 aa, Homo Sapiens
ATEHLSTLSEKAKPALEDL

SEQ ID NO: 645 - protein, 19 aa, Homo Sapiens
TEHLSTLSEKAKPALEDLR

SEQ ID NO: 646 - protein, 19 aa, Homo Sapiens
EHLSTLSEKAKPALEDLRQ

SEQ ID NO: 647 - protein, 19 aa, Homo Sapiens
HLSTLSEKAKPALEDLRQG

SEQ ID NO: 648 - protein, 19 aa, Homo Sapiens
LSTLSEKAKPALEDLRQGL

SEQ ID NO: 649 - protein, 19 aa, Homo Sapiens
STLSEKAKPALEDLRQGLL

SEQ ID NO: 650 - protein, 19 aa, Homo Sapiens
TLSEKAKPALEDLRQGLLP

SEQ ID NO: 651 - protein, 19 aa, Homo Sapiens
LSEKAKPALEDLRQGLLPV

SEQ ID NO: 652 - protein, 19 aa, Homo Sapiens
SEKAKPALEDLRQGLLPVL

SEQ ID NO: 653 - protein, 19 aa, Homo Sapiens
EKAKPALEDLRQGLLPVLE

SEQ ID NO: 654 - protein, 19 aa, Homo Sapiens
KAKPALEDLRQGLLPVLES

SEQ ID NO: 655 - protein, 19 aa, Homo Sapiens
AKPALEDLRQGLLPVLESF

SEQ ID NO: 656 - protein, 19 aa, Homo Sapiens
KPALEDLRQGLLPVLESFK

SEQ ID NO: 657 - protein, 19 aa, Homo Sapiens
PALEDLRQGLLPVLESFKV

SEQ ID NO: 658 - protein, 19 aa, Homo Sapiens
ALEDLRQGLLPVLESFKVS

SEQ ID NO: 659 - protein, 19 aa, Homo Sapiens
LEDLRQGLLPVLESFKVSF

SEQ ID NO: 660 - protein, 19 aa, Homo Sapiens
EDLRQGLLPVLESFKVSFL

SEQ ID NO: 661 - protein, 19 aa, Homo Sapiens
DLRQGLLPVLESFKVSFLS

SEQ ID NO: 662 - protein, 19 aa, Homo Sapiens
LRQGLLPVLESFKVSFLSA

SEQ ID NO: 663 - protein, 19 aa, Homo Sapiens
RQGLLPVLESFKVSFLSAL

SEQ ID NO: 664 - protein, 19 aa, Homo Sapiens
QGLLPVLESFKVSFLSALE

SEQ ID NO: 665 - protein, 19 aa, Homo Sapiens
GLLPVLESFKVSFLSALEE

SEQ ID NO: 666 - protein, 19 aa, Homo Sapiens
LLPVLESFKVSFLSALEEY

SEQ ID NO: 667 - protein, 19 aa, Homo Sapiens
LPVLESFKVSFLSALEEYT

SEQ ID NO: 668 - protein, 19 aa, Homo Sapiens
PVLESFKVSFLSALEEYTK

SEQ ID NO: 669 - protein, 19 aa, Homo Sapiens
VLESFKVSFLSALEEYTKK

SEQ ID NO: 670 - protein, 19 aa, Homo Sapiens
LESFKVSFLSALEEYTKKL

SEQ ID NO: 671 - protein, 19 aa, Homo Sapiens
ESFKVSFLSALEEYTKKLN

SEQ ID NO: 672 - protein, 19 aa, Homo Sapiens
SFKVSFLSALEEYTKKLNT

SEQ ID NO: 673 - protein, 19 aa, Homo Sapiens
FKVSFLSALEEYTKKLNTQ

SEQ ID NO: 674 - protein, 10 aa, Homo Sapiens
LEEYTKKLNT

SEQ ID NO: 675 - protein, 18 aa, Homo Sapiens
EYHAKATEHLSTLSEKAK

SEQ ID NO: 676 - protein, 18 aa, Homo Sapiens
YHAKATEHLSTLSEKAKP

SEQ ID NO: 677 - protein, 18 aa, Homo Sapiens
HAKATEHLSTLSEKAKPA

SEQ ID NO: 678 - protein, 18 aa, Homo Sapiens
AKATEHLSTLSEKAKPAL

SEQ ID NO: 679 - protein, 18 aa, Homo Sapiens
KATEHLSTLSEKAKPALE

SEQ ID NO: 680 - protein, 18 aa, Homo Sapiens
ATEHLSTLSEKAKPALED

SEQ ID NO: 681 - protein, 18 aa, Homo Sapiens
TEHLSTLSEKAKPALEDL

SEQ ID NO: 682 - protein, 18 aa, Homo Sapiens
EHLSTLSEKAKPALEDLR

SEQ ID NO: 683 - protein, 18 aa, Homo Sapiens
LSTLSEKAKPALEDLRQG

SEQ ID NO: 684 - protein, 18 aa, Homo Sapiens
STLSEKAKPALEDLRQGL

SEQ ID NO: 685 - protein, 18 aa, Homo Sapiens
TLSEKAKPALEDLRQGLL

SEQ ID NO: 686 - protein, 18 aa, Homo Sapiens
LSEKAKPALEDLRQGLLP

SEQ ID NO: 687 - protein, 18 aa, Homo Sapiens
SEKAKPALEDLRQGLLPV

SEQ ID NO: 688 - protein, 18 aa, Homo Sapiens
EKAKPALEDLRQGLLPVL

SEQ ID NO: 689 - protein, 18 aa, Homo Sapiens
KAKPALEDLRQGLLPVLE

SEQ ID NO: 690 - protein, 18 aa, Homo Sapiens
AKPALEDLRQGLLPVLES

SEQ ID NO: 691 - protein, 18 aa, Homo Sapiens
PALEDLRQGLLPVLESFK

SEQ ID NO: 692 - protein, 18 aa, Homo Sapiens
ALEDLRQGLLPVLESFKV

SEQ ID NO: 693 - protein, 18 aa, Homo Sapiens
LEDLRQGLLPVLESFKVS

SEQ ID NO: 694 - protein, 18 aa, Homo Sapiens
EDLRQGLLPVLESFKVSF

SEQ ID NO: 695 - protein, 18 aa, Homo Sapiens
DLRQGLLPVLESFKVSFL

SEQ ID NO: 696 - protein, 18 aa, Homo Sapiens
LRQGLLPVLESFKVSFLS

SEQ ID NO: 697 - protein, 18 aa, Homo Sapiens
RQGLLPVLESFKVSFLSA

SEQ ID NO: 698 - protein, 18 aa, Homo Sapiens
QGLLPVLESFKVSFLSAL

SEQ ID NO: 699 - protein, 18 aa, Homo Sapiens
LLPVLESFKVSFLSALEE

SEQ ID NO: 700 - protein, 18 aa, Homo Sapiens
LPVLESFKVSFLSALEEY

SEQ ID NO: 701 - protein, 18 aa, Homo Sapiens
PVLESFKVSFLSALEEYT

SEQ ID NO: 702 - protein, 18 aa, Homo Sapiens
VLESFKVSFLSALEEYTK

SEQ ID NO: 703 - protein, 18 aa, Homo Sapiens
LESFKVSFLSALEEYTKK

SEQ ID NO: 704 - protein, 18 aa, Homo Sapiens
ESFKVSFLSALEEYTKKL

SEQ ID NO: 705 - protein, 18 aa, Homo Sapiens
SFKVSFLSALEEYTKKLN

SEQ ID NO: 706 - protein, 18 aa, Homo Sapiens
FKVSFLSALEEYTKKLNT

SEQ ID NO: 707 - protein, 17 aa, Homo Sapiens
AEYHAKATEHLSTLSEK

SEQ ID NO: 708 - protein, 17 aa, Homo Sapiens
EYHAKATEHLSTLSEKA

SEQ ID NO: 709 - protein, 17 aa, Homo Sapiens
YHAKATEHLSTLSEKAK

SEQ ID NO: 710 - protein, 17 aa, Homo Sapiens
HAKATEHLSTLSEKAKP

SEQ ID NO: 711 - protein, 17 aa, Homo Sapiens
AKATEHLSTLSEKAKPA

SEQ ID NO: 712 - protein, 17 aa, Homo Sapiens
KATEHLSTLSEKAKPAL

SEQ ID NO: 713 - protein, 17 aa, Homo Sapiens
ATEHLSTLSEKAKPALE

SEQ ID NO: 714 - protein, 17 aa, Homo Sapiens
TEHLSTLSEKAKPALED

SEQ ID NO: 715 - protein, 17 aa, Homo Sapiens
EHLSTLSEKAKPALEDL

SEQ ID NO: 716 - protein, 17 aa, Homo Sapiens
HLSTLSEKAKPALEDLR

SEQ ID NO: 717 - protein, 17 aa, Homo Sapiens
LSTLSEKAKPALEDLRQ

SEQ ID NO: 718 - protein, 17 aa, Homo Sapiens
STLSEKAKPALEDLRQG

SEQ ID NO: 719 - protein, 17 aa, Homo Sapiens
TLSEKAKPALEDLRQGL

SEQ ID NO: 720 - protein, 17 aa, Homo Sapiens
LSEKAKPALEDLRQGLL

SEQ ID NO: 721 - protein, 17 aa, Homo Sapiens
SEKAKPALEDLRQGLLP

SEQ ID NO: 722 - protein, 17 aa, Homo Sapiens
EKAKPALEDLRQGLLPV

SEQ ID NO: 723 - protein, 17 aa, Homo Sapiens
KAKPALEDLRQGLLPVL

SEQ ID NO: 724 - protein, 17 aa, Homo Sapiens
AKPALEDLRQGLLPVLE

SEQ ID NO: 725 - protein, 17 aa, Homo Sapiens
KPALEDLRQGLLPVLES

SEQ ID NO: 726 - protein, 17 aa, Homo Sapiens
PALEDLRQGLLPVLESF

SEQ ID NO: 727 - protein, 17 aa, Homo Sapiens
ALEDLRQGLLPVLESFK

SEQ ID NO: 728 - protein, 17 aa, Homo Sapiens
LEDLRQGLLPVLESFKV

SEQ ID NO: 729 - protein, 17 aa, Homo Sapiens
EDLRQGLLPVLESFKVS

SEQ ID NO: 730 - protein, 17 aa, Homo Sapiens
DLRQGLLPVLESFKVSF

SEQ ID NO: 731 - protein, 17 aa, Homo Sapiens
LRQGLLPVLESFKVSFL

SEQ ID NO: 732 - protein, 17 aa, Homo Sapiens
RQGLLPVLESFKVSFLS

SEQ ID NO: 733 - protein, 17 aa, Homo Sapiens
QGLLPVLESFKVSFLSA

SEQ ID NO: 734 - protein, 17 aa, Homo Sapiens
GLLPVLESFKVSFLSAL

SEQ ID NO: 735 - protein, 17 aa, Homo Sapiens
LLPVLESFKVSFLSALE

SEQ ID NO: 736 - protein, 17 aa, Homo Sapiens
LPVLESFKVSFLSALEE

SEQ ID NO: 737 - protein, 17 aa, Homo Sapiens
PVLESFKVSFLSALEEY

SEQ ID NO: 738 - protein, 17 aa, Homo Sapiens
VLESFKVSFLSALEEYT

SEQ ID NO: 739 - protein, 17 aa, Homo Sapiens
LESFKVSFLSALEEYTK

SEQ ID NO: 740 - protein, 17 aa, Homo Sapiens
ESFKVSFLSALEEYTKK

SEQ ID NO: 741 - protein, 17 aa, Homo Sapiens
SFKVSFLSALEEYTKKL

SEQ ID NO: 742 - protein, 17 aa, Homo Sapiens
FKVSFLSALEEYTKKLN

SEQ ID NO: 743 - protein, 17 aa, Homo Sapiens
KVSFLSALEEYTKKLNT

SEQ ID NO: 744 - protein, 17 aa, Homo Sapiens
VSFLSALEEYTKKLNTQ

SEQ ID NO: 745 - protein, 16 aa, Homo Sapiens
AEYHAKATEHLSTLSE

SEQ ID NO: 746 - protein, 16 aa, Homo Sapiens
EYHAKATEHLSTLSEK

SEQ ID NO: 747 - protein, 16 aa, Homo Sapiens
YHAKATEHLSTLSEKA

SEQ ID NO: 748 - protein, 16 aa, Homo Sapiens
HAKATEHLSTLSEKAK

SEQ ID NO: 749 - protein, 16 aa, Homo Sapiens
AKATEHLSTLSEKAKP

SEQ ID NO: 750 - protein, 16 aa, Homo Sapiens
KATEHLSTLSEKAKPA

SEQ ID NO: 751 - protein, 16 aa, Homo Sapiens
ATEHLSTLSEKAKPAL

SEQ ID NO: 752 - protein, 16 aa, Homo Sapiens
TEHLSTLSEKAKPALE

SEQ ID NO: 753 - protein, 16 aa, Homo Sapiens
EHLSTLSEKAKPALED

SEQ ID NO: 754 - protein, 16 aa, Homo Sapiens
HLSTLSEKAKPALEDL

SEQ ID NO: 755 - protein, 16 aa, Homo Sapiens
LSTLSEKAKPALEDLR

SEQ ID NO: 756 - protein, 16 aa, Homo Sapiens
STLSEKAKPALEDLRQ

SEQ ID NO: 757 - protein, 16 aa, Homo Sapiens
TLSEKAKPALEDLRQG

SEQ ID NO: 758 - protein, 16 aa, Homo Sapiens
LSEKAKPALEDLRQGL

SEQ ID NO: 759 - protein, 16 aa, Homo Sapiens
SEKAKPALEDLRQGLL

SEQ ID NO: 760 - protein, 16 aa, Homo Sapiens
EKAKPALEDLRQGLLP

SEQ ID NO: 761 - protein, 16 aa, Homo Sapiens
KAKPALEDLRQGLLPV

SEQ ID NO: 762 - protein, 16 aa, Homo Sapiens
AKPALEDLRQGLLPVL

SEQ ID NO: 763 - protein, 16 aa, Homo Sapiens
KPALEDLRQGLLPVLE

SEQ ID NO: 764 - protein, 16 aa, Homo Sapiens
PALEDLRQGLLPVLES

SEQ ID NO: 765 - protein, 16 aa, Homo Sapiens
ALEDLRQGLLPVLESF

SEQ ID NO: 766 - protein, 16 aa, Homo Sapiens
LEDLRQGLLPVLESFK

SEQ ID NO: 767 - protein, 16 aa, Homo Sapiens
EDLRQGLLPVLESFKV

SEQ ID NO: 768 - protein, 16 aa, Homo Sapiens
DLRQGLLPVLESFKVS

SEQ ID NO: 769 - protein, 16 aa, Homo Sapiens
LRQGLLPVLESFKVSF

SEQ ID NO: 770 - protein, 16 aa, Homo Sapiens
RQGLLPVLESFKVSFL

SEQ ID NO: 771 - protein, 16 aa, Homo Sapiens
QGLLPVLESFKVSFLS

SEQ ID NO: 772 - protein, 16 aa, Homo Sapiens
GLLPVLESFKVSFLSA

SEQ ID NO: 773 - protein, 16 aa, Homo Sapiens
LLPVLESFKVSFLSAL

SEQ ID NO: 774 - protein, 16 aa, Homo Sapiens
LPVLESFKVSFLSALE

SEQ ID NO: 775 - protein, 16 aa, Homo Sapiens
PVLESFKVSFLSALEE

SEQ ID NO: 776 - protein, 16 aa, Homo Sapiens
VLESFKVSFLSALEEY

SEQ ID NO: 777 - protein, 16 aa, Homo Sapiens
LESFKVSFLSALEEYT

SEQ ID NO: 778 - protein, 16 aa, Homo Sapiens
ESFKVSFLSALEEYTK

SEQ ID NO: 779 - protein, 16 aa, Homo Sapiens
SFKVSFLSALEEYTKK

SEQ ID NO: 780 - protein, 16 aa, Homo Sapiens
FKVSFLSALEEYTKKL

SEQ ID NO: 781 - protein, 16 aa, Homo Sapiens
KVSFLSALEEYTKKLN

SEQ ID NO: 782 - protein, 16 aa, Homo Sapiens
VSFLSALEEYTKKLNT

SEQ ID NO: 783 - protein, 16 aa, Homo Sapiens
SFLSALEEYTKKLNTQ

SEQ ID NO: 784 - protein, 15 aa, Homo Sapiens
AEYHAKATEHLSTLS

SEQ ID NO: 785 - protein, 15 aa, Homo Sapiens
EYHAKATEHLSTLSE

SEQ ID NO: 786 - protein, 15 aa, Homo Sapiens
YHAKATEHLSTLSEK

SEQ ID NO: 787 - protein, 15 aa, Homo Sapiens
HAKATEHLSTLSEKA

SEQ ID NO: 788 - protein, 15 aa, Homo Sapiens
AKATEHLSTLSEKAK

SEQ ID NO: 789 - protein, 15 aa, Homo Sapiens
KATEHLSTLSEKAKP

SEQ ID NO: 790 - protein, 15 aa, Homo Sapiens
ATEHLSTLSEKAKPA

SEQ ID NO: 791 - protein, 15 aa, Homo Sapiens
TEHLSTLSEKAKPAL

SEQ ID NO: 792 - protein, 15 aa, Homo Sapiens
EHLSTLSEKAKPALE

SEQ ID NO: 793 - protein, 15 aa, Homo Sapiens
HLSTLSEKAKPALED

SEQ ID NO: 794 - protein, 15 aa, Homo Sapiens
LSTLSEKAKPALEDL

SEQ ID NO: 795 - protein, 15 aa, Homo Sapiens
STLSEKAKPALEDLR

SEQ ID NO: 796 - protein, 15 aa, Homo Sapiens
TLSEKAKPALEDLRQ

SEQ ID NO: 797 - protein, 15 aa, Homo Sapiens
LSEKAKPALEDLRQG

SEQ ID NO: 798 - protein, 15 aa, Homo Sapiens
SEKAKPALEDLRQGL

SEQ ID NO: 799 - protein, 15 aa, Homo Sapiens
EKAKPALEDLRQGLL

SEQ ID NO: 800 - protein, 15 aa, Homo Sapiens
KAKPALEDLRQGLLP

SEQ ID NO: 801 - protein, 15 aa, Homo Sapiens
AKPALEDLRQGLLPV

SEQ ID NO: 802 - protein, 15 aa, Homo Sapiens
KPALEDLRQGLLPVL

SEQ ID NO: 803 - protein, 15 aa, Homo Sapiens
PALEDLRQGLLPVLE

SEQ ID NO: 804 - protein, 15 aa, Homo Sapiens
ALEDLRQGLLPVLES

SEQ ID NO: 805 - protein, 15 aa, Homo Sapiens
LEDLRQGLLPVLESF

SEQ ID NO: 806 - protein, 15 aa, Homo Sapiens
EDLRQGLLPVLESFK

SEQ ID NO: 807 - protein, 15 aa, Homo Sapiens
DLRQGLLPVLESFKV

SEQ ID NO: 808 - protein, 15 aa, Homo Sapiens
LRQGLLPVLESFKVS

SEQ ID NO: 809 - protein, 15 aa, Homo Sapiens
RQGLLPVLESFKVSF

SEQ ID NO: 810 - protein, 15 aa, Homo Sapiens
QGLLPVLESFKVSFL

SEQ ID NO: 811 - protein, 15 aa, Homo Sapiens
GLLPVLESFKVSFLS

SEQ ID NO: 812 - protein, 15 aa, Homo Sapiens
LLPVLESFKVSFLSA

SEQ ID NO: 813 - protein, 15 aa, Homo Sapiens
LPVLESFKVSFLSAL

SEQ ID NO: 814 - protein, 15 aa, Homo Sapiens
PVLESFKVSFLSALE

SEQ ID NO: 815 - protein, 15 aa, Homo Sapiens
VLESFKVSFLSALEE

SEQ ID NO: 816 - protein, 15 aa, Homo Sapiens
LESFKVSFLSALEEY

SEQ ID NO: 817 - protein, 15 aa, Homo Sapiens
ESFKVSFLSALEEYT

SEQ ID NO: 818 - protein, 15 aa, Homo Sapiens
SFKVSFLSALEEYTK

SEQ ID NO: 819 - protein, 15 aa, Homo Sapiens
FKVSFLSALEEYTKK

SEQ ID NO: 820 - protein, 15 aa, Homo Sapiens
KVSFLSALEEYTKKL

SEQ ID NO: 821 - protein, 15 aa, Homo Sapiens
VSFLSALEEYTKKLN

SEQ ID NO: 822 - protein, 15 aa, Homo Sapiens
SFLSALEEYTKKLNT

SEQ ID NO: 823 - protein, 15 aa, Homo Sapiens
FLSALEEYTKKLNTQ

SEQ ID NO: 824 - protein, 14 aa, Homo Sapiens
AEYHAKATEHLSTL

SEQ ID NO: 825 - protein, 14 aa, Homo Sapiens
EYHAKATEHLSTLS

SEQ ID NO: 826 - protein, 14 aa, Homo Sapiens
YHAKATEHLSTLSE

SEQ ID NO: 827 - protein, 14 aa, Homo Sapiens
HAKATEHLSTLSEK

SEQ ID NO: 828 - protein, 14 aa, Homo Sapiens
AKATEHLSTLSEKA

SEQ ID NO: 829 - protein, 14 aa, Homo Sapiens
KATEHLSTLSEKAK

SEQ ID NO: 830 - protein, 14 aa, Homo Sapiens
ATEHLSTLSEKAKP

SEQ ID NO: 831 - protein, 14 aa, Homo Sapiens
TEHLSTLSEKAKPA

SEQ ID NO: 832 - protein, 14 aa, Homo Sapiens
EHLSTLSEKAKPAL

SEQ ID NO: 833 - protein, 14 aa, Homo Sapiens
HLSTLSEKAKPALE

SEQ ID NO: 834 - protein, 14 aa, Homo Sapiens
LSTLSEKAKPALED

SEQ ID NO: 835 - protein, 14 aa, Homo Sapiens
STLSEKAKPALEDL

SEQ ID NO: 836 - protein, 14 aa, Homo Sapiens
TLSEKAKPALEDLR

SEQ ID NO: 837 - protein, 14 aa, Homo Sapiens
LSEKAKPALEDLRQ

SEQ ID NO: 838 - protein, 14 aa, Homo Sapiens
SEKAKPALEDLRQG

SEQ ID NO: 839 - protein, 14 aa, Homo Sapiens
EKAKPALEDLRQGL

SEQ ID NO: 840 - protein, 14 aa, Homo Sapiens
KAKPALEDLRQGLL

SEQ ID NO: 841 - protein, 14 aa, Homo Sapiens
AKPALEDLRQGLLP

SEQ ID NO: 842 - protein, 14 aa, Homo Sapiens
KPALEDLRQGLLPV

SEQ ID NO: 843 - protein, 14 aa, Homo Sapiens
PALEDLRQGLLPVL

SEQ ID NO: 844 - protein, 14 aa, Homo Sapiens
ALEDLRQGLLPVLE

SEQ ID NO: 845 - protein, 14 aa, Homo Sapiens
LEDLRQGLLPVLES

SEQ ID NO: 846 - protein, 14 aa, Homo Sapiens
EDLRQGLLPVLESF

SEQ ID NO: 847 - protein, 14 aa, Homo Sapiens
DLRQGLLPVLESFK

SEQ ID NO: 848 - protein, 14 aa, Homo Sapiens
LRQGLLPVLESFKV

SEQ ID NO: 849 - protein, 14 aa, Homo Sapiens
RQGLLPVLESFKVS

SEQ ID NO: 850 - protein, 14 aa, Homo Sapiens
QGLLPVLESFKVSF

SEQ ID NO: 851 - protein, 14 aa, Homo Sapiens
GLLPVLESFKVSFL

SEQ ID NO: 852 - protein, 14 aa, Homo Sapiens
LLPVLESFKVSFLS

SEQ ID NO: 853 - protein, 14 aa, Homo Sapiens
LPVLESFKVSFLSA

SEQ ID NO: 854 - protein, 14 aa, Homo Sapiens
PVLESFKVSFLSAL

SEQ ID NO: 855 - protein, 14 aa, Homo Sapiens
VLESFKVSFLSALE

SEQ ID NO: 856 - protein, 14 aa, Homo Sapiens
LESFKVSFLSALEE

SEQ ID NO: 857 - protein, 14 aa, Homo Sapiens
ESFKVSFLSALEEY

SEQ ID NO: 858 - protein, 14 aa, Homo Sapiens
SFKVSFLSALEEYT

SEQ ID NO: 859 - protein, 14 aa, Homo Sapiens
FKVSFLSALEEYTK

SEQ ID NO: 860 - protein, 14 aa, Homo Sapiens
KVSFLSALEEYTKK

SEQ ID NO: 861 - protein, 14 aa, Homo Sapiens
VSFLSALEEYTKKL

SEQ ID NO: 862 - protein, 14 aa, Homo Sapiens
SFLSALEEYTKKLN

SEQ ID NO: 863 - protein, 14 aa, Homo Sapiens
FLSALEEYTKKLNT

SEQ ID NO: 864 - protein, 14 aa, Homo Sapiens
LSALEEYTKKLNTQ

SEQ ID NO: 865 - protein, 13 aa, Homo Sapiens
AEYHAKATEHLST

SEQ ID NO: 866 - protein, 13 aa, Homo Sapiens
EYHAKATEHLSTL

SEQ ID NO: 867 - protein, 13 aa, Homo Sapiens
YHAKATEHLSTLS

SEQ ID NO: 868 - protein, 13 aa, Homo Sapiens
HAKATEHLSTLSE

SEQ ID NO: 869 - protein, 13 aa, Homo Sapiens
AKATEHLSTLSEK

SEQ ID NO: 870 - protein, 13 aa, Homo Sapiens
KATEHLSTLSEKA

SEQ ID NO: 871 - protein, 13 aa, Homo Sapiens
ATEHLSTLSEKAK

SEQ ID NO: 872 - protein, 13 aa, Homo Sapiens
TEHLSTLSEKAKP

SEQ ID NO: 873 - protein, 13 aa, Homo Sapiens
EHLSTLSEKAKPA

SEQ ID NO: 874 - protein, 13 aa, Homo Sapiens
HLSTLSEKAKPAL

SEQ ID NO: 875 - protein, 13 aa, Homo Sapiens
LSTLSEKAKPALE

SEQ ID NO: 876 - protein, 13 aa, Homo Sapiens
STLSEKAKPALED

SEQ ID NO: 877 - protein, 13 aa, Homo Sapiens
TLSEKAKPALEDL

SEQ ID NO: 878 - protein, 13 aa, Homo Sapiens
LSEKAKPALEDLR

SEQ ID NO: 879 - protein, 13 aa, Homo Sapiens
SEKAKPALEDLRQ

SEQ ID NO: 880 - protein, 13 aa, Homo Sapiens
EKAKPALEDLRQG

SEQ ID NO: 881 - protein, 13 aa, Homo Sapiens
KAKPALEDLRQGL

SEQ ID NO: 882 - protein, 13 aa, Homo Sapiens
AKPALEDLRQGLL

SEQ ID NO: 883 - protein, 13 aa, Homo Sapiens
KPALEDLRQGLLP

SEQ ID NO: 884 - protein, 13 aa, Homo Sapiens
PALEDLRQGLLPV

SEQ ID NO: 885 - protein, 13 aa, Homo Sapiens
ALEDLRQGLLPVL

SEQ ID NO: 886 - protein, 13 aa, Homo Sapiens
LEDLRQGLLPVLE

SEQ ID NO: 887 - protein, 13 aa, Homo Sapiens
EDLRQGLLPVLES

SEQ ID NO: 888 - protein, 13 aa, Homo Sapiens
DLRQGLLPVLESF

SEQ ID NO: 889 - protein, 13 aa, Homo Sapiens
LRQGLLPVLESFK

SEQ ID NO: 890 - protein, 13 aa, Homo Sapiens
RQGLLPVLESFKV

SEQ ID NO: 891 - protein, 13 aa, Homo Sapiens
QGLLPVLESFKVS

SEQ ID NO: 892 - protein, 13 aa, Homo Sapiens
GLLPVLESFKVSF

SEQ ID NO: 893 - protein, 13 aa, Homo Sapiens
LLPVLESFKVSFL

SEQ ID NO: 894 - protein, 13 aa, Homo Sapiens
LPVLESFKVSFLS

SEQ ID NO: 895 - protein, 13 aa, Homo Sapiens
PVLESFKVSFLSA

SEQ ID NO: 896 - protein, 13 aa, Homo Sapiens
VLESFKVSFLSAL

SEQ ID NO: 897 - protein, 13 aa, Homo Sapiens
LESFKVSFLSALE

SEQ ID NO: 898 - protein, 13 aa, Homo Sapiens
ESFKVSFLSALEE

SEQ ID NO: 899 - protein, 13 aa, Homo Sapiens
SFKVSFLSALEEY

SEQ ID NO: 900 - protein, 13 aa, Homo Sapiens
FKVSFLSALEEYT

SEQ ID NO: 901 - protein, 13 aa, Homo Sapiens
KVSFLSALEEYTK

SEQ ID NO: 902 - protein, 13 aa, Homo Sapiens
VSFLSALEEYTKK

SEQ ID NO: 903 - protein, 13 aa, Homo Sapiens
SFLSALEEYTKKL

SEQ ID NO: 904 - protein, 13 aa, Homo Sapiens
FLSALEEYTKKLN

SEQ ID NO: 905 - protein, 13 aa, Homo Sapiens
LSALEEYTKKLNT

SEQ ID NO: 906 - protein, 13 aa, Homo Sapiens
SALEEYTKKLNTQ

SEQ ID NO: 907 - protein, 12 aa, Homo Sapiens
AEYHAKATEHLS

SEQ ID NO: 908 - protein, 12 aa, Homo Sapiens
EYHAKATEHLST

SEQ ID NO: 909 - protein, 12 aa, Homo Sapiens
YHAKATEHLSTL

SEQ ID NO: 910 - protein, 12 aa, Homo Sapiens
HAKATEHLSTLS

SEQ ID NO: 911 - protein, 12 aa, Homo Sapiens
AKATEHLSTLSE

SEQ ID NO: 912 - protein, 12 aa, Homo Sapiens
KATEHLSTLSEK

SEQ ID NO: 913 - protein, 12 aa, Homo Sapiens
ATEHLSTLSEKA

SEQ ID NO: 914 - protein, 12 aa, Homo Sapiens
TEHLSTLSEKAK

SEQ ID NO: 915 - protein, 12 aa, Homo Sapiens
EHLSTLSEKAKP

SEQ ID NO: 916 - protein, 12 aa, Homo Sapiens
HLSTLSEKAKPA

SEQ ID NO: 917 - protein, 12 aa, Homo Sapiens
LSTLSEKAKPAL

SEQ ID NO: 918 - protein, 12 aa, Homo Sapiens
STLSEKAKPALE

SEQ ID NO: 919 - protein, 12 aa, Homo Sapiens
TLSEKAKPALED

SEQ ID NO: 920 - protein, 12 aa, Homo Sapiens
LSEKAKPALEDL

SEQ ID NO: 921 - protein, 12 aa, Homo Sapiens
SEKAKPALEDLR

SEQ ID NO: 922 - protein, 12 aa, Homo Sapiens
EKAKPALEDLRQ

SEQ ID NO: 923 - protein, 12 aa, Homo Sapiens
KAKPALEDLRQG

SEQ ID NO: 924 - protein, 12 aa, Homo Sapiens
AKPALEDLRQGL

SEQ ID NO: 925 - protein, 12 aa, Homo Sapiens
KPALEDLRQGLL

SEQ ID NO: 926 - protein, 12 aa, Homo Sapiens
PALEDLRQGLLP

SEQ ID NO: 927 - protein, 12 aa, Homo Sapiens
ALEDLRQGLLPV

SEQ ID NO: 928 - protein, 12 aa, Homo Sapiens
LEDLRQGLLPVL

SEQ ID NO: 929 - protein, 12 aa, Homo Sapiens
EDLRQGLLPVLE

SEQ ID NO: 930 - protein, 12 aa, Homo Sapiens
DLRQGLLPVLES

SEQ ID NO: 931 - protein, 12 aa, Homo Sapiens
LRQGLLPVLESF

SEQ ID NO: 932 - protein, 12 aa, Homo Sapiens
RQGLLPVLESFK

SEQ ID NO: 933 - protein, 12 aa, Homo Sapiens
QGLLPVLESFKV

SEQ ID NO: 934 - protein, 12 aa, Homo Sapiens
GLLPVLESFKVS

SEQ ID NO: 935 - protein, 12 aa, Homo Sapiens
LLPVLESFKVSF

SEQ ID NO: 936 - protein, 12 aa, Homo Sapiens
LPVLESFKVSFL

SEQ ID NO: 937 - protein, 12 aa, Homo Sapiens
PVLESFKVSFLS

SEQ ID NO: 938 - protein, 12 aa, Homo Sapiens
VLESFKVSFLSA

SEQ ID NO: 939 - protein, 12 aa, Homo Sapiens
LESFKVSFLSAL

SEQ ID NO: 940 - protein, 12 aa, Homo Sapiens
ESFKVSFLSALE

SEQ ID NO: 941 - protein, 12 aa, Homo Sapiens
SFKVSFLSALEE

SEQ ID NO: 942 - protein, 12 aa, Homo Sapiens
FKVSFLSALEEY

SEQ ID NO: 943 - protein, 12 aa, Homo Sapiens
KVSFLSALEEYT

SEQ ID NO: 944 - protein, 12 aa, Homo Sapiens
VSFLSALEEYTK

SEQ ID NO: 945 - protein, 12 aa, Homo Sapiens
SFLSALEEYTKK

SEQ ID NO: 946 - protein, 12 aa, Homo Sapiens
FLSALEEYTKKL

SEQ ID NO: 947 - protein, 12 aa, Homo Sapiens
LSALEEYTKKLN

SEQ ID NO: 948 - protein, 12 aa, Homo Sapiens
SALEEYTKKLNT

SEQ ID NO: 949 - protein, 12 aa, Homo Sapiens
ALEEYTKKLNTQ

SEQ ID NO: 950 - protein, 11 aa, Homo Sapiens
AEYHAKATEHL

SEQ ID NO: 951 - protein, 11 aa, Homo Sapiens
EYHAKATEHLS

SEQ ID NO: 952 - protein, 11 aa, Homo Sapiens
YHAKATEHLST

SEQ ID NO: 953 - protein, 11 aa, Homo Sapiens
HAKATEHLSTL

SEQ ID NO: 954 - protein, 11 aa, Homo Sapiens
AKATEHLSTLS

SEQ ID NO: 955 - protein, 11 aa, Homo Sapiens
KATEHLSTLSE

SEQ ID NO: 956 - protein, 11 aa, Homo Sapiens
ATEHLSTLSEK

SEQ ID NO: 957 - protein, 11 aa, Homo Sapiens
TEHLSTLSEKA

SEQ ID NO: 958 - protein, 11 aa, Homo Sapiens
EHLSTLSEKAK

SEQ ID NO: 959 - protein, 11 aa, Homo Sapiens
HLSTLSEKAKP

SEQ ID NO: 960 - protein, 11 aa, Homo Sapiens
LSTLSEKAKPA

SEQ ID NO: 961 - protein, 11 aa, Homo Sapiens
STLSEKAKPAL

SEQ ID NO: 962 - protein, 11 aa, Homo Sapiens
TLSEKAKPALE

SEQ ID NO: 963 - protein, 11 aa, Homo Sapiens
LSEKAKPALED

SEQ ID NO: 964 - protein, 11 aa, Homo Sapiens
SEKAKPALEDL

SEQ ID NO: 965 - protein, 11 aa, Homo Sapiens
EKAKPALEDLR

SEQ ID NO: 966 - protein, 11 aa, Homo Sapiens
KAKPALEDLRQ

SEQ ID NO: 967 - protein, 11 aa, Homo Sapiens
AKPALEDLRQG

SEQ ID NO: 968 - protein, 11 aa, Homo Sapiens
KPALEDLRQGL

SEQ ID NO: 969 - protein, 11 aa, Homo Sapiens
PALEDLRQGLL

SEQ ID NO: 970 - protein, 11 aa, Homo Sapiens
ALEDLRQGLLP

SEQ ID NO: 971 - protein, 11 aa, Homo Sapiens
LEDLRQGLLPV

SEQ ID NO: 972 - protein, 11 aa, Homo Sapiens
EDLRQGLLPVL

SEQ ID NO: 973 - protein, 11 aa, Homo Sapiens
DLRQGLLPVLE

SEQ ID NO: 974 - protein, 11 aa, Homo Sapiens
LRQGLLPVLES

SEQ ID NO: 975 - protein, 11 aa, Homo Sapiens
RQGLLPVLESF

SEQ ID NO: 976 - protein, 11 aa, Homo Sapiens
QGLLPVLESFK

SEQ ID NO: 977 - protein, 11 aa, Homo Sapiens
GLLPVLESFKV

SEQ ID NO: 978 - protein, 11 aa, Homo Sapiens
LLPVLESFKVS

SEQ ID NO: 979 - protein, 11 aa, Homo Sapiens
LPVLESFKVSF

SEQ ID NO: 980 - protein, 11 aa, Homo Sapiens
PVLESFKVSFL

SEQ ID NO: 981 - protein, 11 aa, Homo Sapiens
VLESFKVSFLS

SEQ ID NO: 982 - protein, 11 aa, Homo Sapiens
LESFKVSFLSA

SEQ ID NO: 983 - protein, 11 aa, Homo Sapiens
ESFKVSFLSAL

SEQ ID NO: 984 - protein, 11 aa, Homo Sapiens
SFKVSFLSALE

SEQ ID NO: 985 - protein, 11 aa, Homo Sapiens
FKVSFLSALEE

SEQ ID NO: 986 - protein, 11 aa, Homo Sapiens
KVSFLSALEEY

SEQ ID NO: 987 - protein, 11 aa, Homo Sapiens
VSFLSALEEYT

SEQ ID NO: 988 - protein, 11 aa, Homo Sapiens
SFLSALEEYTK

SEQ ID NO: 989 - protein, 11 aa, Homo Sapiens
FLSALEEYTKK

SEQ ID NO: 990 - protein, 11 aa, Homo Sapiens
LSALEEYTKKL

SEQ ID NO: 991 - protein, 11 aa, Homo Sapiens
SALEEYTKKLN

SEQ ID NO: 992 - protein, 11 aa, Homo Sapiens
ALEEYTKKLNT

SEQ ID NO: 993 - protein, 11 aa, Homo Sapiens
LEEYTKKLNTQ

SEQ ID NO: 994 - protein, 10 aa, Homo Sapiens
AEYHAKATEH

SEQ ID NO: 995 - protein, 10 aa, Homo Sapiens
EYHAKATEHL

SEQ ID NO: 996 - protein, 10 aa, Homo Sapiens
YHAKATEHLS

SEQ ID NO: 997 - protein, 10 aa, Homo Sapiens
HAKATEHLST

SEQ ID NO: 998 - protein, 10 aa, Homo Sapiens
AKATEHLSTL

SEQ ID NO: 999 - protein, 10 aa, Homo Sapiens
KATEHLSTLS

SEQ ID NO: 1000 - protein, 10 aa, Homo Sapiens
ATEHLSTLSE

SEQ ID NO: 1001 - protein, 10 aa, Homo Sapiens
TEHLSTLSEK

SEQ ID NO: 1002 - protein, 10 aa, Homo Sapiens
EHLSTLSEKA

SEQ ID NO: 1003 - protein, 10 aa, Homo Sapiens
HLSTLSEKAK

SEQ ID NO: 1004 - protein, 10 aa, Homo Sapiens
LSTLSEKAKP

SEQ ID NO: 1005 - protein, 10 aa, Homo Sapiens
STLSEKAKPA

SEQ ID NO: 1006 - protein, 10 aa, Homo Sapiens
TLSEKAKPAL

SEQ ID NO: 1007 - protein, 10 aa, Homo Sapiens
LSEKAKPALE

SEQ ID NO: 1008 - protein, 10 aa, Homo Sapiens
SEKAKPALED

SEQ ID NO: 1009 - protein, 10 aa, Homo Sapiens
EKAKPALEDL

SEQ ID NO: 1010 - protein, 10 aa, Homo Sapiens
KAKPALEDLR

SEQ ID NO: 1011 - protein, 10 aa, Homo Sapiens
AKPALEDLRQ

SEQ ID NO: 1012 - protein, 10 aa, Homo Sapiens
KPALEDLRQG

SEQ ID NO: 1013 - protein, 10 aa, Homo Sapiens
PALEDLRQGL

SEQ ID NO: 1014 - protein, 10 aa, Homo Sapiens
ALEDLRQGLL

SEQ ID NO: 1015 - protein, 10 aa, Homo Sapiens
LEDLRQGLLP

SEQ ID NO: 1016 - protein, 10 aa, Homo Sapiens
EDLRQGLLPV

SEQ ID NO: 1017 - protein, 10 aa, Homo Sapiens
DLRQGLLPVL

SEQ ID NO: 1018 - protein, 10 aa, Homo Sapiens
LRQGLLPVLE

SEQ ID NO: 1019 - protein, 10 aa, Homo Sapiens
RQGLLPVLES

SEQ ID NO: 1020 - protein, 10 aa, Homo Sapiens
QGLLPVLESF

SEQ ID NO: 1021 - protein, 10 aa, Homo Sapiens
GLLPVLESFK

SEQ ID NO: 1022 - protein, 10 aa, Homo Sapiens
LLPVLESFKV

SEQ ID NO: 1023 - protein, 10 aa, Homo Sapiens
LPVLESFKVS

SEQ ID NO: 1024 - protein, 10 aa, Homo Sapiens
PVLESFKVSF

SEQ ID NO: 1025 - protein, 10 aa, Homo Sapiens
VLESFKVSFL

SEQ ID NO: 1026 - protein, 10 aa, Homo Sapiens
LESFKVSFLS

SEQ ID NO: 1027 - protein, 10 aa, Homo Sapiens
ESFKVSFLSA

SEQ ID NO: 1028 - protein, 10 aa, Homo Sapiens
SFKVSFLSAL

SEQ ID NO: 1029 - protein, 10 aa, Homo Sapiens
FKVSFLSALE

SEQ ID NO: 1030 - protein, 10 aa, Homo Sapiens
KVSFLSALEE

SEQ ID NO: 1031 - protein, 10 aa, Homo Sapiens
VSFLSALEEY

SEQ ID NO: 1032 - protein, 10 aa, Homo Sapiens
SFLSALEEYT

SEQ ID NO: 1033 - protein, 10 aa, Homo Sapiens
FLSALEEYTK

SEQ ID NO: 1034 - protein, 10 aa, Homo Sapiens
LSALEEYTKK

SEQ ID NO: 1035 - protein, 10 aa, Homo Sapiens
SALEEYTKKL